(19) United States
(12) Patent Application Publication
(10) Pub. No.: US 2025/0242052 A1
(43) Pub. Date: Jul. 31, 2025

(54) DNA THERAPEUTIC ENCODING AN ANTIBODY OR ANTIGEN BINDING FRAGMENT

(71) Applicants: Hong Jiang, Vega Alta, PR (US); AEGIS Life, Inc., San Diego, CA (US)

(72) Inventors: Hong Jiang, Vega Alta (PR); Alexander Sevy, San Diego, CA (US); Brian Abel, San Diego, CA (US); Arun Raturi, San Diego, CA (US); Thornton Thompson, San Diego, CA (US)

(21) Appl. No.: 18/838,923

(22) PCT Filed: Apr. 18, 2023
(86) PCT No.: PCT/US2023/019003

§ 371 Date: Aug. 15, 2024

Related U.S. Application Data
(60) Provisional application No. 63/332,386, filed on Apr. 19, 2022.
(60) Provisional application No. 63/347,120, filed on May 31, 2022.
(60) Provisional application No. 63/357,953, filed on Jul. 1, 2022.

(51) Int. Cl. A61K 48/00; A61K 39/00; C07K 16/10

(52) U.S. Cl. CPC A61K 48/005; A61K 48/0083; C07K 16/1003; A61K 2039/505; C07K 2317/524; C07K 2317/526; C07K 2317/53; C07K 2317/569

(57) ABSTRACT

The disclosure includes systems comprising non-viral DNA vectors encapsulated in lipid vesicles that are designed to achieve sustained therapeutically relevant expression levels in blood plasma of therapeutic proteins, polypeptides, or peptides. The system can express antibodies or antigen-binding fragments at sustained and therapeutically relevant levels. The system can be administered to a subject via local or systemic routes such as intravenous (IV) or intramuscular (IM), and repeat dosing produces an additive effect. For local administration, localized expression can be achieved. Applications span treatments for various conditions, including infectious diseases, cancers, and inflammatory diseases.

### RELATED APPLICATIONS

This patent application is a U.S. National Stage patent Application under 35 U.S.C. 371 of International Application No. PCT/US2023/019003, filed Apr. 18, 2023, which claims priority to U.S. Provisional Patent Applications 63/332,386, filed on Apr. 19, 2022, 63/347,120, filed on May 31, 2022 and 63/357,953 filed on Jul. 1, 2022, which are incorporated herein by reference in their entirety as part of the present disclosure.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

The instant application contains a Sequence Listing which has been submitted electronically in XML format and is hereby incorporated by reference in its entirety. Said XML copy, created on Dec. 12, 2024, is named 66023.2US01 Sequence Listing.xml and is 123 kilobytes in size.

### BACKGROUND

Infusion of antibody therapeutics is an established method for treating acute life-threatening infectious disease and for protecting individuals that are immunocompromised or as therapies for other illnesses. Many therapeutic antibodies require careful storage to preserve their therapeutic activity. Further, infused antibody concentrations may fall over time due to serum protein turnover or due to anti-drug antibody responses which neutralize the infused antibody. There exists a need for improved modalities to deliver clinically relevant and durable levels of antibodies to a subject.

### BRIEF SUMMARY

Described herein is a platform and associated methods for producing desired antibodies or antigen binding fragments in a subject. In some instances, the antibodies or antigen binding fragments are expressed by the subject after transfection of subject tissue (e.g., muscle tissue) with stable, recombinant DNA (e.g., a plasmid or multiple plasmids) encoding the desired antibody or antigen binding fragment thereof. In some instances, the recombinant DNA is transfected using a formulation which allows high efficiency transfection of subject tissue. In some instances, the formulation comprises lipid vesicles which envelop the DNA and contain a small fusogenic protein that leads to highly efficient transfection of target cells in the tissue of the subject. The encoded antibodies are then expressed and secreted by the subject's own cells at a level sufficient to be clinically relevant (e.g., having therapeutic or prophylactic activity). Surprisingly, this platform is sufficiently adaptable such that a wide variety of antibodies and different antibody formats (e.g., VHH formats, etc.) can be encoded into a DNA vector (e.g., a plasmid) and introduced into the subject to produce clinically relevant antibody or antigen binding fragment titer in the subject without the need for substantial vector optimization. The systems and methods provided herein have advantages over administration of exogenous antibodies to a subject because there is no need for the development of extensive protein expression, purification, and quality control protocols required for protein antibodies. Furthermore, the cost of administering antibodies using this novel approach is expected to be far lower than conventional administration of infused antibodies. The flexibility of the systems and methods provided herein thus present a promising platform which can be used to rapidly and readily develop antibody therapies for a wide variety of indications.

In one aspect, provided herein, is a system for expressing an antibody or an antigen binding fragment thereof in a subject, comprising: a plasmid comprising a polynucleotide sequence encoding a heavy chain variable domain of the antibody or an antigen binding fragment thereof; and wherein the plasmid is encapsulated in a lipid vesicle.

In one aspect, provided herein, is a system for expressing an antibody or an antigen binding fragment thereof in a subject, comprising: a plasmid comprising a polynucleotide sequence encoding a heavy chain variable domain of the antibody or an antigen binding fragment thereof; wherein the plasmid is encapsulated in a lipid vesicle; and wherein when the plasmid encapsulated in the lipid vesicle is administered, the subject produces a peak blood plasma level of the antibody or antigen binding fragment thereof of at least 50 ng/ml.

In some embodiments, the antibody or antigen binding fragment thereof is a single-domain antibody. In some embodiments, the antibody or antigen binding fragment thereof is a VHH antibody. In some embodiments, the heavy chain variable domain is fused to an Fc domain, optionally through a peptide linker.

In some embodiments, the plasmid encodes a full length heavy chain of the antibody. In some embodiments, the plasmid further comprises a polynucleotide sequence encoding a light chain or an antigen binding fragment of the antibody. In some embodiments, the plasmid encodes a full length light chain of the antibody. In some embodiments, the polynucleotide sequence encoding the heavy chain variable domain and the polynucleotide sequence encoding the light chain are operably coupled such that the sequences are transcribed as a single transcript. In some embodiments, the polynucleotide sequence encoding the heavy chain and the polynucleotides sequence encoding the light chain are separated by a self-cleavage peptide encoding sequence.

In some embodiments, the system comprises a second plasmid comprising a second polynucleotide sequence encoding a light chain of the antibody. In some embodiments, the light chain of the antibody is a kappa chain or a lambda chain. In some embodiments, the second plasmid is also encapsulated in a lipid vesicle.

In some embodiments, the lipid vesicle comprises a fusion-associated small transmembrane (FAST) protein. In some embodiments, the FAST protein comprises domains from one or more FAST proteins selected from p10, p14, p15, and p22. In some embodiments, the FAST protein comprises an amino acid sequence having at least 80% sequence identity to the sequence:

(SEQ ID NO: 203)
MGSGPSNFVNHAPGEAIVTGLEKGADKVAGTISHTIFVEIVSSSTGIII
AVGIFAFIFSFLYKLLQWYNRKSKNKKRKEQIREQIELGLLSYGAGVAS
LPLLNVIAHNPGSVISATPIYKGPCTGVPNSRLLQITSGTAEENTRILN
HDGRNPDGSINV.

In some embodiments, the vector comprises a promoter operably linked to the polynucleotide sequence selected from CAG, CMV, EFIA, CBh, CBA, and SFFV. In some embodiments, the plasmid comprises the CAG promoter. In some embodiments, the plasmid is a DNA plasmid.

In some embodiments, the antibody or antigen binding fragment thereof comprises an IgG1, IgG2a, IgG2b, IgG3, IgG4, IgD, IgM, IgA1, IgA2 or IgE heavy chain. In some embodiments, the antibody or antigen binding fragment thereof comprises an IgG1, IgG2a, IgG2b, IgG3, or IgG4 heavy chain. In some embodiments, the antibody comprises an IgG1 heavy chain. In some embodiments, the heavy chain variable domain comprises a sequence that is at least 80% sequence identity to AQVQLVETGGGLVQPGGSLRLSCAASXXXXXXXXXMNWVRQAPGKGPEWVSXXX XXXXXXXYTDSVKGRFTISRDNAKNTLYLQMNNLKPEDTALYYCXXXXXXX XRGQGTQVTVSS (SEQ ID NO: 101), wherein each X is independently absent or any amino acid.

In some embodiments, the antibody or antigen binding fragment comprises an Fc domain having one or more mutations or combinations of mutations selected from Arg435His (His435), Asn434Ala (A), Met428Leu/Asn434Ser (LS), Thr252Leu/Thr253Ser/Thr254Phe (LSF), Glu294delta/Thr307Pro/Asn434Tyr (C6A-66), Thr256Asn/Ala378Val/Ser383 Asn/Asn434Tyr (C6A-78), and Glu294delta (Del), wherein residue position number is based on EU numbering convention. In some embodiments, the antibody or antigen binding fragment thereof comprises an Fc domain having one or more mutations selected from M252Y, S254T, T256E, and any combination thereof, wherein residue position numbering is based on EU numbering convention.

In some embodiments, the antibody or antigen binding fragment thereof binds specifically to a viral protein. In some embodiments, the viral protein from a virus selected from a group consisting of a parvovirus, a picornavirus, a rhabdovirus, a paramyxovirus, an orthomyxovirus, a bunyavirus, a calicivirus, an arenavirus, a polyomavirus, a reovirus, a togavirus, a bunyavirus, a herpes simplex virus, a poxvirus, an adenovirus, a coxsackievirus, a flavivirus, a coronavirus, an astrovirus, an enterovirus, a rotavirus, a norovirus, a retrovirus, a papilloma virus, a parvovirus, an influenza virus, a hemorrhagic fever virus, and a rhinovirus. In some embodiments, the viral protein is from a virus select from a group consisting of Hantavirus, Rabies, Nipah, Hendra, Rift Valley Fever, Lassa, Marburg, Crimean Congo Fever, hMPV, RSV, Hepatitis A, Hepatitis B, Hepatitis C, Hepatitis D, Hepatitis E, Norovirus, Monkeypox, Coxpox, Japanese Encephalitis, Yellow Fever, HSV-1, HSV-2, MERS, ChickenPox, Hand, Foot and Mouth, CMV (HHV-5), Equine Encephalitis, EBV (HHV-4), Human Metapneumo virus, Norovirus, Enterovirus, Smallpox, West Nile Virus, Paramyxoviridae, Rhino virus, Mononucleosis, coxsackievirus B, Influenza, polio, Measles, Rubella, HPV, Zika, Mumps, Herpes viridae, Chikungunya, H. influenzae, and SARS-COV-2 viruses. In some embodiments, the viral protein is from SARS-COV-2. In some embodiments, the viral protein is a SARS-COV-2 spike protein. In some embodiments, the antibody or antigen binding fragment thereof comprises an amino acid sequence having at least 80%, at least 85%, at least 90%, at least 95%, or at least 98% sequence identity to an antibody set forth in Table 3.

In some embodiments, the antibody or antigen binding fragment binds specifically to a cancer antigen. In some embodiments, the antibody or antigen binding fragment thereof binds specifically to a protein or component of a bacteria. In some embodiments, the antibody or antigen binding fragment thereof binds specifically to a protein or component of a parasite. In some embodiments, the antibody or antigen binding fragment thereof binds specifically to an allergen. In some embodiments, the antibody or antigen binding fragment thereof binds specifically to an immune checkpoint molecule. In some embodiments, the antibody or antigen binding fragment thereof binds specifically to an antigen implicated in an inflammatory disease.

In some embodiments, the administering produces a peak blood plasma level of the antibody or antigen binding fragment thereof of at least 75 ng/mL, at least 100 ng/ml, at least 150 ng/mL, at least 200 ng/ml, at least 250 ng/mL, at least 300 ng/mL, at least 400 ng/ml, at least 500 ng/mL, at least 600 ng/mL, at least 700 ng/mL, at least 800 ng/ml, at least 900 ng/ml, or at least 1000 ng/mL. In some embodiments, the administering occurs without electroporation or hydroporation.

In some embodiments, the plasmid is a DNA plasmid.

In another aspect provided herein is a method of inducing antibody production in the subject, comprising administering to the subject a system provided herein. In some embodiments, administration of the plasmid encapsulated in the lipid vesicle to the subject produces a blood plasma level of the antibody or antigen binding fragment thereof of at least 50 ng/mL.

In some embodiments, the administering is performed intramuscularly, subcutaneously, intradermally, intranasally, orally, intrathecally, or intravenously. In some embodiments, the administering is performed intramuscularly. In some embodiments, the administering is performed intravenously. In some embodiments, the administering is performed without electroporation or hydroporation. In some embodiments, the administering produces a peak blood plasma level of the antibody or antigen binding fragment thereof of at least 75 ng/ml, at least 100 ng/ml, at least 150 ng/ml, at least 200 ng/ml, at least 250 ng/ml, at least 300 ng/ml, at least 400 ng/mL, at least 500 ng/mL, at least 600 ng/ml, at least 700 ng/ml, at least 800 ng/mL, at least 900 ng/mL, or at least 1000 ng/mL.

In some embodiments, the administering occurs 1 or 2 times. In some embodiments, the method comprises administering 2 doses of the plasmid to the subject. In some embodiments, the 2 doses are administered intravenously. In some embodiments, the 2 doses are administered from about 2 weeks to about 12 weeks apart. In some embodiments, administration of the second dose results in peak blood plasma level of the antibody or antigen binding fragment which is greater than 2-fold higher than the peak blood plasma level achieved after the first dose. In some embodiments, administration of the second dose results in a peak blood plasma level of the antibody or antigen binding fragment which is at least 3-fold, at least 4-fold, or at least 5-fold higher than the peak blood plasma level achieved after the first dose.

In some embodiments, the administering comprises delivery of from about 0.1 mg/kg to about 20 mg/kg of the plasmid to the subject. In some embodiments, the administering comprises delivery of from about 0.1 mg/kg to about 20 mg/kg of the plasmid to the subject per dose. In some embodiments, the blood plasma level of the antibody or antigen binding fragment is sustained at a concentration of at least 50 ng/mL, at least 100 ng/ml, at least 200 ng/mL, at least 300 ng/mL, at least 400 ng/mL, at least 500 ng/mL, at least 500 ng/ml, at least 600 ng/mL, at least 700 ng/mL, at least 800 ng/ml, at least 900 ng/ml, or at least 1000 ng/ml for a period of at least 4 weeks, at least 6 weeks, at least 8 weeks, at least 10 weeks, or at least 20 weeks after the administration. In some embodiments, the blood plasma level of the antibody or antigen binding fragment is sustained at a concentration of at least 50% of the peak blood plasma concentration achieved for a period of at least 4 weeks, at least 6 weeks, at least 8 weeks, at least 10 weeks, at least 20 weeks, at least 30 weeks, or at least 40 weeks after the administration. In some embodiments, the blood plasma level of the antibody or antigen binding fragment is sustained at a concentration of at least 25% of the peak blood plasma concentration achieved for a period of at least 4 weeks, at least 6 weeks, at least 8 weeks, at least 10 weeks, at least 20 weeks, at least 30 weeks, or at least 40 weeks after the administration. In some embodiments, the blood plasma level of the antibody or antigen binding fragment is sustained at a concentration of at least 10% of the peak blood plasma concentration achieved for a period of at least 4 weeks, at least 6 weeks, at least 8 weeks, at least 10 weeks, at least 20 weeks, at least 30 weeks, or at least 40 weeks after the administration. In some embodiments, the sustained concentration of antibody is achieved after a single administration. In some embodiments, the sustained concentration of antibody is achieved after two administrations.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing, of which:

FIG. 1A shows plasma concentrations of human antibodies in Rag2 knockout mice nine days after administration of a DNA encoded antibody system provided herein. In total 10 mice were transfected with the same protocol (Antibody expression construct, transfection route, and amount of DNA).

FIG. 1B shows plasma concentrations of human antibodies in Rag2 knockout mice 16 days after administration of a DNA encoded antibody system provided herein.

FIG. 1C shows plasma concentrations of human antibodies in Rag2 knockout mice 23 days after administration of a DNA encoded antibody system provided herein.

FIG. 1D shows plasma concentrations of human antibodies in Rag2 knockout mice 30 days after administration of a DNA encoded antibody system provided herein.

FIG. 1E shows plasma concentrations of human antibodies in Rag2 knockout mice 37 days after administration of a DNA encoded antibody system provided herein.

FIG. 1F shows plasma concentrations of human antibodies in Rag2 knockout mice 44 days after administration of a DNA encoded antibody system provided herein.

FIG. 1G shows plasma antibody concentrations for single and dual doses for the indicated antibody formats. For both IV formats, antibody levels increased following the second administration at day 60.

FIG. 1H shows plasma antibody concentrations over time for the indicated dosing regimens.

FIG. 1I shows plasma antibody concentrations over time for the indicated dosing regimens as measured using a commercial IgG1 standard.

FIG. 1J shows plasma antibody concentrations over time for the indicated dosing regiments as measured using an internally generated IgG1 standard, which includes re-measurements of samples displayed in FIG. 1P. Use of the internal standard shows antibody levels which are ˜25-fold lower than the commercial standard.

FIG. 2 shows time course of antibody expression for indicated routes of administration in Rag2 knockout mice.

FIG. 3A shows domain architecture of a SARS-COV-2 spike protein.

FIG. 3B shows a schematic of binding of mAb1 and mAb2 binding to the SARS-COV-2 spike protein receptor biding domain (RBD) at non-overlapping sites.

FIG. 4A shows domain arrangement of monoclonal antibodies, heavy chain only antibodies, and VHH antibodies.

FIG. 4B shows a strand arrangement of a VHH variable region.

FIG. 5A shows a vector map for the expression plasmid of the single transcript T2A formatted construct for mAb1.

FIG. 5B shows a vector map for the expression plasmid encoding the heavy chain of mAb1 for the two plasmid (HC+LC) formatted construct.

FIG. 5C shows a vector map for the expression plasmid encoding the light chain of mAb1 of the two plasmid (HC+LC) formatted construct.

FIG. 6 shows binding to the receptor binding domain of the Wuhan strain of SARS-CoV-2 of antibodies in plasma of Rag2 knockout mice 44 days after administration of a DNA encoded antibody system provided herein.

FIG. 7A shows plasma antibody concentrations for single doses of the indicated antibody formats at the indicated doses calculated using an internally generated human IgG1 standard.

FIG. 7B shows plasma antibody concentrations for some of the same samples in FIG. 7A measured with a more sensitive assay.

FIG. 8 shows plasma antibody concentrations for samples with and without the SV40e element.

FIG. 9 shows plasma antibody concentrations for VHH format antibodies in ng/ml (left) and nM (right).

### DETAILED DESCRIPTION

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment. The section headings used herein are for organizational purposes only and are not to be construed as limiting the subject matter described.

### I. Antibody or Antigen Binding Fragment Expression Systems

Provided herein are systems for expression of antibodies or antigen binding fragments. In some embodiments, the systems are configured to express a therapeutically relevant amount of the antibody or antigen binding fragment when administered to a subject.

In one aspect, provided herein, is a system for expressing an antibody or an antigen binding fragment thereof. In some embodiments, the system is configured to express the antibody or antigen binding fragment thereof when administered to a subject. In some embodiments, the system comprises a plasmid. In some embodiments, the plasmid comprises polynucleotide sequence encoding a heavy chain variable domain of the antibody or an antigen binding fragment thereof. In some embodiments, the plasmid is encapsulated in a lipid vesicle. In some embodiments, the lipid vesicle is administered to a subject. In some embodiments the administered lipid vesicle produces a peak blood plasma level of antibody or antigen binding fragment of at least 50 ng per ml.

In another aspect, provided herein, is a system for expressing an antibody or an antigen binding fragment thereof, wherein the system comprises a vector. In some embodiments, the vector comprises polynucleotide sequence encoding a heavy chain variable domain of the antibody or an antigen binding fragment thereof. In some embodiments, the vector is encapsulated in a lipid vesicle. In some embodiments, the lipid vesicle is administered to a subject. In some embodiments the administered lipid vesicle produces a peak blood plasma level of antibody or antigen binding fragment of at least 50 ng per ml.

In still another aspect, provided herein, is a system for expressing an antibody or an antigen binding fragment thereof in the tissues of a subject. In some embodiments, the system comprises a DNA molecule. In some embodiments, the DNA molecule comprises polynucleotide sequence encoding a heavy chain variable domain of the antibody or an antigen binding fragment thereof. In some embodiments, the DNA molecule is encapsulated in a lipid vesicle. In some embodiments, the lipid vesicle is administered to a subject. In some embodiments the administered lipid vesicle produces a peak blood plasma level of antibody or antigen binding fragment of at least 50 ng per ml.

### Vectors

In one aspect, provided herein, is a vector comprising a polynucleotide sequence encoding an antibody or antigen binding fragment with affinity to a disease associated antigen. In some embodiments herein is a DNA vector comprising a polynucleotide sequence encoding an antibody or antigen binding fragment with affinity to a disease associated antigen. The disease associated antigen may be an antigen associated with a disease, wherein an example of such an antigen is protein or other component of a virus, a bacterium, a parasite, or a cancer, or an antigen implicated in another disease such as an autoimmune disease or inflammatory disorder. In some embodiments, the DNA vector is a plasmid, a viral vector, a cosmid, or an artificial chromosome. In some embodiments, the DNA vector is a plasmid.

In embodiments wherein the vector is a plasmid, it may be advantageous that the plasmid be at or below a certain size. In some embodiments, a smaller plasmid provided the advantage of better loading the vector into a desired formulation (e.g., a proteolipid vehicle as provided herein), as well as enhanced expression due to the lessor potential of cross reactivity owing to a larger size. In some embodiments, the plasmid comprises at most about 50,000 base pairs (bp), at most about 45,000 bp, at most about 40,000 bp, at most about 35,000 bp, at most about 30,000 bp, at most about 25,000 bp, at most about 20,000 bp, at most about 15,000, at most about 10,000, at most about 9,000, at most about 8,000, at most about 7,000, at most about 6,000, at most about 5,000 bp, or at most about 4,000 bp (for double-stranded DNA plasmids). In some embodiments, the plasmid comprises at most about 5,000 bp. In some embodiments, the plasmid comprises at most about 4,000 bp. In some embodiments, the plasmid is between 4,000 bp and 5,000 bp. In some embodiments, the plasmid is between 3,000 bp and 5,000 bp. In some embodiments, the plasmid is between 3,000 bp and 4,000 bp. In some embodiments, the plasmid is between 2,500 bp and 5,000 bp.

In some embodiments, the plasmid is or is derived from a bacterial or fungal plasmid. In some embodiments, the plasmid is or is derived from a yeast plasmid. In some embodiments, the plasmid is or is derived from a bacterium. In some embodiments, the plasmid backbone is derived from a bacterium or a fungus. In some embodiments, the plasmid backbone is derived from a bacterium. In some embodiments, the plasmid backbone is derived from a yeast.

In some embodiments, the plasmid comprises an R6K origin of replication. In some embodiments, the plasmid comprises a 140 bp RNA-based sucrose selectable antibiotic free marker (RNA-OUT). In some embodiments, the plasmid backbone (e.g., the portions of the plasmid not implicated directly in the expression of the encoded gene, such as the encoding sequence, polyadenylation sequence, signal peptide encoding sequence, and promoter(s)) is less than 1000 bp, less than 900 bp, less than 800 bp, less than 700 bp, less than 600 bp, or less than 500 bp. In some embodiments, the plasmid consists essentially of an origin of replication, a selectable marker, and portions of the plasmid directly implicated in the expression of the encoded gene.

In some embodiments, the plasmid backbone is a NTC9385R plasmid. The NTC9835R plasmid is an expression vector that contains a bacterial backbone comprising a 140 bp RNA based sucrose selectable antibiotic free marker (RNA-OUT). The NTC9385R plasmid is described in U.S. Pat. No. 9,550,998, which is hereby incorporated by reference as if set forth herein in its entirety. NTC9835R is sold commercially by Nature Technology Corporation under the trade name Nanoplasmid™.

In some embodiments, the vector contains a polynucleotide sequence encoding a secretion signal peptide. In some embodiments, the polynucleotide encoding the secretion signal peptide is fused in-frame with the 5′ end of a polynucleotide encoding an antibody heavy chain, an antibody heavy chain antigen binding fragment, an antibody light chain, and/or an antibody light chain antigen binding fragment. In some embodiments, the polynucleotide encoding the secretion signal peptide is fused to the heavy chain encoding polynucleotide sequence. In some embodiments, the polynucleotide encoding the secretion signal peptide is fused in-frame with the 5′ end of a therapeutic antibody light chain or light chain antigen binding fragment encoding polynucleotide sequence. In some embodiments, the polynucleotide encoding the secretion signal peptide is fused to the light chain encoding polynucleotide sequence. In some embodiments, the secretion signal peptide is fused to a VHH antibody.

In some embodiments, the vectors encoding an antibody or antigen binding fragment as provided herein comprise one or more promoters which aid in the transcription of the sequences encoding the antibody or antigen binding fragment. In some embodiments, the vector comprises a promoter operably linked to the polynucleotide sequence encoding the antibody or antigen binding fragment. In some embodiments, the promoter allows for enhanced expression of an mRNA transcript for the antibody or antigen binding fragment. In some embodiments, the vector comprises a eukaryotic promoter. In some embodiments, the promoter is selected from a CAG promoter, a cytomegalovirus (CMV) promoter, a human elongation factor-1 alpha (EF1A) promoter, a CBh promoter (see, e.g., Hum Gene Ther. 2011 September; 22 (9): 1143-53. doi: 10.1089/hum.2010.245), a chicken β-actin (CBA) promoter, or a spleen focus forming virus (SFFV) promoter.

In some embodiments, the vector comprises a CAG promoter. In some embodiments, the CAG promoter includes a cytomegalovirus (CMV) early enhancer element, the promoter, the first exon, and the first intron of the chicken β-actin gene, and the splice acceptor of the rabbit β-globin gene.

In some embodiments, the vector comprises one or more enhancers (e.g., alternatively to or in addition to those of the CAG promoter). In some embodiments, the vector comprises an SV40 enhancer (SV40e). In some embodiments, the SV40e is incorporated upstream of the region encoding the antibody or antigen binding fragment thereof. In some embodiments, the SV40e is incorporated upstream of a promoter of the region encoding the antibody or antigen binding fragment thereof. In some embodiments, the SV40e is positioned directly upstream of the promoter. In some embodiments, the SV40e is positioned directly upstream of the CAG promoter. In some embodiments, the SV40e has a sequence having at least about 80%, at least about 85%, at least about 90%, at least about 95% or 100% sequence identity to the sequence TGGTTGCTGACTAATTGAGATGCATGCTTTGCATACTTCTGCCTGCTGGGGAGCC TGGGGACTTTCCACACC (SEQ ID NO: 102).

In some embodiments, the vector includes a woodchuck hepatitis virus post-transcriptional regulator element (WPRE). In some embodiments, the WPRE is positioned downstream of the region encoding the antibody or antigen binding fragment thereof. In some embodiments, WPRE is positioned downstream of the region encoding the antibody or antigen binding fragment thereof but upstream of the poly-adenylation signal. In some embodiments, the WPRE has a sequence having at least about 80%, at least about 85%, at least about 90%, at least about 95% or 100% sequence identity to the sequence AATCAACCTCTGGATTACAAAATTTGTGAAAGATTGACTGGTATTCTTAACTATG TTGCTCCTTTTACGCTATGTGGATACGCTGCTTTAATGCCTTTGTATCATGCTATT GCTTCCCGTATGGCTTTCATTTTCTCCTCCTTGTATAAATCCTGGTTGCTGTCTCTT TATGAGGAGTTGTGGCCCGTTGTCAGGCAACGTGGCGTGGTGTGCACTGTGTTTG CTGACGCAACCCCCACTGGTTGGGGCATTGCCACCACCTGTCAGCTCCTTTCCGG GACTTTCGCTTTCCCCCTCCCTATTGCCACGGCGGAACTCATCGCCGCCTGCCTTG CCCGCTGCTGGACAGGGGCTCGGCTGTTGGGCACTGACAATTCCGTGGTGTTGTC GGGGAAGCTGACGTCCTTTCCATGGCTGCTCGCCTGTGTTGCCACCTGGATTCTG CGCGGGACGTCCTTCTGCTACGTCCCTTCGGCCCTCAATCCAGCGGACCTTCCTTC CCGCGGCCTGCTGCCGGCTCTGCGGCCTCTTCCGCGTCTTCGCCTTCGCCCTCAGA CGAGTCGGATCTCCCTTTGGGCCGCCTCCCCGC (SEQ ID NO: 103).

### Single Transcript Expression Vector (e.g., Plasmids) for Light Chain and Heavy Chain Antibodies

In some embodiments, a system for expressing an antibody or antigen binding fragment as provided herein comprises a single vector (e.g., a DNA plasmid) which encodes both a heavy chain of an antibody, or a fragment thereof, and a light chain of an antibody, or a fragment thereof. In some embodiments, both the heavy chain, or the fragment thereof, and the light chain, or the fragment thereof, are encoded such that both chains or fragments thereof are transcribed in a single transcript, thus yield expression of both chains or fragments thereof at the same time in the same cell, and in the same concentration. An exemplary vector showing such a construct is shown in FIG. 5A.

In some embodiments, the polynucleotide sequences encoding the heavy chain and light chain of the antibody, or antigen binding fragments thereof, are configured to be read as a single transcript. In some embodiments, the encoded fused antibody heavy and light chains are separated by a self-cleavage peptide encoding sequence. In some embodiments, the cleavage peptide encoded is a Furin-T2A self-cleavage sequence. In some embodiments, the Furin-T2A cleavage peptide sequence is RRKRGSGEGRGSLLTCGDVEENPGP (SEQ ID NO: 104). In some embodiments, the Furin-T2A cleavage peptide sequence has at least 75%, at least 80%, at least 85%, at least 90%, or at least 95% identity with the peptide sequence RRKRGSGEGRGSLLTCGDVEENPGP (SEQ ID NO: 104). In some embodiments, these polypeptides transit a vesicular membrane, assemble in the luminal space of the exocytic vesicular system and are secreted.

In some embodiments, the heavy chain or fragment thereof of the antibody comprises a variable heavy chain domain and a CH1 domain. In some embodiments, the heavy chain or fragment thereof further comprises a CH2 domain, a CH3 domain, or both. In some embodiments, the light chain or fragment thereof of the antibody comprises a variable light chain domain and a constant light chain domain.

### Split Antibody Expression System

In some embodiments, a system for expressing an antibody or antigen binding fragment in a subject as provided herein is configured to produce the antibody or antigen binding fragment through the translation of two separate transcripts. In some embodiments, a heavy chain of the antibody, or a fragment thereof, and a light chain of the antibody, or a fragment thereof are encoded on one or more vectors (e.g., plasmids) such that each is separately transcribed. In some embodiments, the heavy chain of the antibody or fragment thereof and the light chain of the antibody or fragment thereof are encoded on separate vectors. In some embodiments, the separate plasmids are formulated together such that the vectors can be delivered to the same cell (e.g., both encapsulated in the same lipid vesicle). In some embodiments, both the heavy chain or fragment thereof and light chain or fragment thereof are expressed within the same cell. In some embodiments, the heavy chain or fragment thereof and the light chain or fragment thereof are expressed in different cells. Exemplary DNA plasmid vectors separately encoding a heavy chain and a light chain of an antibody are shown in FIG. 5B and FIG. 5C. The vectors depicted therein have substantially identical non-coding portions as compared to the vector depicted in FIG. 5A (i.e., only the coding region is changed).

In some embodiments, equimass ratios of the vector encoding the heavy chain or fragment thereof of the antibody and the vector encoding the light chain or fragment thereof are used in a system as provided herein. In some embodiments, equimolar ratios of the vector encoding the heavy chain or fragment thereof of the antibody and the vector encoding the light chain or fragment thereof are used in a system as provided herein. In some embodiments, the molar ratio of vector encoding the heavy chain or fragment thereof to the vector encoding the light chain or fragment thereof is from about 2:1 to about 1:2. In some embodiments, the molar ratio of vector encoding the heavy chain or fragment thereof to the vector encoding the light chain or fragment thereof is about 2:1, about 1.9:1, about 1.8:1, about 1.7:1, about 1.6:1, about 1.5:1, about 1.4:1, about 1.3:1, about 1.2:1, about 1.1:1, about 1:1, about 1:1.1, about 1:1.2, about 1:1.3, about 1:1.4, about 1:1.5, about 1:1.6, about 1:1.7, about 1:1.8, about 1:1.9, or about 1:2. In some embodiments, the molar ratio of vector encoding the heavy chain or fragment thereof to the vector encoding the light chain or fragment thereof is from about 1.5:1 to about 2:1. In some embodiments, the molar ratio of vector encoding the heavy chain or fragment thereof to the vector encoding the light chain or fragment thereof is from about 1.5:1 to about 2:1. In some embodiments, the molar ratio of vector encoding the heavy chain or fragment thereof to the vector encoding the light chain or fragment thereof is from about 1.6:1 to about 1.8:1. In some embodiments, the molar ratio of vector encoding the heavy chain or fragment thereof to the vector encoding the light chain or fragment thereof is about 1.7:1.

### V_{H}H Expression

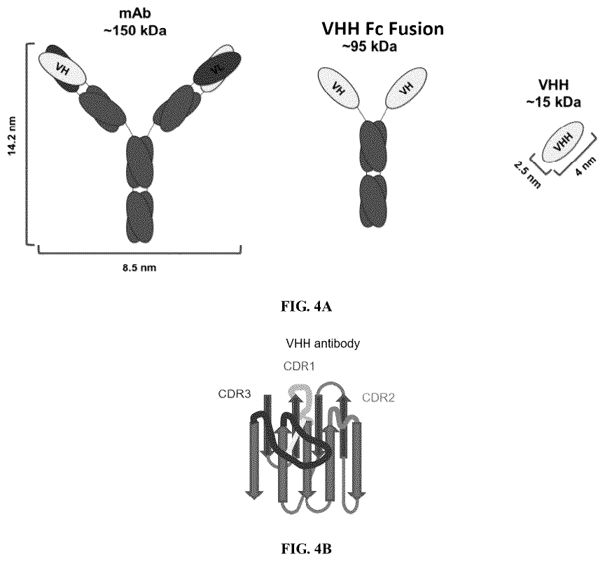

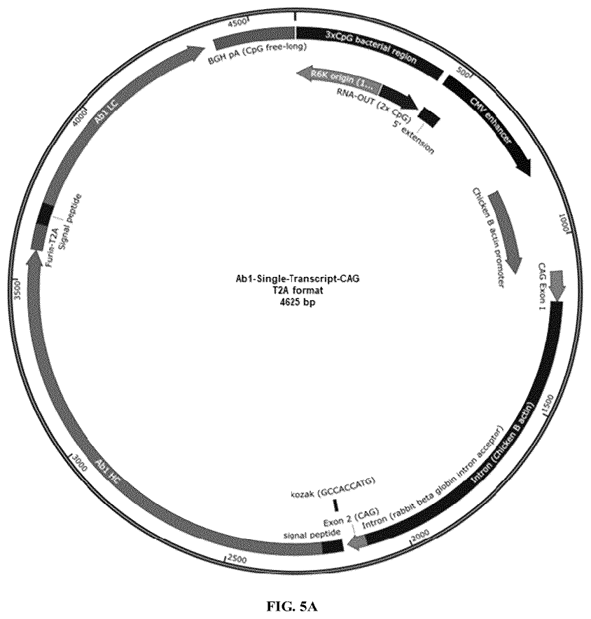

In some embodiments, a system for expressing an antibody or antigen binding fragment as provided herein comprises a vector which encodes a heavy chain variable domain of the antibody or antigen binding fragment thereof. In some embodiments, the vector does not encode an entire antibody heavy chain. In some embodiments, the vector encodes a V_{H}H antibody. An exemplary cartoon depiction of a V_{H}H antibody structure is shown in FIG. 4B. In some embodiments, the vector encodes a V_{H}H fused to an Fc region. An exemplary depiction of a V_{H}H fused to an Fc region is shown in FIG. 4A (middle), along with a depiction of a full antibody (left) and V_{H}H alone (right). In some embodiments, the vector encodes a V_{H}H fused to an Fc region through a linker peptide. In some embodiments, the vector encodes a V_{H}H fused to an Fc region through a hinge region or a modified hinge region. In some embodiments, the antibody is a camelized antibody which only contains a heavy chain. In some embodiments, the vector encodes only the variable domains of a heavy chain to produce a single chain V_{H}H antibody.

### Antibodies and Antigen Binding Fragments

In some embodiments, an antibody or antigen binding fragment of the disclosure specifically binds to a target antigen. An antibody or antigen binding fragment selectively binds or preferentially binds to a target if it binds with greater affinity, avidity, more readily, and/or with greater duration than it binds to other substances. As such, “specific binding” or “preferential binding” does not necessarily require (although it can include) exclusive binding. Generally, but not necessarily, reference to specific binding means preferential binding where the affinity of the antibody, or antigen binding fragment thereof, is at least at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody for an unrelated substance.

As used herein, the term “antibody” refers to an immunoglobulin (Ig), polypeptide, or a protein having a binding domain which is, or is homologous to, an antigen binding domain. The term further includes “antigen binding fragments” and other interchangeable terms for similar binding fragments as described below. Native antibodies and native immunoglobulins (Igs) are generally heterotetrameric glycoproteins of about 150,000 Daltons, composed of two identical light chains and two identical heavy chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“VH”) followed by a number of constant domains (“CH”). Each light chain has a variable domain at one end (“VL”) and a constant domain (“CL”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

In some instances, an antibody or an antigen binding fragment comprises an isolated antibody or antigen binding fragment, a purified antibody or antigen binding fragment, a recombinant antibody or antigen binding fragment, a modified antibody or antigen binding fragment, or a synthetic antibody or antigen binding fragment.

Antibodies and antigen binding fragments herein can be partly or wholly synthetically produced. An antibody or antigen binding fragment can be a polypeptide or protein having a binding domain which can be, or can be homologous to, an antigen binding domain. In one instance, an antibody or an antigen binding fragment can be produced in an appropriate in vivo animal model and then isolated and/or purified.

Depending on the amino acid sequence of the constant domain of its heavy chains, immunoglobulins (Igs) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. An Ig or portion thereof can, in some cases, be a human Ig. In some instances, a CH3 domain can be from an immunoglobulin. In some cases, a chain or a part of an antibody or antigen binding fragment, a modified antibody or antigen binding fragment, or a binding agent can be from an Ig. In such cases, an Ig can be IgG, an IgA, an IgD, an IgE, or an IgM, or is derived therefrom. In cases where the Ig is an IgG, it can be a subtype of IgG, wherein subtypes of IgG can include IgG1, an IgG2a, an IgG2b, an IgG3, or an IgG4. In some cases, a CH3 domain can be from an immunoglobulin selected from the group consisting of an IgG, an IgA, an IgD, an IgE, and an IgM, or derived therefrom. In some embodiments, an antibody or antigen binding fragment described herein comprises an IgG or is derived therefrom. In some instances, an antibody or antigen binding fragment comprises an IgG1 or is derived therefrom. In some instances, an antibody or antigen binding fragment comprises an IgG4 or is derived therefrom. In some embodiments, an antibody or antigen binding fragment described herein comprises an IgM, is derived therefrom, or is a monomeric form of IgM. In some embodiments, an antibody or antigen binding fragment described herein comprises an IgE or is derived therefrom. In some embodiments, an antibody or antigen binding fragment described herein comprises an IgD or is derived therefrom. In some embodiments, an antibody or antigen binding fragment described herein comprises an IgA or is derived therefrom.

The “light chains” of antibodies (immunoglobulins) from any vertebrate species can be assigned to one of two clearly distinct types, called kappa (“K” or “K”) or lambda (“2”), based on the amino acid sequences of their constant domains. In some embodiments, the antibody or antigen binding fragment comprises a kappa light chain. In some embodiments, the antibody or antigen binding fragment comprises a lambda light chain.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (e.g., Kabat et al., Sequences of Proteins of Immunological Interest, (5th Ed., 1991, National Institutes of Health, Bethesda Md. (1991), pages 647-669; hereafter “Kabat”); and (2) an approach based on crystallographic studies of antigen-antibody complexes (Al-Iazikani et al. (1997) J. Molec. Biol. 273:927-948)). As used herein, a CDR may refer to CDRs defined by either approach or by a combination of both approaches.

With respect to antibodies, the term “variable domain” refers to the variable domains of antibodies that are used in the binding and specificity of each particular antibody for its particular antigen. However, the variability is not evenly distributed throughout the variable domains of antibodies. Rather, it is concentrated in three segments called hypervariable regions (also known as CDRs) in both the light chain and the heavy chain variable domains. More highly conserved portions of variable domains are called the “framework regions” or “FRs.” The variable domains of unmodified heavy and light chains each contain four FRs (FR1, FR2, FR3, and FR4), largely adopting a β-sheet configuration interspersed with three CDRs which form loops connecting and, in some cases, part of the β-sheet structure. The CDRs in each chain are held together in close proximity by the FRs and, with the CDRs from the other chain, contribute to the formation of the antigen binding site of antibodies (see, Kabat).

The terms “hypervariable region” and “CDR” when used herein, refer to the amino acid residues of an antibody which are responsible for antigen binding. The CDRs comprise amino acid residues from three sequence regions which bind in a complementary manner to an antigen and are known as CDR1, CDR2, and CDR3 for each of the VH and VL chains. In the light chain variable domain, the CDRs typically correspond to approximately residues 24-34 (LCDR1), 50-56 (LCDR2), and 89-97 (LCDR3), and in the heavy chain variable domain the CDRs typically correspond to approximately residues 31-35 (HCDR1), 50-65 (HCDR2), and 95-102 (HCDR3) according to Kabat. It is understood that the CDRs of different antibodies may contain insertions, thus the amino acid numbering may differ. The Kabat numbering system accounts for such insertions with a numbering scheme that utilizes letters attached to specific residues (e.g., 27A, 27B, 27C, 27D, 27E, and 27F of CDRLI in the light chain) to reflect any insertions in the numberings between different antibodies. Alternatively, in the light chain variable domain, the CDRs typically correspond to approximately residues 26-32 (LCDR1), 50-52 (LCDR2), and 91-96 (LCDR3), and in the heavy chain variable domain, the CDRs typically correspond to approximately residues 26-32 (HCDR1), 53-55 (HCDR2), and 96-101 (HCDR3) according to Chothia and Lesk (J. Mol. Biol., 196:901-917 (1987)).

As used herein, “framework region,” “FW,” or “FR” refers to framework amino acid residues that form a part of the antigen binding pocket or groove. In some embodiments, the framework residues form a loop that is a part of the antigen binding pocket or groove and the amino acids residues in the loop may or may not contact the antigen. Framework regions generally comprise the regions between the CDRs. In the light chain variable domain, the FRs typically correspond to approximately residues 0-23 (LFR1), 35-49 (LFR2), 57-88 (LFR3), and 98-109 and in the heavy chain variable domain the FRs typically correspond to approximately residues 0-30 (HFR1), 36-49 (HFR2), 66-94 (HFR3), and 103-133 according to Kabat. As discussed above with the Kabat numbering for the light chain, the heavy chain too accounts for insertions in a similar manner (e.g., 35A, 35B of HCDR1 in the heavy chain). Alternatively, in the light chain variable domain, the FRs typically correspond to approximately residues 0-25 (LFR1), 33-49 (LFR2) 53-90 (LFR3), and 97-109 (LFR4), and in the heavy chain variable domain, the FRs typically correspond to approximately residues 0-25 (HFR1), 33-52 (HFR2), 56-95 (HFR3), and 102-113 (HFR4) according to Chothia and Lesk, Id. The loop amino acids of a FR can be assessed and determined by inspection of the three-dimensional structure of an antibody heavy chain and/or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g., structural positions) are, generally, less diversified. The three-dimensional structure of the antibody variable domain can be derived from a crystal structure or protein modeling.

In the present disclosure, the following abbreviations (in the parentheses) are used in accordance with the customs, as necessary: heavy chain variable region (HCVR), light chain variable region (LCVR), complementarity determining region (CDR), first complementarity determining region (CDR1), second complementarity determining region (CDR2), third complementarity determining region (CDR3), heavy chain first complementarity determining region (HCDR1), heavy chain second complementarity determining region (HCDR2), heavy chain third complementarity determining region (HCDR3), light chain first complementarity determining region (LCDR1), light chain second complementarity determining region (LCDR2), and light chain third complementarity determining region (LCDR3).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is generally defined to stretch from an amino acid residue at position Cys226, or from Pro230, to the carboxyl-terminus thereof. The numbering of the residues in the Fc region is that of the EU index as in Kabat. The Fc region of an immunoglobulin generally comprises two constant domains, CH2 and CH3.

“Antibodies” useful in the present disclosure encompass, but are not limited to, monoclonal antibodies, polyclonal antibodies, chimeric antibodies, bispecific antibodies, multispecific antibodies, heteroconjugate antibodies, humanized antibodies, human antibodies, grafted antibodies, deimmunized antibodies, mutants thereof, fusions thereof, immunoconjugates thereof, antigen binding fragments thereof, and/or any other modified configuration of the immunoglobulin molecule that comprises an antigen recognition site of the required specificity, including glycosylation variants of antibodies, amino acid sequence variants of antibodies, and covalently modified antibodies.

In some instances, an antibody is a monoclonal antibody. As used herein, a “monoclonal antibody” refers to an antibody obtained from a population of substantially homogeneous antibodies, i.e., the individual antibodies comprising the population are identical except for possible naturally-occurring mutations that may be present in minor amounts. In contrast to polyclonal antibody preparations, which typically include different antibodies directed against different determinants (epitopes), each monoclonal antibody is directed against a single determinant on the antigen (epitope). The modifier “monoclonal” indicates the character of the antibody as being obtained from a substantially homogeneous population of antibodies and is not to be construed as requiring production of the antibody by any particular method.

In some instances, an antibody is a humanized antibody. As used herein, “humanized” antibodies refer to forms of non-human (e.g., murine) antibodies that are specific chimeric immunoglobulins, immunoglobulin chains, or fragments thereof that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences but are included to further refine and optimize antibody performance. In general, a humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in, for example, WO 99/58572. Other forms of humanized antibodies have one or more CDRs (one, two, three, four, five, or six) which are altered with respect to the original antibody, which are also termed one or more CDRs “derived from” one or more CDRs from the original antibody.

If needed, an antibody or an antigen binding fragment described herein can be assessed for immunogenicity and, as needed, be deimmunized (i.e., the antibody is made less immunoreactive by altering one or more T cell epitopes). As used herein, a “deimmunized antibody” means that one or more T cell epitopes in an antibody sequence have been modified such that a T cell response after administration of the antibody to a subject is reduced compared to an antibody that has not been deimmunized. Analysis of immunogenicity and T-cell epitopes present in the antibodies and antigen binding fragments described herein can be carried out via the use of software and specific databases. Exemplary software and databases include iTope™ developed by Antitope of Cambridge, England. iTope™, is an in silico technology for analysis of peptide binding to human MHC class II alleles. The iTope™ software predicts peptide binding to human MHC class II alleles and thereby provides an initial screen for the location of such “potential T cell epitopes.” iTope™ software predicts favorable interactions between amino acid side chains of a peptide and specific binding pockets within the binding grooves of 34 human MHC class II alleles. The location of key binding residues is achieved by the in silico generation of 9mer peptides that overlap by one amino acid spanning the test antibody variable region sequence. Each 9mer peptide can be tested against each of the 34 MHC class II allotypes and scored based on their potential “fit” and interactions with the MHC class II binding groove. Peptides that produce a high mean binding score (>0.55 in the iTope™ scoring function) against >50% of the MHC class II alleles are considered as potential T cell epitopes. In such regions, the core 9 amino acid sequence for peptide binding within the MHC class II groove is analyzed to determine the MHC class II pocket residues (P1, P4, P6, P7, and P9) and the possible T cell receptor (TCR) contact residues (P-1, P2, P3, P5, P8). After identification of any T-cell epitopes, amino acid residue changes, substitutions, additions, and/or deletions can be introduced to remove the identified T-cell epitope. Such changes can be made so as to preserve antibody structure and function while still removing the identified epitope. Exemplary changes can include, but are not limited to, conservative amino acid changes.

An antibody can be a human antibody. As used herein, a “human antibody” means an antibody having an amino acid sequence corresponding to that of an antibody produced by a human and/or that has been made using any suitable technique for making human antibodies. This definition of a human antibody includes antibodies comprising at least one human heavy chain polypeptide or at least one human light chain polypeptide. One such example is an antibody comprising murine light chain and human heavy chain polypeptides. In one embodiment, the human antibody is selected from a phage library, where that phage library expresses human antibodies. Human antibodies can also be made by introducing human immunoglobulin loci into transgenic animals, e.g., mice in which the endogenous immunoglobulin genes have been partially or completely inactivated. Alternatively, the human antibody may be prepared by immortalizing human B lymphocytes that produce an antibody directed against a target antigen (such B lymphocytes may be recovered from an individual or may have been immunized in vitro).

Any of the antibodies herein can be bispecific. Bispecific antibodies are antibodies that have binding specificities for at least two different antigens and can be prepared using the antibodies disclosed herein. Traditionally, the recombinant production of bispecific antibodies was based on the co-expression of two immunoglobulin heavy chain-light chain pairs, with the two heavy chains having different specificities. Bispecific antibodies can be composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations.

According to one approach to making bispecific antibodies, antibody variable domains with the desired binding specificities (antibody-antigen combining sites) are fused to immunoglobulin constant domain sequences. The fusion can be with an immunoglobulin heavy chain constant domain, comprising at least part of the hinge, CH2 and CH3 regions. The first heavy chain constant region (CH1), containing the site necessary for light chain binding, can be present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some instances, an antibody herein is a chimeric antibody. “Chimeric” forms of non-human (e.g., murine) antibodies include chimeric antibodies which contain minimal sequence derived from a non-human Ig. For the most part, chimeric antibodies are murine antibodies in which at least a portion of an immunoglobulin constant region (Fc), typically that of a human immunoglobulin, is inserted in place of the murine Fc.

Provided herein are antibodies and antigen binding fragments thereof, modified antibodies and antigen binding fragments thereof, and binding agents that specifically bind to one or more epitopes on one or more target antigens. In one instance, a binding agent selectively binds to an epitope on a single antigen. In another instance, a binding agent is bivalent and either selectively binds to two distinct epitopes on a single antigen or binds to two distinct epitopes on two distinct antigens. In another instance, a binding agent is multivalent (i.e., trivalent, quadrivalent, etc.) and the binding agent binds to three or more distinct epitopes on a single antigen or binds to three or more distinct epitopes on two or more (multiple) antigens.

Antigen binding fragments of any of the antibodies herein are also contemplated. The terms “antigen binding portion of an antibody,” “antigen binding fragment,” “antigen binding domain,” “antibody fragment,” or a “functional fragment of an antibody” are used interchangeably herein to refer to one or more fragments of an antibody that retain the ability to specifically bind to an antigen. Representative antigen binding fragments include, but are not limited to, a Fab, a Fab′, a F(ab′)2, a bispecific F(ab′)2, a trispecific F(ab′)2, a variable fragment (Fv), a single chain variable fragment (scFv), a dsFv, a bispecific scFv, a variable heavy domain, a variable light domain, a variable NAR domain, bispecific scFv, an AVIMER®, a minibody, a diabody, a bispecific diabody, triabody, a tetrabody, a minibody, a maxibody, a camelid, a V_{H}H, a minibody, an intrabody, fusion proteins comprising an antibody portion (e.g., a domain antibody), a single chain binding polypeptide, a scFv-Fc, a Fab-Fc, a bispecific T cell engager (BiTE; two scFvs produced as a single polypeptide chain, where each scFv comprises an amino acid sequences a combination of CDRs or a combination of VL/VL described herein), a tetravalent tandem diabody (TandAb; an antibody fragment that is produced as a non-covalent homodimer folder in a head-to-tail arrangement, e.g., a TandAb comprising an scFv, where the scFv comprises an amino acid sequences a combination of CDRs or a combination of VL/VL described herein), a Dual-Affinity Re-targeting Antibody (DART; different scFvs joined by a stabilizing interchain disulphide bond), a bispecific antibody (bscAb; two single-chain Fv fragments joined via a glycine-serine linker), a single domain antibody (sdAb), a fusion protein, a bispecific disulfide-stabilized Fv antibody fragment (dsFv-dsFv′: two different disulfide-stabilized Fv antibody fragments connected by flexible linker peptides).

As used herein, the term “avidity” refers to the resistance of a complex of two or more agents to dissociation after dilution. Apparent affinities can be determined by methods such as an enzyme-linked immunosorbent assay (ELISA) or any other suitable technique. Avidities can be determined by methods such as a Scatchard analysis or any other suitable technique.

As used herein, the term “affinity” refers to the equilibrium constant for the reversible binding of two agents and is expressed as KD. The binding affinity (KD) of an antibody or antigen binding fragment herein can be less than 500 nM, 475 nM, 450 nM, 425 nM, 400 nM, 375 nM, 350 nM, 325 nM, 300 nM, 275 nM, 250 nM, 225 nM, 200 nM, 175 nM, 150 nM, 125 nM, 100 nM, 90 nM, 80 nM, 70 nM, 50 nM, 50 nM, 49 nM, 48 nM, 47 nM, 46 nM, 45 nM, 44 nM, 43 nM, 42 nM, 41 nM, 40 nM, 39 nM, 38 nM, 37 nM, 36 nM, 35 nM, 34 nM, 33 nM, 32 nM, 31 nM, 30 nM, 29 nM, 28 nM, 27 nM, 26 nM, 25 nM, 24 nM, 23 nM, 22 nM, 21 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM, 990 pM, 980 pM, 970 pM, 960 pM, 950 pM, 940 pM, 930 pM, 920 pM, 910 pM, 900 pM, 890 pM, 880 pM, 870 pM, 860 pM, 850 pM, 840 pM, 830 pM, 820 pM, 810 pM, 800 pM, 790 pM, 780 pM, 770 pM, 760 pM, 750 pM, 740 pM, 730 pM, 720 pM, 710 pM, 700 pM, 690 pM, 680 pM, 670 pM, 660 pM, 650 pM, 640 pM, 630 pM, 620 pM, 610 pM, 600 pM, 590 pM, 580 pM, 570 pM, 560 pM, 550 pM, 540 pM, 530 pM, 520 pM, 510 pM, 500 pM, 490 pM, 480 pM, 470 pM, 460 pM, 450 pM, 440 pM, 430 pM, 420 pM, 410 pM, 400 pM, 390 pM, 380 pM, 370 pM, 360 pM, 350 pM, 340 pM, 330 pM, 320 pM, 310 pM, 300 pM, 290 pM, 280 pM, 270 pM, 260 pM, 250 pM, 240 pM, 230 pM, 220 pM, 210 pM, 200 pM, 190 pM, 180 pM, 170 pM, or any integer therebetween. Binding affinity may be determined using surface plasmon resonance (SPR), KINEXA® Biosensor, scintillation proximity assays, isothermal titration calorimetry (ITC) assays, enzyme linked immunosorbent assay (ELISA), ORIGEN immunoassay (IGEN), fluorescence quenching, fluorescence transfer, yeast display, or any combination thereof. Binding affinity may also be screened using a suitable bioassay.

In some embodiments, the antibody or antigen binding fragment comprises an gG1, IgG2a, IgG2b, IgG3, IgG4, IgD, IgM, IgA1, IgA2 or IgE heavy chain, or a portion thereof. In some embodiments, the antibody or antigen binding fragment thereof comprises an IgG1, IgG2a, IgG2b, IgG3, or IgG4 heavy chain, or a portion thereof. In some embodiments, the antibody comprises an IgG1 heavy chain, or a portion thereof.

In some embodiments, the antibody or antigen binding fragment thereof comprises a modified Fc domain. In some embodiments, the modified Fc domain comprises one or more amino acid substitutions relative to a wild type Fc domain of an antibody of the relevant subtype or isotype. In some embodiments, the modified Fc domain has an amino acid sequence at least about 80%, at least about 85%, at least about 90%, at least about 95%, at least about 96%, at least about 97%, at least about 98%, or at least about 99% identical to that of a wild type Fc domain of a corresponding antibody or antigen binding fragment. In some embodiments, the modified Fc domain comprises 1, 2, 3, 4, 5, 6, 7, 8, 9, 10, or more substitution relative to the corresponding wild type Fc domain of the antibody or antigen binding fragment. In some embodiments, the substitutions in the Fc domain allow for the antibody or antigen binding fragment to have desired physicochemical properties, such as enhanced half-life or stability in vivo or other pharmacokinetic parameter, altered binding to Fc receptors, altered glycosylation patterns, to introduce additional disulfide bonds or to disrupt one or more disulfide bonds, or to alter intra- or inter-molecular interactions of the antibody or antigen binding fragment. In some embodiments, an Fc domain of an antibody or antigen binding fragment as provided herein (e.g., an IgG1 heavy chain) comprises one or more substitutions or combinations of substitutions selected from T250Q/M428L; M252Y/S254T/T256E+H433K/N434F; E233P/L234V/L235A/G236A+A327G/A330S/P331S; E333A; S239D/A330L/1332E; P257I/Q311; K326W/E333S; S239D/1332E/G236A; N297A; L234A/L235A; N297A+M252Y/S254T/T256E; K322A and K444A (EU numbering). In some embodiments, the antibody or antigen binding fragment comprises an Fc domain having one or more mutations or combinations of mutations selected from Arg435His (His435), Asn434Ala (A), Met428Leu/Asn434Ser (LS), Thr252Leu/Thr253Ser/Thr254Phe (LSF), Glu294delta/Thr307Pro/Asn434Tyr (C6A-66), Thr256Asn/Ala378Val/Ser383 Asn/Asn434Tyr (C6A-78), and Glu294delta (Del), wherein residue position number is based on EU numbering convention. In some embodiments, the Fc domain comprises one or more substitutions selected from M252Y, S254T, and T256E (EU numbering). In some embodiments, the Fc domain comprises the substitutions M252Y, S254T, and T256E (EU numbering).

In some embodiments, the antibody or antigen binding fragment is a V_{H}H antibody. In some embodiments, the antibody or antigen binding fragment comprises a heavy chain variable domain of a camelid antibody. In some embodiments, the heavy chain variable domain comprises a sequence having at least 80% sequence identity to the sequence AQVQLVETGGGLVQPGGSLRLSCAASXXXXXXXXXMNWVRQAPGKGPEWVSXXX XXXXXXXYTDSVKGRFTISRDNAKNTLYLQMNNLKPEDTALYYCXXXXXXXXXX XRGQGTQVTVSS (SEQ ID NO: 101), wherein each X is independently absent or any amino acid. In some embodiments, the heavy chain variable domain comprises a sequence having at least 80%, at least 85%, at least 90%, at least 95%, at least 98%, or at least 99%, or 100% sequence identity to the sequence AQVQLVETGGGLVQPGGSLRLSCAASXXXXXXXXXMNWVRQAPGKGPEWVSXXX XXXXXXXYTDSVKGRFTISRDNAKNTLYLQMNNLKPEDTALYYCXXXXXXXXXX XRGQGTQVTVSS (SEQ ID NO: 101), wherein each X is independently absent or any amino acid.

In some embodiments, the heavy chain variable domain of the V_{H}H antibody comprises an amino acid sequence having at least 80%, at least 85%, at least 90%, at least 95%, at least 98%, at least 99%, or 100% identity to the V_{H}H variable domain sequence of Ty1, N3113V, or N3130V (as set forth in Table 17).

In some embodiments, the antibody or antigen binding fragment is a V_{H}H fusion protein. In some embodiments, the antibody or antigen binding fragment is a V_{H}H domain fused to an Fc domain. In some embodiments, the V_{H}H domain is fused to an IgG1 Fc domain. In some embodiments, the V_{H}H domain is fused to the Fc domain through a linker peptide. In some embodiments, the linker peptide is an antibody hinge region peptide, or a variant thereof. In some embodiments, the linker peptide comprises an amino acid sequence having at least 80% or at least 90% sequence identity to the sequence SDKTHTCP (SEQ ID NO: 105). In some embodiments, Fc domain comprises a CH2 domain, a CH3 domain, or both. In some embodiments, the Fc domain comprises an amino acid sequence having at least about 80%, at least about 85%, at least about 90%, at least about 95%, at least about 96%, at least about 97%, at least about 98%, at least about 99%, or 100% sequence identity to the sequence PCPAPELLGGPSVFLFPPKPKDTLYITREPEVTCVVVDVSHEDPEVKFNWYVDGVEV HNAKTKPREEQYNSTYRVVSVLTVLHQDWLNGKEYKCKVSNKALPAPIEKTISKAK GQPREPQVYTLPPSRDELTKNQVSLTCLVKGFYPSDIAVEWESNGQPENNYKTTPPV LDSDGSFFLYSKLTVDKSRWQQGNVFSCSVMHEALHNHYTQKSLSLSPGK (SEQ ID NO: 106). In some embodiments, the V_{H}H domain is fused to a modified hinge region and Fc domain comprising an amino acid sequence having at least about 80%, at least about 85%, at least about 90%, at least about 95%, at least about 96%, at least about 97%, at least about 98%, at least about 99%, or 100% identical to the sequence SDKTHTCPPCPAPELLGGPSVFLFPPKPKDTLYITREPEVTCVVVDVSHEDPEVKFNW YVDGVEVHNAKTKPREEQYNSTYRVVSVLTVLHQDWLNGKEYKCKVSNKALPAPI EKTISKAKGQPREPQVYTLPPSRDELTKNQVSLTCLVKGFYPSDIAVEWESNGQPEN NYKTTPPVLDSDGSFFLYSKLTVDKSRWQQGNVFSCSVMHEALHNHYTQKSLSLSP GK (SEQ ID NO: 106).

### Antigens Bound by Antibodies or Antigen Binding Fragments

In some instances, the antibodies or antigen binding fragments encoded by the systems provided herein are useful for the treatment, prevention, or mitigation of one or more diseases associated with an antigen bound by the antibody or antigen binding fragment. In some embodiments, the antibody or antigen binding fragments binds to a disease-associated antigen.

### Infectious Diseases

In some embodiments, the systems for producing antibodies or antigen binding fragments in a subject provided herein are useful for the treatment, management, or prevention of an infectious disease. Infectious diseases include without limitation viral infections, microbial infections, bacterial infections, parasitic infections, fungal infections, and the like. In some embodiments, the systems provided herein are effective for the prophylaxis of an acute infection (e.g., reducing the risk of becoming infected by an agent, such as a virus or bacteria, by a certain amount, such as at least 50%, at least 60%, at least 70%, at least 80%, or at least 90%, or eliminating the risk of becoming infected by an agent). In some embodiments, the systems provided herein are useful in the treatment or management of a chronic infection (e.g., the systems eliminate or reduce one or more symptoms associated with an existing infection, or the systems reduce the prevalence or frequency of such symptoms where the symptoms recur from time to time).

In some embodiments, a system provided herein comprising a vector encoding an antibody or antigen binding fragment which binds to an infectious disease associated antigen is administered as a prophylaxis (e.g., before a subject is infected with the disease-causing agent). In some embodiments, a system provided herein comprising a vector encoding an antibody or antigen binding fragment which binds to an infectious disease associated antigen is administered for treatment of the infection (e.g., treatment of an acute infection shortly after becoming infected or displaying symptoms, or treatment of a chronic infection at a later time period after becoming infected or displaying symptoms).

### Viral Infection

In some embodiments, the vectors of the systems provided herein encode antibodies or antigen binding fragments which bind to an antigen associated with a virus. In some embodiments, the system is effective to induce protection against infection by the virus. In some embodiments, the system is effective to mitigate, reduce, or eliminate infection of the virus. In some embodiments, the antigen associated with the virus a component of the virus. In some embodiments, the antigen associated with the virus is a viral protein, a viral glycan, a viral lipid membrane, or other component. In some embodiments, the antigen associated with the virus is a viral protein.

In some embodiments, the virus for which the antibody or antigen binding fragment is targeted is selected from a group consisting a parvovirus, a picornavirus, a rhabdovirus, a paramyxovirus, an orthomyxovirus, a bunyavirus, a calicivirus, an arenavirus, a polyomavirus, a reovirus, a togavirus, a bunyavirus, a herpes simplex virus, a poxvirus, an adenovirus, a coxsackievirus, a flavivirus, a coronavirus, an astrovirus, an enterovirus, a rotavirus, a norovirus, a retrovirus, a papilloma virus, a parvovirus, an influenza virus, a hemorrhagic fever virus, and a rhinovirus. In some embodiments, the virus for which the antibody or antigen binding fragment is targeted is selected from a group consisting of Hantavirus, Rabies, Nipah, Hendra, Rift Valley Fever, Lassa, Marburg, Crimean Congo Fever, hMPV, RSV, Hepatitis A, Hepatitis B, Hepatitis C, Hepatitis D, Hepatitis E, Norovirus, Monkeypox, Coxpox, Japanese Encephalitis, Yellow Fever, HSV-1, HSV-2, MERS, ChickenPox, Hand, Foot and Mouth, CMV (HHV-5), Equine Encephalitis, EBV (HHV-4), Human Metapneumo virus, Norovirus, Enterovirus, Smallpox, West Nile Virus, Paramyxoviridae, Rhino virus, Mononucleosis, coxsackievirus B, Influenza, polio, Measles, Rubella, HPV, Zika, Mumps, Herpes viridae, Chikungunya, H. influenzae, and SARS-COV-2 viruses. In some embodiments, the virus is SARS-COV-2.

### Systems for Treatment or Prevention of SARS-COV-2

In some embodiments, the systems provided herein encode antibodies or antigen binding fragments for the treatment and/or prevention of SARS-COV-2 infection and associated disease. In some embodiments, the antibodies or antigen binding fragments bind to a component of the SARS-COV-2 virus, such as a viral protein of the SARS-COV-2 virus.

In some embodiments, the antibodies or antigen binding fragments provided herein bind to one or more proteins expressed by the SARS-COV-2 virus. The antibody or antigen binding fragment may bind to any SARS-COV-2 protein. In preferred embodiments, the antibody or antigen binding fragment binds to a SARS-COV-2 protein involved in the infection of the SARS-COV-2 virus of a cell, thereby preventing infection of the cell, or binds to a SARS-CoV-2 protein expressed on the surface of an infected cell, thereby targeting the infected cell for killing by an immune cell (e.g., an NK cell).

In some embodiments, the SARS-COV-2 protein bound by the antibody or antigen binding fragment is a SARS-COV-2 spike protein or a SARS-COV-2 nucleocapsid protein. In some embodiments, the SARS-COV-2 protein is the SARS-COV-2 spike protein.

In some embodiments, the antibody or antigen binding fragment binds to the full-length SARS-COV-2 spike protein. In some embodiments, the antibody or antigen binding fragment bind specifically to a portion (e.g., a particular subunit) of the SARS-COV-2 spike protein. A schematic of the SARS-COV-2 spike protein domains is shown in FIG. 3A. In some embodiments, the portion of the SARS-COV-2 spike protein bound by the antibody or antigen binding fragment comprises one or more subunits of the SARS-COV-2 spike protein. In some embodiments, the subunits bound by the antibody or antigen binding fragment are selected from the N-terminal domain (NTD), the receptor binding domain (RBD), the S1 domain, the S2 domain, the fusion peptide domain, the heptad repeat domain 1 (HR1), the heptad repeat domain 2 (HR2), and the transmembrane domain (TM), or any combination thereof. In some embodiments, the portion of the SARS-COV-2 spike protein bound by the antibody or antigen binding fragment comprises the RBD.

In some embodiments, the SARS-COV-2 spike protein bound by the antibody or antigen binding fragment comprises one or more modifications to the sequence of SEQ ID NO: 200, which is the sequence of the originally identified Wuhan Spike protein sequence (MFVFLVLLPLVSSQCVNLTTRTQLPPAYTNSFTRGVYYPDKVFRSSVLHSTQDLFLP FFSNVTWFHAIHVSGTNGTKRFDNPVLPFNDGVYFASTEKSNIIRGWIFGTTLDSKTQ SLLIVNNATNVVIKVCEFQFCNDPFLGVYYHKNNKSWMESEFRVYSSANNCTFEYVS QPFLMDLEGKQGNFKNLREFVFKNIDGYFKIYSKHTPINLVRDLPQGFSALEPLVDLP IGINITRFQLLALHRSYLTPGDSSSGWTAGAAAYYVGYLQPRTFLLKYNENGTITDAV DCALDPLSETKCTLKSFTVEKGIYQTSNFRVQPTESIVRFPNITNLCPFGEVENATRFA SVYAWNRKRISNCVADYSVLYNSASFSTFKCYGVSPTKLNDLCFTNVYADSFVIRGD EVRQIAPGQTGKIADYNYKLPDDFTGCVIAWNSNNLDSKVGGNYNYLYRLFRKSNL KPFERDISTEIYQAGSTPCNGVEGFNCYFPLQSYGFQPTNGVGYQPYRVVVLSFELLH APATVCGPKKSTNLVKNKCVNFNFNGLTGTGVLTESNKKFLPFQQFGRDIADTTDA VRDPQTLEILDITPCSFGGVSVITPGTNTSNQVAVLYQDVNCTEVPVAIHADQLTPTW RVYSTGSNVFQTRAGCLIGAEHVNNSYECDIPIGAGICASYQTQTNSPRRARSVASQS IIAYTMSLGAENSVAYSNNSIAIPTNFTISVTTEILPVSMTKTSVDCTMYICGDSTECSN LLLQYGSFCTQLNRALTGIAVEQDKNTQEVFAQVKQIYKTPPIKDFGGFNFSQILPDP SKPSKRSFIEDLLFNKVTLADAGFIKQYGDCLGDIAARDLICAQKFNGLTVLPPLLTDE MIAQYTSALLAGTITSGWTFGAGAALQIPFAMQMAYRFNGIGVTQNVLYENQKLIA NQFNSAIGKIQDSLSSTASALGKLQDVVNQNAQALNTLVKQLSSNFGAISSVLNDILS RLDKVEAEVQIDRLITGRLQSLQTYVTQQLIRAAEIRASANLAATKMSECVLGQSKR VDFCGKGYHLMSFPQSAPHGVVFLHVTYVPAQEKNFTTAPAICHDGKAHFPREGVF VSNGTHWFVTQRNFYEPQIITTDNTFVSGNCDVVIGIVNNTVYDPLQPELDSFKEELD KYFKNHTSPDVDLGDISGINASVVNIQKEIDRLNEVAKNLNESLIDLQELGKYEQYIK WPWYIWLGFIAGLIAIVMVTIMLCCMTSCCSCLKGCCSCGSCCKFDEDDSEPVLKGV KLHYT). In some embodiments, the modifications of the SARS-COV-2 spike protein bound by the antibody or antigen binding fragment are modifications identified in a variant form of the SARS-COV-2 virus (e.g., the beta, gamma, delta, or omicron variants). In some embodiments, the modifications to the SARS-COV-2 spike protein bound by the antibody or antigen binding fragment are in the RBD of the variant form of the virus. Exemplary modifications of the SARS-COV-2 spike protein bound by the antibody or antigen binding fragment in the RBD of the selected variants can be found in Table 1 below. In some embodiments, additional modification to the spike protein outside of the RBD are bound by the antibody or antigen binding fragment. In some embodiments, the additional modifications bound by the antibody or antigen binding fragment are inside of the RBD and outside of the RBD.

TABLE 1
Select RBD mutations of select variants

| Variant | Lineage | Spike Receptor Binding Domain Mutations |
|---|---|---|
| Alpha | B.1.1.7 | N501Y. |
| Beta | B.1.351 | K417N, E484K, N501Y. |
| Gamma | P.1 | K417T, E484K, and N501Y. |
| Delta | B.1.617.2 | L452R, T478K. |
| Epsilon | B.1.427 | L452R. |
| | and | |
| | B.1.429 | |
| Zeta | P.2 | E484K. |
| Eta | B.1.525 | E484K. |
| Iota | B.1.526 | E484K. |
| Theta | P.3 | E484K; N501Y. |
| Kappa | B.1.617.1 | L452R, E484Q. |
| Lambda | C.37 | L452Q, F490S. |
| Omicron | BA.1 | G339D, S371L, S373P, S375F, K417N, N440K, G446S, |
| | | S477N, T478K, E484A, Q493R, G496S, Q498R, N501Y. |
| Omicron | BA.2 | G339D, S371F, S373P, S375F, T376A, D405N, R408S, |
| | | K417N, N440K, T478K, E484A, Q493R, Q498R, N501Y. |

In some embodiments, the SARS-COV-2 spike protein bound by the antibody or antigen binding fragment comprises one or more mutations found in the alpha, beta, gamma, delta, epsilon, zeta, eta, iota, theta, kappa, lambda, or omicron variants. In some embodiments, the SARS-COV-2 spike protein bound by the antibody or antigen binding fragment comprises one or more mutations found in the RBD of the alpha, beta, gamma, delta, epsilon, zeta, eta, iota, theta, kappa, lambda, or omicron variants. In some embodiments, the SARS-COV-2 spike protein bound by the antibody or antigen binding fragment comprises each of the mutations found in the RBD of the alpha, beta, gamma, delta, epsilon, zeta, eta, iota, theta, kappa, lambda, or omicron variants. In some embodiments, the SARS-COV-2 spike protein bound by the antibody or antigen binding fragment comprises each of the mutations found in the alpha, beta, gamma, delta, epsilon, zeta, eta, iota, theta, kappa, lambda, or omicron variants.

In some embodiments, the SARS-COV-2 spike protein bound by the antibody or antigen binding fragment comprises one or more mutations found in the beta, gamma, delta, or omicron variants. In some embodiments, the SARS-COV-2 spike protein bound by the antibody or antigen binding fragment comprises one or more mutations found in the RBD of the beta, gamma, delta, or omicron variants. In some embodiments, the SARS-COV-2 spike protein bound by the antibody or antigen binding fragment comprises each of the mutations found in the RBD of the beta, gamma, delta, or omicron variants. In some embodiments, the SARS-CoV-2 spike protein bound by the antibody or antigen binding fragment comprises each of the mutations found in the RBD of the beta variant. In some embodiments, the SARS-COV-2 spike protein bound by the antibody or antigen binding fragment comprises each of the mutations found in the RBD of the gamma variant. In some embodiments, the SARS-COV-2 spike protein bound by the antibody or antigen binding fragment comprises each of the mutations found in the RBD of the delta variant. In some embodiments, the SARS-COV-2 spike protein bound by the antibody or antigen binding fragment comprises each of the mutations found in the RBD of the omicron variant. In some embodiments, the SARS-COV-2 spike protein bound by the antibody or antigen binding fragment comprises 1, 2, 3, 4, 5, 6, 7, 8, 9, 10, 11, 12, or 13 of the mutations found in the RBD of the omicron variant.

In some embodiments, the SARS-COV-2 spike protein bound by the antibody or antigen binding fragment comprises a A67V, 69-70Del, T95I, 137-145Del, G142D, 143-145Del, Y145H, 211Del, L212I, ins214EPE, ins214TDR, A222V, G339D, R346K, R346S, V367F, S373P, S375F, P384L, N394S, Q414K, K417N, K417T, N439K, N440K, G446S, Y449H, Y449N, N450K, L452R, L452Q, S477N, T478K, V483A, E484A, E484K, E484Q, E484Del, F490R, F490S, Q493K, S494P, G496S, Q498R, N501T, N501Y, E516Q, T547K, Q613H, A653V, H655Y, G669S, Q677H, N679K, ins679GIAL, P681H, P681R, A701V, N764K, D796Y, N856K, Q954H, N969K, or L981F modification, or any combination thereof. In some embodiments, the SARS-COV-2 spike protein bound by the antibody or antigen binding fragment further comprises 1, 2, 3, 4, 6, 7, 8, 9, 10, 11, 12, 13, 14, 15, or more of A67V, 6970Del, T95I, 137-145Del, G142D, 143-145Del, Y145H, 211Del, L212I, ins214EPE, ins214TDR, A222V, G339D, R346K, R346S, V367F, S373P, S375F, P384L, N394S, Q414K, K417N, K417T, N439K, N440K, G446S, Y449H, Y449N, N450K, L452R, L452Q, S477N, T478K, V483A, E484A, E484K, E484Q, E484Del, F490R, F490S, Q493K, S494P, G496S, Q498R, N501T, N501Y, E516Q, T547K, Q613H, A653V, H655Y, G669S, Q677H, N679K, ins679GIAL, P681H, P681R, A701V, N764K, D796Y, N856K, Q954H, N969K, and L981F modifications.

In some embodiments, the SARS-COV-2 spike protein bound by the antibody or antigen binding fragment further comprises a A67V, 69-70Del, T95I, 137-145Del, G142D, 143145Del, Y145H, 211Del, L212I, ins214EPE, A222V, G339D, R346K, R346S, S371L, S373P, S375F, N394S, K417N, K417T, N440K, G446S, Y449H, Y449N, L452R, L452Q, S477N, T478K, E484A, E484K, E484Del, F490R, F490S, Q493K, G496S, Q498R, N501Y, T547K, Q613H, H655Y, Q677H, N679K, P681H, P681R, A701V, N764K, D796Y, N856K, Q954H, N969K, or L981F modification, or any combination thereof. In some embodiments, the SARS-COV-2 spike protein bound by the antibody or antigen binding fragment further comprises 1, 2, 3, 4, 5, 6, 7, 8, 9, 10, 11, 12, 13, 14, 15, or more of A67V, 69-70Del, T95I, 137-145Del, G142D, 143-145Del, Y145H, 211Del, L212I, ins214EPE, A222V, G339D, R346K, R346S, S371L, S373P, S375F, N394S, K417N, K417T, N440K, G446S, Y449H, Y449N, L452R, L452Q, S477N, T478K, E484A, E484K, E484Del, F490R, F490S, Q493K, G496S, Q498R, N501Y, T547K, Q613H, H655Y, Q677H, N679K, P681H, P681R, A701V, N764K, D796Y, N856K, Q954H, N969K, and L981F modifications.

In some embodiments, the SARS-COV-2 spike protein bound by the antibody or antigen binding fragment further comprises a A67V, 69-70Del, T95I, G142D, 143-145Del, Y145H, 211Del, L212I, ins214EPE, A222V, G339D, R346K, S371L, S373P, S375F, K417N, K417T, N440K, G446S, L452R, L452Q, S477N, T478K, E484A, E484K, F490S, Q493K, G496S, Q498R, N501Y, Y505H, T547K, H655Y, N679K, P681H, P681R, A701, N764K, D796Y, N856K, Q954H, N969K, or L981F modification, or any combination thereof. In some embodiments, the SARS-COV-2 spike protein bound by the antibody or antigen binding fragment further comprises 1, 2, 3, 4, 5, 6, 7, 8, 9, 10, 11, 12, 13, 14, 15, or more of A67V, 6970Del, T95I, G142D, 143-145Del, Y145H, 211Del, L212I, ins214EPE, A222V, G339D, R346K, S371L, S373P, S375F, K417N, K417T, N440K, G446S, L452R, L452Q, S477N, T478K, E484A, E484K, F490S, Q493K, G496S, Q498R, N501Y, Y505H, T547K, H655Y, N679K, P681H, P681R, A701, N764K, D796Y, N856K, Q954H, N969K, and L981F modifications.

In some embodiments, the SARS-COV-2 spike protein bound by the antibody or antigen binding fragment further comprises a A67V, 69-70Del, T95I, G142D, 143-145Del, 211Del, L212I, ins214EPE, G339D, S371L, S373P, S375F, K417N, K417T, N440K, G446S, S477N, L452R, T478K, E484A, E484K, Q493K, G496S, Q498R, N501Y, Y505H, T547K, H655Y, N679K, P681H, P681R, A701V, N764K, D796Y, N856K, Q954H, N969K, or L981F modifications, or any combination thereof. In some embodiments, the SARS-COV-2 spike protein bound by the antibody or antigen binding fragment further comprises 1, 2, 3, 4, 5, 6, 7, 8, 9, 10, 11, 12, 13, 14, 15, or more of A67V, 69-70Del, T95I, G142D, 143-145Del, 211Del, L212I, ins214EPE, G339D, S371L, S373P, S375F, K417N, K417T, N440K, G446S, S477N, L452R, T478K, E484A, E484K, Q493K, G496S, Q498R, N501Y, Y505H, T547K, H655Y, N679K, P681H, P681R, A701V, N764K, D796Y, N856K, Q954H, N969K, and L981F modifications. In some embodiments, the SARS-COV-2 spike protein bound by the antibody or antigen binding fragment at least partially aligns with the sequence set forth in SEQ ID NO: 200. In some embodiments, the SARS-COV-2 spike protein comprises an amino acid sequence having at least 90%, at least 95%, at least 96%, at least 97%, at least 98%, or at least 99% sequence identity with the sequence set forth in SEQ ID NO: 200. In some embodiments, the SARS-COV-2 spike protein bound by the antibody or antigen binding fragment comprises an amino acid sequence having at least 99%, at least 99.1%, at least 99.2%, at least 99.3%, at least 99.4%, at least 99.5%, at least 99.6%, at least 99.7%, at least 99.8%, or at least 99.9% sequence identity with the sequence set forth in SEQ ID NO: 200. In some embodiments, the SARS-CoV-2 spike protein bound by the antibody or antigen binding fragment comprises an amino acid sequence having at least 99.5% sequence identity with the sequence set forth in SEQ ID NO: 200. In some embodiments, the SARS-COV-2 spike protein bound by the antibody or antigen binding fragment comprises an amino acid sequence having at least 99.6% sequence identity with the sequence set forth in SEQ ID NO: 200. In some embodiments, the SARS-CoV-2 spike protein bound by the antibody or antigen binding fragment comprises an amino acid sequence having at least 99.7% sequence identity with the sequence set forth in SEQ ID NO: 200. In some embodiments, the SARS-COV-2 spike protein bound by the antibody or antigen binding fragment comprises an amino acid sequence having at least 99.8% sequence identity with the sequence set forth in SEQ ID NO: 200. In some embodiments, the SARS-CoV-2 spike protein bound by the antibody or antigen binding fragment comprises an amino acid sequence having at least 99.9% sequence identity with the sequence set forth in SEQ ID NO: 1.

In some embodiments, the anti-SARS-COV-2 antibodies bind to a plurality of variants of the SARS-COV-2 virus. In some embodiments, the anti-SARS-COV-2 antibodies bind to 2, 3, 4, 5, 6, or more variants of the Wuhan strain of SARS-COV-2 (SEQ ID NO: 200). In some embodiments, the anti-SARS-COV-2 antibody binds to 1, 2, 3, 4, 5, 6, or more variants of the Wuhan strain of SARS-COV-2 selected from alpha, beta, gamma, delta, epsilon, zeta, zeta, eta, iota, theta, kappa, lambda, and omicron. In some embodiments, the anti-SARS-COV-2 antibody binds to each of the beta, delta, gamma, and omicron variants. In some embodiments, the anti-SARS-COV-2 antibody binds to the RBD of each of the beta, delta, gamma, and omicron variants. In some embodiments, the anti-SARS-COV-2 antibody binds to the delta and omicron variants. In some embodiments, the anti-SARS-COV-2 antibody binds to the RBD of the delta and omicron variants.

In some embodiments, the anti-SARS-COV-2 antibody is an antibody or antigen binding fragment as provided herein.

In some embodiments, the SARS-COV-2 antibody or antigen binding fragment comprises a heavy chain variable region (HCVR) having an amino acid sequence of any one of SEQ ID NOS: 1, 9, 17, 25, 33, 41, 49, 57, 65, 73, 81, 89, or 97. In some embodiments, the anti-SARS-COV-2 antibody or antigen binding fragment heavy chain variable regions comprises a sequence with at least 70 percent (e.g., at least 80 percent, 85 percent, 90 percent, 91 percent, 92 percent, 93 percent, 94 percent, 95 percent, 96 percent, 97 percent, 98 percent, 99 percent, or greater) amino acid sequence identity with one of SEQ ID NOS: 1, 9, 17, 25, 33, 41, 49, 57, 65, 73, 81, 89, or 97.

In some embodiments, the anti-SARS-COV-2 antibody or antigen binding fragment comprises a heavy chain complementarity determining region 1 (HCDR1) having an amino acid sequence of one of SEQ ID NOS: 2, 10, 18, 26, 34, 42, 50, 58, 66, 74, 82, 90, or 98. In some embodiments, the anti-SARS-COV-2 antibody or antigen binding fragment HCDR1 comprises sequences with at least 70% (e.g., at least 80 percent, 85 percent, 90 percent, 91 percent, 92 percent, 93 percent, or 94 percent) amino acid sequence identity with one of SEQ ID NOS: 2, 10, 18, 26, 34, 42, 50, 57, 65, 73, 82, 90, or 98.

In some embodiments, the anti-SARS-COV-2 antibody or antigen binding fragment comprises a heavy chain complementarity determining region 2 (HCDR2) having an amino acid sequence of one of SEQ ID NOS: 3, 11, 19, 27, 35, 43, 51, 59, 67, 75, 83, 91, or 99. In some embodiments, the anti-SARS-COV-2 antibody or antigen binding fragment HCDR2 comprises sequences with at least 70% (e.g., at least 80 percent, 85 percent, 90 percent, 91 percent, 92 percent, 93 percent, or 94 percent) amino acid sequence identity with one of SEQ ID NOS: 3, 11, 19, 27, 35, 43, 51, 58, 66, 74, 83, 91, or 99.

In some embodiments, the anti-SARS-COV-2 antibody or antigen binding fragment comprises a heavy chain complementarity determining region 3 (HCDR3) having an amino acid sequence of one of SEQ ID NOS: 4, 12, 20, 28, 36, 44, 52, 60, 68, 76, 84, 92, or 100. In some embodiments, the anti-SARS-COV-2 antibody or antigen binding fragment HCDR3 comprises sequences with at least 70% (e.g., at least 80 percent, 85 percent, 90 percent, 91 percent, 92 percent, 93 percent, or 94 percent) amino acid sequence identity with one of SEQ ID NOS: 4, 12, 20, 28, 36, 44, 52, 60, 68, 76, 84, 92, or 100.

In some embodiments, the anti-SARS-COV-2 antibody or antigen binding fragment comprises a light chain variable region (LCVR) having an amino acid sequence of any one of SEQ ID NOS: 5, 13, 21, 29, 37, 45, 53, 61, 69, 77, 85, or 93. In some embodiments, the anti-SARS-COV-2 antibody or antigen binding fragment light chain comprises at least 70 percent (e.g., at least 80 percent, 85 percent, 90 percent, 91 percent, 92 percent, 93 percent, 94 percent, 95 percent, 96 percent, 97 percent, 98 percent, 99 percent, or greater) amino acid sequence identity to SEQ ID NOS: 5, 13, 21, 29, 37, 45, 53, 61, 69, 77, 85, or 93.

In some embodiments, the anti-SARS-COV-2 antibody or antigen binding fragment comprises a light chain complementarity determining region 1 (LCDR1) having an amino acid sequence of one of SEQ ID NOS: 6, 14, 22, 30, 38, 46, 54, 62, 70, 78, 86, or 94. In some embodiments, the anti-SARS-COV-2 antibody or antigen binding fragment LCDR1 comprises sequences with at least 70% (e.g., at least 80 percent, 85 percent, 90 percent, 91 percent, 92 percent, 93 percent, or 94 percent) amino acid sequence identity with one of SEQ ID NOS: 6, 14, 22, 30, 38, 46, 54, 62, 70, 78, 86, or 94.

In some embodiments, an anti-SARS-COV-2 antibody or antigen binding fragment comprises a light chain complementarity determining region 2 (LCDR2) having an amino acid sequence of one of SEQ ID NOS: 7, 15, 23, 31, 39, 47, 55, 63, 71, 79, 87, or 95. In some embodiments, the anti-SARS-COV-2 antibody or antigen binding fragment LCDR2 comprises sequences with at least 70% (e.g., at least 80 percent, 85 percent, 90 percent, 91 percent, 92 percent, 93 percent, or 94 percent) amino acid sequence identity with one of SEQ ID NOS: 7, 15, 23, 31, 39, 47, 55, 63, 71, 79, 87, or 95.

In some embodiments, an anti-SARS-COV-2 antibody or antigen binding fragment comprises a light chain complementarity determining region 3 (LCDR3) having an amino acid sequence of one of SEQ ID NOS: 8, 16, 24, 32, 40, 48, 56, 64, 72, 80, 88, or 96. In some embodiments, the anti-SARS-COV-2 antibody or antigen binding fragment LCDR2 comprises sequences with at least 70% (e.g., at least 80 percent, 85 percent, 90 percent, 91 percent, 92 percent, 93 percent, or 94 percent) amino acid sequence identity with one of SEQ ID NOS: 8, 16, 24, 32, 40, 48, 56, 64, 72, 80, 88, or 96.

In some embodiments, the anti-SARS-COV-2 antibody or antigen binding fragment comprises an HCDR1 according to SEQ ID NO: 2, an HCDR2 according to SEQ ID NO: 3, and HCDR3 according SEQ ID NO: 4. In some embodiments, the anti-SARS-COV-2 antibody or antigen binding fragment comprises an HCDR1 according to SEQ ID NO: 2, an HCDR2 according to SEQ ID NO: 3, and HCDR3 according SEQ ID NO: 4, and a VH having at least 80%, 85%, 90%, 95%, 96%, 97%, or 98% sequence identity with the sequence set forth in SEQ ID NO: 1. In some embodiments, the anti-SARS-COV-2 antibody or antigen binding fragment an LCDR1 according to SEQ ID NO: 6. an LCDR2 according to SEQ ID NO: 7, and an LCDR3 according to SEQ ID NO: 8. In some embodiments, the anti-SARS-COV-2 antibody or antigen binding fragment an LCDR1 according to SEQ ID NO: 6. an LCDR2 according to SEQ ID NO: 7, and an LCDR3 according to SEQ ID NO: 8, and a VL having at least 80%, 85%, 90%, 95%, 96%, 97%, or 98% sequence identity with the sequence set forth in SEQ ID NO: 5.

In some embodiments, the anti-SARS-COV-2 antibody or antigen binding fragment comprises an HCDR1 according to SEQ ID NO: 10, an HCDR2 according to SEQ ID NO: 11, and HCDR3 according SEQ ID NO: 12. In some embodiments, the anti-SARS-COV-2 antibody or antigen binding fragment comprises an HCDR1 according to SEQ ID NO: 10, an HCDR2 according to SEQ ID NO: 11, and HCDR3 according SEQ ID NO: 12, and a VH having at least 80%, 85%, 90%, 95%, 96%, 97%, or 98% sequence identity with the sequence set forth in SEQ ID NO: 9. In some embodiments, the anti-SARS-COV-2 antibody or antigen binding fragment an LCDR1 according to SEQ ID NO: 14. an LCDR2 according to SEQ ID NO: 15, and an LCDR3 according to SEQ ID NO: 16. In some embodiments, the anti-SARS-COV-2 antibody or antigen binding fragment an LCDR1 according to SEQ ID NO: 14. an LCDR2 according to SEQ ID NO: 15, and an LCDR3 according to SEQ ID NO: 16, and a VL having at least 80%, 85%, 90%, 95%, 96%, 97%, or 98% sequence identity with the sequence set forth in SEQ ID NO: 13.

In some embodiments, the anti-SARS-COV-2 antibody or antigen binding fragment comprises an HCDR1 according to SEQ ID NO: 18, an HCDR2 according to SEQ ID NO: 19, and HCDR3 according SEQ ID NO: 20. In some embodiments, the anti-SARS-COV-2 antibody or antigen binding fragment comprises an HCDR1 according to SEQ ID NO: 18, an HCDR2 according to SEQ ID NO: 19, and HCDR3 according SEQ ID NO: 20, and a VH having at least 80%, 85%, 90%, 95%, 96%, 97%, or 98% sequence identity with the sequence set forth in SEQ ID NO: 17. In some embodiments, the anti-SARS-COV-2 antibody or antigen binding fragment an LCDR1 according to SEQ ID NO: 22. an LCDR2 according to SEQ ID NO: 23, and an LCDR3 according to SEQ ID NO: 24. In some embodiments, the anti-SARS-COV-2 antibody or antigen binding fragment an LCDR1 according to SEQ ID NO: 22. an LCDR2 according to SEQ ID NO: 23, and an LCDR3 according to SEQ ID NO: 24, and a VL having at least 80%, 85%, 90%, 95%, 96%, 97%, or 98% sequence identity with the sequence set forth in SEQ ID NO: 21.

In some embodiments, the anti-SARS-COV-2 antibody or antigen binding fragment comprises an HCDR1 according to SEQ ID NO: 26, an HCDR2 according to SEQ ID NO: 27, and HCDR3 according SEQ ID NO: 28. In some embodiments, the anti-SARS-COV-2 antibody or antigen binding fragment comprises an HCDR1 according to SEQ ID NO: 26, an HCDR2 according to SEQ ID NO: 27, and HCDR3 according SEQ ID NO: 28, and a VH having at least 80%, 85%, 90%, 95%, 96%, 97%, or 98% sequence identity with the sequence set forth in SEQ ID NO: 25. In some embodiments, the anti-SARS-COV-2 antibody or antigen binding fragment an LCDR1 according to SEQ ID NO: 30. an LCDR2 according to SEQ ID NO: 31, and an LCDR3 according to SEQ ID NO: 32. In some embodiments, the anti-SARS-COV-2 antibody or antigen binding fragment an LCDR1 according to SEQ ID NO: 30. an LCDR2 according to SEQ ID NO: 31, and an LCDR3 according to SEQ ID NO: 32, and a VL having at least 80%, 85%, 90%, 95%, 96%, 97%, or 98% sequence identity with the sequence set forth in SEQ ID NO: 29.

In some embodiments, the anti-SARS-COV-2 antibody or antigen binding fragment comprises an HCDR1 according to SEQ ID NO: 34, an HCDR2 according to SEQ ID NO: 35, and HCDR3 according SEQ ID NO: 36. In some embodiments, the anti-SARS-COV-2 antibody or antigen binding fragment comprises an HCDR1 according to SEQ ID NO: 34, an HCDR2 according to SEQ ID NO: 35, and HCDR3 according SEQ ID NO: 36, and a VH having at least 80%, 85%, 90%, 95%, 96%, 97%, or 98% sequence identity with the sequence set forth in SEQ ID NO: 33. In some embodiments, the anti-SARS-COV-2 antibody or antigen binding fragment an LCDR1 according to SEQ ID NO: 38. an LCDR2 according to SEQ ID NO: 39, and an LCDR3 according to SEQ ID NO: 40. In some embodiments, the anti-SARS-COV-2 antibody or antigen binding fragment an LCDR1 according to SEQ ID NO: 38. an LCDR2 according to SEQ ID NO: 39, and an LCDR3 according to SEQ ID NO: 40, and a VL having at least 80%, 85%, 90%, 95%, 96%, 97%, or 98% sequence identity with the sequence set forth in SEQ ID NO: 37.

In some embodiments, the anti-SARS-COV-2 antibody or antigen binding fragment comprises an HCDR1 according to SEQ ID NO: 42, an HCDR2 according to SEQ ID NO: 43, and HCDR3 according SEQ ID NO: 44. In some embodiments, the anti-SARS-COV-2 antibody or antigen binding fragment comprises an HCDR1 according to SEQ ID NO: 42, an HCDR2 according to SEQ ID NO: 43, and HCDR3 according SEQ ID NO: 44, and a VH having at least 80%, 85%, 90%, 95%, 96%, 97%, or 98% sequence identity with the sequence set forth in SEQ ID NO: 41. In some embodiments, the anti-SARS-COV-2 antibody or antigen binding fragment an LCDR1 according to SEQ ID NO: 46. an LCDR2 according to SEQ ID NO: 47, and an LCDR3 according to SEQ ID NO: 48. In some embodiments, the anti-SARS-COV-2 antibody or antigen binding fragment an LCDR1 according to SEQ ID NO: 46. an LCDR2 according to SEQ ID NO: 47, and an LCDR3 according to SEQ ID NO: 48, and a VL having at least 80%, 85%, 90%, 95%, 96%, 97%, or 98% sequence identity with the sequence set forth in SEQ ID NO: 45.

In some embodiments, the anti-SARS-COV-2 antibody or antigen binding fragment comprises an HCDR1 according to SEQ ID NO: 50, an HCDR2 according to SEQ ID NO: 51, and HCDR3 according SEQ ID NO: 52. In some embodiments, the anti-SARS-COV-2 antibody or antigen binding fragment comprises an HCDR1 according to SEQ ID NO: 50, an HCDR2 according to SEQ ID NO: 51, and HCDR3 according SEQ ID NO: 52, and a VH having at least 80%, 85%, 90%, 95%, 96%, 97%, or 98% sequence identity with the sequence set forth in SEQ ID NO: 49. In some embodiments, the anti-SARS-COV-2 antibody or antigen binding fragment an LCDR1 according to SEQ ID NO: 54. an LCDR2 according to SEQ ID NO: 55, and an LCDR3 according to SEQ ID NO: 56. In some embodiments, the anti-SARS-COV-2 antibody or antigen binding fragment an LCDR1 according to SEQ ID NO: 54. an LCDR2 according to SEQ ID NO: 55, and an LCDR3 according to SEQ ID NO: 56, and a VL having at least 80%, 85%, 90%, 95%, 96%, 97%, or 98% sequence identity with the sequence set forth in SEQ ID NO: 53.

In some embodiments, the anti-SARS-COV-2 antibody or antigen binding fragment comprises an HCDR1 according to SEQ ID NO: 58, an HCDR2 according to SEQ ID NO: 59, and HCDR3 according SEQ ID NO: 60. In some embodiments, the anti-SARS-COV-2 antibody or antigen binding fragment comprises an HCDR1 according to SEQ ID NO: 58, an HCDR2 according to SEQ ID NO: 59, and HCDR3 according SEQ ID NO: 60, and a VH having at least 80%, 85%, 90%, 95%, 96%, 97%, or 98% sequence identity with the sequence set forth in SEQ ID NO: 57. In some embodiments, the anti-SARS-COV-2 antibody or antigen binding fragment an LCDR1 according to SEQ ID NO: 62. an LCDR2 according to SEQ ID NO: 63, and an LCDR3 according to SEQ ID NO: 64. In some embodiments, the anti-SARS-COV-2 antibody or antigen binding fragment an LCDR1 according to SEQ ID NO: 62. an LCDR2 according to SEQ ID NO: 63, and an LCDR3 according to SEQ ID NO: 64, and a VL having at least 80%, 85%, 90%, 95%, 96%, 97%, or 98% sequence identity with the sequence set forth in SEQ ID NO: 61.

In some embodiments, the anti-SARS-COV-2 antibody or antigen binding fragment comprises an HCDR1 according to SEQ ID NO: 66, an HCDR2 according to SEQ ID NO: 67, and HCDR3 according SEQ ID NO: 68. In some embodiments, the anti-SARS-COV-2 antibody or antigen binding fragment comprises an HCDR1 according to SEQ ID NO: 66, an HCDR2 according to SEQ ID NO: 67, and HCDR3 according SEQ ID NO: 68, and a VH having at least 80%, 85%, 90%, 95%, 96%, 97%, or 98% sequence identity with the sequence set forth in SEQ ID NO: 65. In some embodiments, the anti-SARS-COV-2 antibody or antigen binding fragment an LCDR1 according to SEQ ID NO: 70. an LCDR2 according to SEQ ID NO: 71, and an LCDR3 according to SEQ ID NO: 72. In some embodiments, the anti-SARS-COV-2 antibody or antigen binding fragment an LCDR1 according to SEQ ID NO: 70. an LCDR2 according to SEQ ID NO: 71, and an LCDR3 according to SEQ ID NO: 72, and a VL having at least 80%, 85%, 90%, 95%, 96%, 97%, or 98% sequence identity with the sequence set forth in SEQ ID NO: 69.

In some embodiments, the anti-SARS-COV-2 antibody or antigen binding fragment comprises an HCDR1 according to SEQ ID NO: 74, an HCDR2 according to SEQ ID NO: 75, and HCDR3 according SEQ ID NO: 76. In some embodiments, the anti-SARS-COV-2 antibody or antigen binding fragment comprises an HCDR1 according to SEQ ID NO: 74, an HCDR2 according to SEQ ID NO: 75, and HCDR3 according SEQ ID NO: 76, and a VH having at least 80%, 85%, 90%, 95%, 96%, 97%, or 98% sequence identity with the sequence set forth in SEQ ID NO: 73. In some embodiments, the anti-SARS-COV-2 antibody or antigen binding fragment an LCDR1 according to SEQ ID NO: 78. an LCDR2 according to SEQ ID NO: 79, and an LCDR3 according to SEQ ID NO: 80. In some embodiments, the anti-SARS-COV-2 antibody or antigen binding fragment an LCDR1 according to SEQ ID NO: 78. an LCDR2 according to SEQ ID NO: 79, and an LCDR3 according to SEQ ID NO: 80, and a VL having at least 80%, 85%, 90%, 95%, 96%, 97%, or 98% sequence identity with the sequence set forth in SEQ ID NO: 77.

In some embodiments, the anti-SARS-COV-2 antibody or antigen binding fragment comprises an HCDR1 according to SEQ ID NO: 82, an HCDR2 according to SEQ ID NO: 83, and HCDR3 according SEQ ID NO: 84. In some embodiments, the anti-SARS-COV-2 antibody or antigen binding fragment comprises an HCDR1 according to SEQ ID NO: 82, an HCDR2 according to SEQ ID NO: 83, and HCDR3 according SEQ ID NO: 84, and a VH having at least 80%, 85%, 90%, 95%, 96%, 97%, or 98% sequence identity with the sequence set forth in SEQ ID NO: 81. In some embodiments, the anti-SARS-COV-2 antibody or antigen binding fragment an LCDR1 according to SEQ ID NO: 86. an LCDR2 according to SEQ ID NO: 87, and an LCDR3 according to SEQ ID NO: 88. In some embodiments, the anti-SARS-COV-2 antibody or antigen binding fragment an LCDR1 according to SEQ ID NO: 86. an LCDR2 according to SEQ ID NO: 87, and an LCDR3 according to SEQ ID NO: 88, and a VL having at least 80%, 85%, 90%, 95%, 96%, 97%, or 98% sequence identity with the sequence set forth in SEQ ID NO: 85.

In some embodiments, the anti-SARS-COV-2 antibody or antigen binding fragment comprises an HCDR1 according to SEQ ID NO: 90, an HCDR2 according to SEQ ID NO: 91, and HCDR3 according SEQ ID NO: 92. In some embodiments, the anti-SARS-COV-2 antibody or antigen binding fragment comprises an HCDR1 according to SEQ ID NO: 90, an HCDR2 according to SEQ ID NO: 91, and HCDR3 according SEQ ID NO: 92, and a VH having at least 80%, 85%, 90%, 95%, 96%, 97%, or 98% sequence identity with the sequence set forth in SEQ ID NO: 89. In some embodiments, the anti-SARS-COV-2 antibody or antigen binding fragment an LCDR1 according to SEQ ID NO: 94. an LCDR2 according to SEQ ID NO: 95, and an LCDR3 according to SEQ ID NO: 96. In some embodiments, the anti-SARS-COV-2 antibody or antigen binding fragment an LCDR1 according to SEQ ID NO: 94. an LCDR2 according to SEQ ID NO: 95, and an LCDR3 according to SEQ ID NO: 96, and a VL having at least 80%, 85%, 90%, 95%, 96%, 97%, or 98% sequence identity with the sequence set forth in SEQ ID NO: 93.

In some embodiments, the anti-SARS-COV-2 antibody or antigen binding fragment comprises an HCDR1 according to SEQ ID NO: 98, an HCDR2 according to SEQ ID NO: 99, and HCDR3 according SEQ ID NO: 100. In some embodiments, the anti-SARS-COV-2 antibody or antigen binding fragment comprises an HCDR1 according to SEQ ID NO: 98, an HCDR2 according to SEQ ID NO: 99, and HCDR3 according SEQ ID NO: 100, and a VH having at least 80%, 85%, 90%, 95%, 96%, 97%, or 98% sequence identity with the sequence set forth in SEQ ID NO: 97.

In some embodiments, the therapeutic compositions provided herein provide for the expression of multiple antibodies or antigen binding fragments in the subject. In some embodiments, the therapeutic composition comprises a vector or multiple vectors which encode 2, 3, 4, 5, or more antibodies or antigen binding fragments which bind to a SARS-COV-2 protein. In some embodiments, each of the antibodies binds to the SARS-COV-2 spike protein. In some embodiments, each of the antibodies or antigen binding fragments thereof is an antibody or antigen binding fragment provided herein.

In some embodiments, the therapeutic compositions provided herein provide for the expression of multiple antibodies or antigen binding fragments in the subject. In some embodiments, the therapeutic composition comprises a vector or multiple vectors which encode 2, 3, 4, 5, or more antibodies or antigen binding fragments which bind to a SARS-COV-2 protein. In some embodiments, each of the antibodies binds to the SARS-COV-2 spike protein. In some embodiments, each of the antibodies or antigen binding fragments thereof is an antibody or antigen binding fragment provided herein. In some embodiments, the system provided herein includes two antibodies or antigen binding fragments thereof (e.g., Ab1 and Ab2). In some embodiments, each of the two antibodies binds the RBD of the SARS-COV-2 spike protein. In some embodiments, the two antibodies are capable of binding the RBD of the SARS-COV-2 spike protein at the same time. In some embodiments, the two antibodies bind to two separate epitopes of the SARS-COV-2 spike protein. An exemplary schematic of two antibodies binding in such a manner is shown in FIG. 3B. In FIG. 3B, two complementary antibodies bind the RBD at non-overlapping sites. One of the antibodies is a Class I anti-SARS-CoV-2 antibody which binds the RBD in an “up” orientation, wherein the second antibody is a Class IV, which binds RBD core region 1. Specifically, Ab1 falls into Class I and binds the “receptor-binding motif” (RBM) or ACE2 region of the spike RBD and is classified as an “ACE2 blocker”. Ab2 falls into class IV which does not overlap with the ACE2 binding site, but rather binds conserved region in the RBD (core I region).

In some embodiments, the antibodies or antigen binding fragments expressed comprise 2 or more antibodies which each comprise a VH and VL pair selected from SEQ ID NOs: 1 and 5, SEQ ID NOs: 9 and 13, SEQ ID NOs: 17 and 21, SEQ ID NOs: 25 and 29, SEQ ID NOs: 33 and 37, SEQ ID NOs: 41 and 45, SEQ ID NOs: 49 and 53, SEQ ID NOs: 57 and 61, SEQ ID NOs: 65 and 69, SEQ ID NOs: 73 and 77, SEQ ID NOs: 81 and 85, and SEQ ID NOs: 89 and 93.

### Microorganism Infections

In some embodiments, the vectors of the systems provided herein encode antibodies or antigen binding fragments which bind to an antigen associated with an infectious microorganism. In some embodiments, the system is effective to induce protection against infection by the microorganism. In some embodiments, the system is effective to mitigate, reduce, or eliminate infection of the microorganism. In some embodiments, the antigen associated with the microorganism a component of the microorganism. In some embodiments, the antigen associated with the microorganism is a protein, a glycan, a lipid membrane, a cell wall, or other component. In some embodiments, the antigen associated with the microorganism is a protein. In some embodiments, the microorganism is a bacterium. In some embodiments, the bacterium is a eukaryote. In some embodiments, the bacterium is prokaryotic. In some embodiments, the microorganism is a fungus.

In some embodiments, the microorganism for which the antibody or antigen binding fragment is targeted is Bacillus anthracis, Corynebacterium diphtheria, Bordetella pertussis, Streptococcus pneumonia, Haemophilus influenza, Salmonella typhimurium, a Shigella species, a Streptococcus species, Chlamydia trachomatis, Yersinia pestis, Methicillin-resistant Staphylococcus aureus (MRSA), Staphylococcus aureus, Clostridium tetani, Vibrio cholera, Escherichia coli, Klebsiella pneumonia, Borrelia burgdorferi, Borrelia mayonii, Clostridioides difficile, Pseudomonas aeruginosa, Helicobacter pylori, Streptococcus pyogenes, Francisella tularensis, an Acinetobacter species, Neisseria gonorrhoeae, a Leptospira species, Coxiella burnetii, Clostridium botulinum, Burkholderia pseudomallei, a gram-negative bacteria, Salmonella paratyphi, Mycobacterium leprae, a Brucella species, a Campylobacter species, Listeria monocytogenes, Mycobacterium avium, Mycoplasma pneumonia, a Rickettsia species, a Anaplasma species, an Ehrlichia species, a Neorickettsia species, a Neoehrlichia species, a Orientia species, Mycobacterium tuberculosis, Anaplasam phagocytophilum, Orientia tsutsugamushi, or a Bartonella species.

### Parasitic Infections

In some embodiments, the vectors of the systems provided herein encode antibodies or antigen binding fragments which bind to an antigen associated with an infectious parasite. In some embodiments, the system is effective to induce protection against infection by the parasite. In some embodiments, the system is effective to mitigate, reduce, or eliminate infection of the parasite. In some embodiments, the antigen associated with the parasite a component of the parasite. In some embodiments, the antigen associated with the parasite is a protein, a glycan, a lipid membrane, a cell wall, or other component.

In some embodiments, the parasite is the parasite is a Babesia species, Ancylostoma duodenale, Necator americanus, Sarcoptes scabiei, Ascaris lumbricoides, Schistosoma mansoni, Taenia solium, Enterobius vermicularis, Wuchereria bancrofti, Toxoplasma gondii, Giardia lamblia, Entamoeba histolytica, a Plasmodium species, a Leishmania species, Trypanosoma cruzi, a Schistosoma species, a Cryptosporidium species, Trypanosoma brucei, Wuchereria bancrofti, Brugia malayi, Brugia timori, Entamoeba histolytica, or Onchocerca volvulus.

### Immune Checkpoints for Infectious Diseases

In some embodiments, the vectors of the systems provided herein encode antibodies or antigen binding fragments which bind to an immune checkpoint molecule. In some embodiments, such immune checkpoint binders act as inhibitors of the immune checkpoint. In some embodiments, the immune checkpoint binder is useful in the treatment of an infectious disease. In some embodiments, the immune checkpoint molecule is PD-1, PD-L1, CTLA-4, TIM-3, TIGIT, 4-1BB (CD137), GITR (CD357), or a killer IgG-like receptor (KIR). In some embodiments, the immune checkpoint molecule is PD-1. In some embodiments, the immune checkpoint molecule is PD-L1.

### Other Diseases and Indications

Other indications or diseases which can be treated and/or prevented using the systems provided herein include any indication or disease which can be treated with an antibody. Nonlimiting examples of such disease or indications include cancer, autoimmune disease, an inflammatory disease, an autoinflammatory disease, acute toxicity from an environmental factor (e.g., a toxin such as an environmental toxin or other toxin, such as snake venom, etc.), or allergies.

In some embodiments, the antibody or antigen binding fragment of a system as provided herein is specific for a cancer antigen. In some embodiments, the cancer antigen is selected from the group consisting of programmed cell death 1 (PD1) programmed cell death ligand 1 (PDL1), CD5, CD20, CD19, CD22, CD30, CD33, CD40, CD44, CD52, CD74, CD103, CD137, CD123, CD152, a carcinoembryonic antigen (CEA), an integrin, an epidermal growth factor (EGF) receptor family member, a vascular epidermal growth factor (VEGF), a proteoglycan, a disialoganglioside, B7-H3, cancer antigen 125 (CA-125), epithelial cell adhesion molecule (EpCAM), vascular endothelial growth factor receptor 1, vascular endothelial growth factor receptor 2, a tumor associated glycoprotein, mucin 1 (MUC1), a tumor necrosis factor receptor, an insulin-like growth factor receptor, folate receptor a, transmembrane glycoprotein NMB, a C-C chemokine receptor, prostate specific membrane antigen (PSMA), recepteur d'origine nantais (RON) receptor, cytotoxic T-lymphocyte antigen 4 (CTLA4), Colon cancer antigen 19.9, gastric cancer mucin antigen 4.2, colorectal carcinoma antigen A33, ADAM-9, AFP oncofetal antigen-alpha-fetoprotein, ALCAM, BAGE, beta-catenin, Carboxypeptidase M, B1, CD23, CD25, CD27, CD28, CD36, CD45, CD46, CD52, CD56, CD79a/CD79b, CD317, CDK4, CO-43 (blood group Leb), CO-514 (blood group Leª), CTLA-1, Cytokeratin 8, DR5, E1 series (blood group B), Ephrin receptor A2 (EphA2), Erb (ErbB1, ErbB3, ErbB4), lung adenocarcinoma antigen F3, antigen FC10.2, GAGE-1, GAGE-2, GD2/GD3/GD49/GM2/GM3, GICA 19-9, gp37, gp75, gp100, HER-2/neu, human milk fat globule antigen, human papillomavirus-E6/human papillomavirus-E7, high molecular weight melanoma antigen (HMW-MAA), differentiation antigen (I antigen), I (Ma) as found in gastric adenocarcinomas, Integrin Alpha-V-Beta-6, Integrinβ6 (ITGβ6), Interleukin-13 Receptor a2 (IL13Ra2), JAM-3, KID3, KID31, KS 1/4 pan-carcinoma antigen, KSA (17-1A), human lung carcinoma antigen L6, human lung carcinoma antigen L20, LEA, LUCA-2, M1:22:25:8, M18, M39, MAGE-1, MAGE-3, MART, Myl, MUM-1, N-acetylglucosaminyltransferase, neoglycoprotein, NS-10, OFA-1 and OFA-2, Oncostatin M (Oncostatin Receptor Beta), rho15, prostate specific antigen (PSA), PSMA, polymorphic epithelial mucin antigen (PEMA), PIPA, prostatic acid phosphate, R24, ROR1, SSEA-1, SSEA-3, SSEA-4, sTn, T cell receptor derived peptide, T5A7, Tissue Antigen 37, TAG-72, TL5 (blood group A), a TNF-α receptor (TNFαR), TNFαR, TNFγR, TRA-1-85 (blood group H), Transferrin Receptor, TSTA tumor-specific transplantation antigen, VEGF-R, Y hapten, Ley, and 5T4.

In some embodiments, the antibody or antigen binding fragment of a system as provided herein is specific for an allergen. In some embodiments, the allergen is derived from a mite, an insect, a pollen, an animal epithelium, a mold, meat, a fish, a crustacean, a fruit, a nut, a vegetable, a flour or bran, a milk, an egg, a spice, hay, silk, cotton, latex, a yeast, a grass, a tree, a cereal, or an animal hair. In some embodiments, the mite allergen is Der p 1, Der f 1, or Blomia tropicalis. In some embodiments, the insect allergen is derived from cockroach or locust. In some embodiments, the pollen allergen is derived from mugwort, birch, nettle, chrysanthemum, alder, spruce, Lamb's Quarters, goldenrod, Humulus japonicus, pine, orchard grass, dandelion, corn, poplar, plane tree, short ragweed, elm, English Plantain, willow tree, wheat, Timothy grass, queen palm, mulberry, rape, or ryegrass. In some embodiments, the animal epithelia allergen is derived from dog epithelia, cat epithelia, goat epithelia, duck feather, or feather. In some embodiments, the mold allergen is derived from Alternaria tenuis, Botrytis c., Candida albicans, Cladosporium h., Curvularia l., Penicillium notatum, Pullalaria pullulans, Trichophyton mentagrophytes, Fusarium globosum, Helminthosporium halodes, Aspergillus f., Mucor mucedo, Rhizopus nigricans, or Serpula lacrymans. In some embodiments, the meat allergen is derived from mutton, chicken, beef, pork, duck, turkey, or goose. In some embodiments, the fish or crustacean allergen is derived from cod, carp, catfish, tuna, scallop, crab meat, shrimp, spiny lobster, or mussel. In some embodiments, the fruit or nut allergen is derived from pineapple, apple, orange, banana, mango, strawberry, peanut, cashew nut, tangerine, paprika, peach, pear, tomato, walnut, grape, sunflower seed, almond, hazelnut, pistachio, pine nut, cocoa bean, chestnut, Macadamia nut, brazil nut, lupins bean, pecan nut, or pumpkin seed. In some embodiments, the vegetable allergen is derived from potato, parsley, spinach, soybean, spring onion, leek, or cabbage. In some embodiments, the flour or bran allergen is derived from rice, corn flour, wheat flour, buck wheat, or green bean. In some embodiments, the milk or egg allergen is derived from cow's milk, whole egg, egg white, or egg yolk. In some embodiments, the spice allergen is derived from cocoa, cinnamon, paprika, black pepper, sesame, or garlic. In some embodiments, the allergen is derived from hay, silk, cotton, latex, or yeast (e.g., Baker's Yeast). In some embodiments, the grass allergen is derived from Velvet Grass, Orchard Grass, Ryegrass, Timothy Grass, Kentucky Bluegrass, or Meadow Fescue. In some embodiments, the tree allergen is derived from alder, hazel, poplar, elm, willow, birch, oak, or platanus. In some embodiments, the grass allergen is derived from mugwort, nettle, dandelion, or English plantain. In some embodiments, the cereal allergen is derived from grass, barley, oat, rye, or wheat. In some embodiments, the grass allergen is derived from an Australian grass. In some embodiments, the grass allergen is derived from Bahia grass, Johnson grass, Burmuda grass, Velvet grass, or Canary grass. In some embodiments, the animal hair allergen is derived from hamster, dog, rabbit, cat, or guinea pig.

In some embodiments, the antibody or antigen binding fragment thereof binds specifically to an antigen implicated in an inflammatory disease. In some embodiments, the inflammatory disease is an allergy, asthma, coeliac disease, glomerulonephritis, hepatitis, or inflammatory bowel disease. In some embodiments, the inflammatory disease is Mast Cell Activation Syndrome (MCAS).

In some embodiments, the antibody or antigen binding fragment thereof binds specifically to an antigen implicated in an autoinflammatory disease. In some embodiments, the inflammatory disease is an autoinflammatory disease selected from Familial Mediterranean fever (FMF), Cryopyrin-associated periodic syndromes (CAPS), TNF receptor-associated periodic syndrome (TRAPS), Deficiency of IL-1-receptor antagonist (DIRA), or Hyper IgD syndrome (HIDS).

In some embodiments, the antibody or antigen binding fragment thereof binds specifically to an antigen implicated in an autoimmune disease. In some embodiments, the autoimmune disease is an autoimmune disease selected from rheumatoid arthritis, psoriasis, Guillain-Barre syndrome, Graves' disease, Mysathenia gravis, vasculitis, lupus, Type 1 diabetes, Hashimoto's disease, inflammatory bowel disease, Celiac disease, or multiple sclerosis (MS).

### Lipid Vesicles

In certain aspects the antibody or antigen binding fragment expression systems provided herein comprise lipid vesicles. A lipid vesicle comprises one or more lipid components which can encapsulate a vector provided herein (e.g., a DNA plasmid). In some embodiments, the lipid vesicles comprise one or more protein components contacting or disposed at least partially within the lipid. In some embodiments, the lipid vesicle includes lipid nanoparticle (LNP) compositions and compositions wherein an LNP encapsulates a polynucleotide construct (e.g., a vector as provided herein, such as plasmid DNA) comprising a coding region for an antibody or antigen binding fragment as provided herein.

In some embodiments, compositions comprising a plasmid DNA encapsulated with a LNP or other lipid vesicle formulation are non-toxic and non-immunogenic in animals at doses of >15 mg/kg and exhibit an efficiency in excess of 80× greater than that achievable with neutral lipid compositions and 2-5× greater than that achievable with cationic lipid compositions. In some embodiments, LNP or other lipid vesicle cargo is deposited directly into the cytoplasm, thereby bypassing the endocytic pathway.

Within further embodiments, the present disclosure lipid vesicles for the targeted production of an antibody or antigen binding fragment within a target cell (which is then preferably excreted from the cell), which lipid vesicle composition comprises: (a) a lipid nanoparticle vector for the non-specific delivery of a nucleic acid to mammalian cells, wherein the lipid nanoparticle includes one or more lipid(s) and one or more fusogenic membrane protein(s), and (b) an expression: construct for the preferential production of an antibody or antigen binding fragment within a target cell.

Lipid vesicle compositions according to certain aspects of these embodiments include one or more lipid(s) at a concentration ranging from 1 mM to 100 mM, or from 5 mM to 50 mM, or from 10 mM to 30 mM, or from 15 mM to 25 mM. Lipid vesicle formulations exemplified herein can include one or more lipid(s) at a concentration of about 20 mM.

Within certain illustrative lipid vesicle compositions, one or more lipid(s) is selected from 1,2-dioleoyl-3-dimethylammonitim-propane (DODAP), 1,2-dioleoyl-3trimethylammonium-propane (DOTAP), 1,2-dioleoyl-sn-glycero-3-phosphoethanolamine (DOPE), Cholesterol, and 1,2-dimyristoyl-rac-glycero-3-methoxypolyethylene glycol (DMG-PEG). LNP compositions may contain two or more lipids selected from the group consisting of DODAP, DOTAP, DOPE, Cholesterol, and DMG-PEG.

Exemplified herein are lipid compositions including DODAP, DOTAP, DOPE, Cholesterol, and DMG-PEG at a molar ratio of 35-55 mole % DODAP:10-20 mole % DOTAP:22.5-37.5 mole % DOPE:4-8 mole % Cholesterol:3-5 mole % DMG-PEG; or at a molar ratio of about 45 mole % DODAP:about 15: mole % DOTAP about 30 mole % DOPE:about 6 mole % Cholesterol about 4 mole % DMG-PEG. Within certain aspects, the lipid vesicle compositions include DODAP, DOTAP, DOPE, Cholesterol, and DMG-PEG at a molar ratio of 45 mole % DODAPil 5 mole % DOTAP: 30 mole % DOPE:6 mole % Cholesterol:4 mole % DMG-PEG.

Lipid vesicle formulations according to other aspects of these embodiments include one or more fusogenic membrane protein(s) at a concentration ranging from 0.5 M to 20 M, or from 1 M to 10 M, or from 3 M to 4 M. Exemplified herein are lipid vesicle formulations wherein fusogenic membrane protein(s) are present at a concentration of about 3.5 M, about 5 M, about 7.5 M, about 10 M, about 12.5 M, about 15 M, about 20 M. Exemplary, suitable fusogenic membrane protein(s) include those provided herein, including a p15× fusogenic membrane protein (SEQ ID NO: 201), a pl4 fusogenic membrane protein (SEQ ID NO: 202), and a p14e15 fusogenic membrane protein (SEQ ID NO: 203).

Within additional aspects of these embodiments, lipid vesicle formulations include vectors comprising polynucleotide sequences encoding one or more antibody or antigen binding fragment as set forth above.

In some embodiments, the pharmaceutical compositions provided herein comprise proteolipid vehicles (PLV). In some embodiments, the proteolipid vehicle encapsulates one or more other parts of the pharmaceutical composition (e.g., the DNA vector, such as any DNA vector provided herein).

Exemplified herein are lipid vesicle formulations including vectors (e.g., DNA plasmids) at a concentration ranging from 20 g/mL to 1.5 mg/mL, of from 100 g/mL to 500 g/mL, or at a concentration of about 250 g/mL.

A suitable exemplary lipid vesicle formulation includes the following: for each 1 mL of lipid vesicle, the lipid concentration is about 20 mM, the DNA content is about 250 g, and the fusogenic protein (e.g, p14 or p14e15) is at about 3.5 M wherein the lipid formulation comprises DODAP: DOTAP: DOPE: Cholesterol: DMG-PEG at a mole % ratio of about 45:15:30:6:4, respectively.

The lipid vesicle comprises one or more lipid components. In some embodiments, the lipids of the lipid vesicle are non-immunogenic lipids. In some embodiments, the lipids of the lipid vesicle comprise naturally occurring lipids. In some embodiments, the lipids of the lipid vesicle comprise naturally occurring mammalian lipids. In some embodiments, the lipids of the lipid vesicle comprise naturally occurring human lipids.

In some embodiments, the lipid vesicle comprises a minimal amount of cationic lipid. cationic lipids are used in certain lipid vesicle formulations in order to facilitate the fusion of the lipid vesicle with another desired membrane. However, in some embodiments, proteolipid vesicles provided herein use alternative strategies for the fusion of the lipid vesicle with a desired cell membrane (e.g., a fusogenic membrane protein). Thus, the lipid vesicles provided herein in some instances use less cationic lipids than other preparations, which makes the lipid vesicles provided herein less toxic. Due to their positive charge, cationic lipids have been employed for condensing negatively charged DNA molecules and to facilitate the encapsulation of DNA into liposomes. In some embodiments, the lipid vesicle comprises less than 10%, less than 5%, less than 4%, less than 3%, less than 2%, less than 1%, less than 0.5%, less than 0.4%, less than 0.3%, less than 0.2%, or less than 0.1% cationic lipid content in the proteolipid vehicle (w/w of total lipid content). In some embodiments, the lipid vesicle comprises a molar ratio of ionizable lipid to vector (e.g., plasmid) which is less than 100:1, less than 75:1, less than 50:1, less than 40:1, less than 30:1, less than 25:1, or less than 20:1. In some embodiments, the molar ratio of ionizable lipid to vector (e.g., plasmid) is between 2.5:1 and 20:1.

### Fusogenic Membrane Proteins

In some embodiments, the proteolipid vehicles comprise a fusogenic membrane protein. A fusogenic membrane protein is membrane bound or associated protein which facilitates lipid to lipid membrane fusion of two separate lipid membranes. Many such fusogenic membrane proteins are known in the art.

In some embodiments, the fusogenic membrane protein is derived from a virus. Examples of such virus derived fusogenic membrane proteins include influenza virus hemagglutinin (HA) proteins, Sendai virus F proteins, Filoviridae family ebolavirus glycoproteins, Retroviridae family glycoprotein 41, Togaviridae family alphaviruse envelope protein El, Flaviviridae family Flavivirus envelope protein, Herpesviridae family Herpesvirus glycoprotein B, Rhabdoviridae family SVS G proteins, Reoviridae family fusion-associated small transmembrane proteins (FAST), and derivatives thereof.

Within other aspects of these embodiments, lipid vesicles are fusogenic lipid vesicles, such as fusogenic lipid vesicles comprising a fusogenic membrane protein, such as a fusogenic p14 FAST membrane fusion protein from reptilian reovirus to catalyze lipid mixing between the lipid vesicle and target cell plasma membrane. Suitable fusogenic membrane proteins are described in PCT Patent Publication Nos. WO2012/040825A1 and WO2002/044206A2, Lau, Biophys. J. g£: 272 (2004), Nesbitt, Master of Science Thesis (2012), Zijlstra, AACR (2017), Mrlouah, PAACRAM 77/13Supnn: Abst 5143 (2017), Krabbe, Cancers 10:216 (2018), Sanchez-Garria, ChemComm 53:4565 (2017), Clancy,/Virology 83/71:2941 (2009), Sudo, J Control Release 255:1 (2017), Wong, Cancer Gene Therapy 23:355 (2016), and Corcoran, JBC 281/421:31778 (2006), each of which is incorporated by reference as if set forth herein in its entirety. Further examples FAST membrane proteins, fusion proteins thereof, and exemplary formulations can be found in PCT Publication No. WO2022/067446A1, which is hereby incorporated by reference as if set forth herein in its entirety.

Examples of fusogenic FAST proteins include the p15 and p14e15 proteins having the amino acid sequences presented in Table 2. In some embodiments, the FAST protein of a lipid vesicle provided herein comprises a sequence having at least 80%, at least 85%, at least 90%, at least 95%, at least 98%, or 100% sequence identity with the sequence of p15× set forth below. In some embodiments, the FAST protein of a lipid vesicle provided herein comprises a sequence having at least 80%, at least 85%, at least 90%, at least 95%, at least 98%, or 100% sequence identity with the sequence of p14 set forth below. In some embodiments, the FAST protein of a lipid vesicle provided herein comprises a sequence having at least 80%, at least 85%, at least 90%, at least 95%, at least 98%, or 100% sequence identity with the sequence of p14e15 set forth below.

TABLE 2

| | | |
|---|---|---|
| p15x | MGSGPSNFVNHAPGEAIVTGLEKG | SEQ ID NO: |
| | ADKVAGTISHTIFVEIVSSSTGIIIAV | 201 |
| | GIFAFIFSFLYKLLQWYNRKSKNK | |
| | KRKEQIREQIELGLLSYGAGVASLP | |
| | LLNVIAHNPGSVISATPIYKGPCTG | |
| | VPNSRLLQITSGTAEENTRILNHDG | |
| | RNPDGSINV | |
| p14 | MGSGPSNFVNHAPGEAIVTGLEKG | SEQ ID NO: |
| | ADKVAGTISHTIWEVIAGLVALLT | 202 |
| | FLAFGFWLFKYLQKRRERRRQLTE | |
| | FQKRYLRNSYRLSEIQRPISQHEYE | |
| | DPYEPPSRRKPPPPPYSTYVNIDNV | |
| | SAI | |
| | MGSGPSNFVNHAPGEAIVTGLEK | |
| | G | |
| p14e15 | ADKVAGTISHTIWEVIAGLVALLT | SEQ ID NO: |
| | FLAFGFWLFKYLQWYNRKSKNKK | 203 |
| | RKEQIREQIELGLLSYGAGVASLPL | |
| | LNVIAHNPGSVISATPIYKGPCTGV | |
| | PNSRLLQITSGTAEENTRILNHDGR | |
| | NPDGSINV | |

Preferred fusogenic membrane proteins are those which are non-immunogenic (e.g., do not produce an immune response specific to the fusogenic membrane protein upon administration to a subject). In some cases, such fusogenic membrane proteins allow for repeated administration of the pharmaceutical compositions provided herein and/or for enhanced delivery of enclosed material (e.g., DNA vectors as provided herein) to target cells.

In some embodiments, the fusogenic membrane protein is a FAST protein. Specific examples of FAST proteins are described in U.S. Pat. No. 8,252,901 and U.S. Pat. App. No. 2019/0367566, each of which is incorporated by reference as if set forth herein in its entirety.

FAST proteins are a unique family of fusogenic membrane proteins encoded by fusogenic reoviruses. FAST proteins include: p10, p14, p15 and p22. At 95 to 198 amino acids in size, the FAST proteins are the smallest known viral membrane fusion proteins. Rather than mediating virus-cell fusion, the FAST proteins are non-structural viral proteins that are expressed on the surfaces of virus-infected or -transfected cells, where they induce cell-cell fusion and the formation of multinucleated syncytia. A purified FAST protein, when reconstituted into liposome membranes, induces liposome-cell and liposome-liposome fusion, indicating the FAST proteins are bona fide membrane fusion proteins.

In contrast to most enveloped viral fusion proteins in which the cytoplasmic tail is extremely short relative to the overall size of the protein, the FAST proteins all have an unusual topology that partitions the majority of the protein to the membrane and cytoplasm, exposing ectodomains of just 20 to 43 residues to the extracellular milieu. Despite the diminutive size of their ectodomains, both p14 and p10 encode patches of hydrophobicity (HP) hypothesized to induce lipid mixing analogously to the fusion peptides encoded by enveloped viral fusion proteins. The p14 HP is comprised of the N-terminal 21 residues of the protein, but peptides corresponding to this sequence require the inclusion of the N-terminal myristate moiety to mediate lipid mixing. Nuclear magnetic resonance (NMR) spectroscopy revealed that two proline residues within the p14 HP form a protruding loop structure presenting valine and phenylalanine residues at the apex and connected to the rest of the protein by a flexible linker region. The p10 HP on the other hand, flanked by two cysteine residues that form an intramolecular disulfide bond, may have more in common with the internal fusion peptides of the Ebola virus and avian leukosis and sarcoma virus (ALSV) glycoproteins, and likely adopts a cystine-noose structure that forces solvent exposure of conserved valine and phenylalanine residues for membrane interactions. In contrast to p14 and p10, the 20 residue ectodomain of p15 completely lacks a hydrophobic sequence that could function as a traditional fusion peptide. In the absence of such a motif, the p15 ectodomain instead encodes a polyproline helix that has been proposed to function as a membrane destabilizing motif.

FAST proteins with improved properties for facilitating membrane fusion in the context of synthetic lipid vesicles (e.g., the proteolipid vehicles of the instant disclosure) have been previously described (e.g., U.S. Pat. No. 10,227,386).

In some embodiments, the FAST protein comprises domains from one or more FAST proteins selected from p10, p14, p15, and p22. In some embodiments, the FAST protein comprises an ectodomain, a transmembrane domain, and an endodomain.

In some embodiments, the FAST protein comprises an endodomain having at least about 80%, at least about 85%, at least about 90%, or at least about 95% sequence identity with an endodomain from p10, p14, p15, or p22. In some embodiments, the FAST protein comprises an endodomain from p10, p14, p15, or p22. In some embodiments, the FAST protein comprises an endodomain having at least about 80%, at least about 85%, at least about 90%, or at least about 95% sequence identity with an endodomain from p15. In some embodiments, the FAST protein comprises an endodomain from p15.

In some embodiments, the FAST protein comprises a transmembrane domain from a wildtype FAST protein, or a derivative thereof. In some embodiments, the FAST protein comprises a transmembrane domain having at least about 80%, at least about 85%, at least about 90%, or at least about 95% sequence identity with a transmembrane domain from p10, p14, p15, or p22. In some embodiments, the transmembrane domain comprises 23 amino acid residues, at least two hydrophobic, β-branched residues adjacent the ectodomain, three consecutive serine residues immediately adjacent the at least two hydrophobic, β-branched residues, and a glycine residue at positions 7 and 13 from the junction between the ectodomain and the first hydrophobic, β-branched residue. In some embodiments, the transmembrane domain comprises an amino acid sequence having at least about 80%, at least about 85%, at least about 90%, or at least about 95% sequence identity with the sequence of IVSSSTGIIIAVGIFAFIFSFLY (SEQ ID NO: 204).

In some embodiments, the FAST protein comprises an ectodomain from a wildtype FAST protein, or a derivative thereof. In some embodiments, the FAST protein comprises an ectodomain having at least about 80%, at least about 85%, at least about 90%, or at least about 95% sequence identity with an ectodomain from p10, p14, p15, or p22. In some embodiments, the FAST protein comprises an ectodomain from p10, p14, p15, or p22. In some embodiments, the FAST protein comprises an endodomain having at least about 80%, at least about 85%, at least about 90%, or at least about 95% sequence identity with an ectodomain from p14. In some embodiments, the FAST protein comprises an ectodomain from p14.

In some embodiments, a FAST protein as provided herein comprises an ectodomain comprising a sequence with at least 80%, at least 85%, at least 90%, at least 95%, at least 98%, or 100% sequence identity that of a p14 FAST protein (e.g., the sequence defined by the sequence MGSGPSNFVNHAPGEAIVTGLEKGADKVAGTISHTIWE (SEQ ID NO: 205)) and comprising a functional myristoylation motif; a transmembrane domain comprising 23 amino acid residues, at least two hydrophobic, β-branched residues adjacent the ectodomain, three consecutive serine residues immediately adjacent the at least two hydrophobic, β-branched residues, and a glycine residue at positions 7 and 13 from the junction between the ectodomain and the first hydrophobic, β-branched residue; and an endodomain comprising a sequence with at least 80%, at least 85%, at least 90%, at least 95%, at least 98%, or 100% sequence identity with the sequence a p15 endodomain (e.g., as sequence defined by KLLQWYNRKSKNKKRKEQIREQIELGLLSYGAGVASLPLLNVIAHNPGS (SEQ ID NO: 206) or VISATPIYKGPCTGVPNSRLLQITSGTAEENTRILNHDGRNPDGSINV (SEQ ID NO: 207)).

In some embodiments, the FAST protein comprises an amino acid having at least about at least about 80%, at least about 85%, at least about 90%, at least about 95%, at least about 96%, at least about 97%, at least about 98%, at least about 99%, or 100% sequence identity with the sequence of MGSGPSNFVNHAPGEAIVTGLEKGADKVAGTISHTIFVEIVSSSTGIIIAVGIFAFIFSFL YKLLQWYNRKSKNKKRKEQIREQIELGLLSYGAGVASLPLLNVIAHNPGSVISATPIY KGPCTGVPNSRLLQITSGTAEENTRILNHDGRNPDGSINV (SEQ ID NO: 203).

In some embodiments, the FAST protein is provided from a commercial vendor. In some embodiments, the FAST protein is part of the Fusogenix platform prepared by Entos Pharmaceuticals.

### II. Administration

The present disclosure relates to administration of the systems provided herein to subjects. In some embodiments, administration results in the transfection of one or more cells of the subject. In some embodiments, the cells transfected by the systems provided herein are long-lasting cells (e.g., skeletal muscle cells) which result in a steady level of antibody or antigen binding fragment in the subject or antibody or antigen binding fragment production by the cell over time. In some embodiments, this results in maintenance of a therapeutically relevant level of the antibody or antigen binding fragment over time. Such administration resulting in desired or optimal pharmacokinetics of the antibody or antigen binding fragment can be effective for continuous treatment or prevention of the relevant disease. In some embodiments, administration is performed by injection of a lipid vesicle provided herein containing a vector provided herein into a subject.

### Doses

In some embodiments, a prescribed dose of the vector (e.g., a DNA plasmid as provided herein) is administered to a subject. In some embodiments, the prescribed dose is selected in order to elicit a desired level of antibody or antigen binding fragment in the subject, the level of which will depend on the level of antibody or antigen binding fragment which is clinically or therapeutically relevant.

In some embodiments, the dose of vector administered to a subject is 0.1 mg/kg to 20 mg/kg. In some embodiments, the dose of vector administered to a subject is 0.1 mg/kg to 0.5 mg/kg, 0.1 mg/kg to 1 mg/kg, 0.1 mg/kg to 2 mg/kg, 0.1 mg/kg to 3 mg/kg, 0.1 mg/kg to 4 mg/kg, 0.1 mg/kg to 5 mg/kg, 0.1 mg/kg to 7.5 mg/kg, 0.1 mg/kg to 10 mg/kg, 0.1 mg/kg to 20 mg/kg, 0.5 mg/kg to 1 mg/kg, 0.5 mg/kg to 2 mg/kg, 0.5 mg/kg to 3 mg/kg, 0.5 mg/kg to 4 mg/kg, 0.5 mg/kg to 5 mg/kg, 0.5 mg/kg to 7.5 mg/kg, 0.5 mg/kg to 10 mg/kg, 0.5 mg/kg to 20 mg/kg, 1 mg/kg to 2 mg/kg, 1 mg/kg to 3 mg/kg, 1 mg/kg to 4 mg/kg, 1 mg/kg to 5 mg/kg, 1 mg/kg to 7.5 mg/kg, 1 mg/kg to 10 mg/kg, 1 mg/kg to 20 mg/kg, 2 mg/kg to 3 mg/kg, 2 mg/kg to 4 mg/kg, 2 mg/kg to 5 mg/kg, 2 mg/kg to 7.5 mg/kg, 2 mg/kg to 10 mg/kg, 2 mg/kg to 20 mg/kg, 3 mg/kg to 4 mg/kg, 3 mg/kg to 5 mg/kg, 3 mg/kg to 7.5 mg/kg, 3 mg/kg to 10 mg/kg, 3 mg/kg to 20 mg/kg, 4 mg/kg to 5 mg/kg, 4 mg/kg to 7.5 mg/kg, 4 mg/kg to 10 mg/kg, 4 mg/kg to 20 mg/kg, 5 mg/kg to 7.5 mg/kg, 5 mg/kg to 10 mg/kg, 5 mg/kg to 20 mg/kg, 7.5 mg/kg to 10 mg/kg, or 10 mg/kg to 20 mg/kg. In some embodiments, the dose of vector administered to a subject is about 0.1 mg/kg, about 0.5 mg/kg, about 1 mg/kg, about 1.5 mg/kg, 2 mg/kg, about 2.5 mg/kg, about 3 mg/kg, about 3.5 mg/kg, about 4 mg/kg, about 4.5 mg/kg, about 5 mg/kg, about 7.5 mg/kg, about 10 mg/kg or about 20 mg/kg. In some embodiments, the dose of vector administered to a subject is at least 0.1 mg/kg, 0.5 mg/kg, 1 mg/kg, 2 mg/kg, 3 mg/kg, 4 mg/kg, 5 mg/kg, 7.5 mg/kg, or 10 mg/kg. In some embodiments, the dose of vector administered to a subject is at most 0.5 mg/kg, 1 mg/kg, 2 mg/kg, 3 mg/kg, 4 mg/kg, 5 mg/kg, 7.5 mg/kg, 10 mg/kg, or 20 mg/kg. In some embodiments, the subject is administered multiple doses of the same amount of vector. In some embodiments, the subject receives a first dose and a second lower dose (e.g., after a suitable period of time).

In some embodiments, the dose of vector administered to a subject is about 10 micrograms to about 5,000 micrograms. In some embodiments, the dose of vector administered to a subject is about 10 micrograms to about 50 micrograms, about 10 micrograms to about 100 micrograms, about 10 micrograms to about 250 micrograms, about 10 micrograms to about 500 micrograms, about 10 micrograms to about 1,000 micrograms, about 10 micrograms to about 5,000 micrograms, about 50 micrograms to about 100 micrograms, about 50 micrograms to about 250 micrograms, about 50 micrograms to about 500 micrograms, about 50 micrograms to about 1,000 micrograms, about 50 micrograms to about 5,000 micrograms, about 100 micrograms to about 250 micrograms, about 100 micrograms to about 500 micrograms, about 100 micrograms to about 1,000 micrograms, about 100 micrograms to about 5,000 micrograms, about 250 micrograms to about 500 micrograms, about 250 micrograms to about 1,000 micrograms, about 250 micrograms to about 5,000 micrograms, about 500 micrograms to about 1,000 micrograms, about 500 micrograms to about 5,000 micrograms, or about 1,000 micrograms to about 5,000 micrograms. In some embodiments, the dose of vector administered to a subject is about 10 micrograms, about 50 micrograms, about 100 micrograms, about 250 micrograms, about 500 micrograms, about 1,000 micrograms, or about 5,000 micrograms. In some embodiments, the dose of vector administered to a subject is at least about 10 micrograms, about 50 micrograms, about 100 micrograms, about 250 micrograms, about 500 micrograms, or about 1,000 micrograms. In some embodiments, the dose of vector administered to a subject is at most about 50 micrograms, about 100 micrograms, about 250 micrograms, about 500 micrograms, about 1,000 micrograms, or about 5,000 micrograms. In some embodiments, the subject is administered multiple doses of the same amount of vector. In some embodiments, the subject receives a first dose and a second lower dose (e.g., after a suitable period of time).

### Dosing Regimens

In some instances, a dosing regimen is used in order to achieve and/or maintain a desired level of antibody in the subject. In some embodiments, the desired level and duration of antibody level is achieved after a single dose (e.g., for treatment of an acute infection). In some instances, repeat doses (e.g., 2, 3, 4, or more doses) are required in order to achieve an initial therapeutically or clinically relevant level of the antibody or antigen binding fragment (e.g., a higher priming dose or doses followed by a lower maintenance dose).

In some embodiments, the subject is dosed once. In some embodiments, the subject is dosed twice with two weeks between injections. In some embodiments, the subject is dosed twice with three weeks between injections. In some embodiments, the subject is dosed twice with four weeks between injections. In some embodiments, the subject is dosed twice with six weeks between injections. In some embodiments, the subject is dosed twice with eight weeks between injections. In some embodiments, the subject is dosed twice with 12 weeks between injections.

In some instances, the subject is dosed at regularly scheduled intervals (e.g., for continued prophylaxis against an infectious disease, such as a virus). In some embodiments, the subject is dosed approximately once per month, once every two months, once every three months, once every four months, once every six months, or once every year. In some embodiments, the dosing interval is selected such that a minimum level of antibody or antigen binding fragment is consistently achieved (e.g., a blood plasma level in excess of 50 ng/ml, 100 ng/mL, 200 ng/ml, 300 ng/mL, 400 ng/mL, 500 ng/mL, 600 ng/ml, 700 ng/ml, 800 ng/mL, 900 ng/mL, or 1000 ng/mL). In some embodiments, the subject is dosed at regularly scheduled intervals after an initial priming phase (e.g., two or more doses in relatively quick succession, such as about 2-12 week apart).

In instances where multiple doses are administered to a subject, the dose may optionally vary in different doses (e.g., an initial high dose followed by a lower maintenance dose).

In some embodiments, the subject receives multiple doses.

### Routes of Administration

The antibody expression systems provided herein can be administered by a wide variety of routes of administration. In some embodiments, the system is administered by intravenous injection. In some embodiments, the system is administered by subcutaneous injection. In some embodiments, the system is administered by intramuscular injection. In some embodiments, the system is administered by intradermal injection. In some embodiments, the system is administered intranasally. In some embodiments, the system is administered orally. In some embodiments, the system is administered by intrathecal injection. In preferred embodiments, the system is administered by intravenous or intramuscular administration.

In some embodiments, the systems provided herein are capable of being administered and achieving the desired therapeutic effects (e.g., can achieve a required antibody or antigen binding fragment level) without the need of any specialized equipment. In some embodiments, the system is administered without electroporation or hydroporation. In some embodiments, the system is administered without electroporation. In some embodiments, the system is administered without hydroporation. In some embodiments, the system is administered with a standard needle and syringe setup (e.g., for intramuscular administration).

### Activity

In some embodiments, the administered vector is capable of producing plasma antibody or antigen binding fragment concentrations of 10 ng/ml to 20,000 ng/ml. In some embodiments, the administered vector is capable of producing plasma antibody or antigen binding fragment concentrations of 10 ng/ml to 25 ng/ml, 10 ng/ml to 50 ng/ml, 10 ng/ml to 100 ng/ml, 10 ng/ml to 250 ng/ml, 10 ng/ml to 500 ng/ml, 10 ng/ml to 1,000 ng/ml, 10 ng/ml to 2,500 ng/ml, 10 ng/ml to 5,000 ng/ml, 10 ng/ml to 10,000 ng/ml, 10 ng/ml to 15,000 ng/ml, 10 ng/ml to 20,000 ng/ml, 25 ng/ml to 50 ng/ml, 25 ng/ml to 100 ng/ml, 25 ng/ml to 250 ng/ml, 25 ng/ml to 500 ng/ml, 25 ng/ml to 1,000 ng/ml, 25 ng/ml to 2,500 ng/ml, 25 ng/ml to 5,000 ng/ml, 25 ng/ml to 10,000 ng/ml, 25 ng/ml to 15,000 ng/ml, 25 ng/ml to 20,000 ng/ml, 50 ng/ml to 100 ng/ml, 50 ng/ml to 250 ng/ml, 50 ng/ml to 500 ng/ml, 50 ng/ml to 1,000 ng/ml, 50 ng/ml to 2,500 ng/ml, 50 ng/ml to 5,000 ng/ml, 50 ng/ml to 10,000 ng/ml, 50 ng/ml to 15,000 ng/ml, 50 ng/ml to 20,000 ng/ml, 100 ng/ml to 250 ng/ml, 100 ng/ml to 500 ng/ml, 100 ng/ml to 1,000 ng/ml, 100 ng/ml to 2,500 ng/ml, 100 ng/ml to 5,000 ng/ml, 100 ng/ml to 10,000 ng/ml, 100 ng/ml to 15,000 ng/ml, 100 ng/ml to 20,000 ng/ml, 250 ng/ml to 500 ng/ml, 250 ng/ml to 1,000 ng/ml, 250 ng/ml to 2,500 ng/ml, 250 ng/ml to 5,000 ng/ml, 250 ng/ml to 10,000 ng/ml, 250 ng/ml to 15,000 ng/ml, 250 ng/ml to 20,000 ng/ml, 500 ng/ml to 1,000 ng/ml, 500 ng/ml to 2,500 ng/ml, 500 ng/ml to 5,000 ng/ml, 500 ng/ml to 10,000 ng/ml, 500 ng/ml to 15,000 ng/ml, 500 ng/ml to 20,000 ng/ml, 1,000 ng/ml to 2,500 ng/ml, 1,000 ng/ml to 5,000 ng/ml, 1,000 ng/ml to 10,000 ng/ml, 1,000 ng/ml to 15,000 ng/ml, 1,000 ng/ml to 20,000 ng/ml, 2,500 ng/ml to 5,000 ng/ml, 2,500 ng/ml to 10,000 ng/ml, 2,500 ng/ml to 15,000 ng/ml, 2,500 ng/ml to 20,000 ng/ml, 5,000 ng/ml to 10,000 ng/ml, 5,000 ng/ml to 15,000 ng/ml, 5,000 ng/ml to 20,000 ng/ml, 10,000 ng/ml to 15,000 ng/ml, 10,000 ng/ml to 20,000 ng/ml, or 15,000 ng/ml to 20,000 ng/ml. In some embodiments, the administered vector is capable of producing plasma antibody or antigen binding fragment concentrations of 10 ng/ml, 25 ng/ml, 50 ng/ml, 100 ng/ml, 250 ng/ml, 500 ng/ml, 1,000 ng/ml, 2,500 ng/ml, 5,000 ng/ml, 10,000 ng/ml, 15,000 ng/ml, or 20,000 ng/ml. In some embodiments, the administered vector is capable of producing plasma antibody or antigen binding fragment concentrations of at least 10 ng/ml, 25 ng/ml, 50 ng/ml, 100 ng/ml, 250 ng/ml, 500 ng/ml, 1,000 ng/ml, 2,500 ng/ml, 5,000 ng/ml, 10,000 ng/ml, or 15,000 ng/ml.

In some embodiments, the administering produces a peak blood plasma level of the antibody or antigen binding fragment thereof of at least 75 ng/ml, at least 100 ng/ml, at least 150 ng/mL, at least 200 ng/ml, at least 250 ng/mL, at least 300 ng/mL, at least 400 ng/ml, at least 500 ng/mL, at least 600 ng/mL, at least 700 ng/mL, at least 800 ng/ml, at least 900 ng/ml, or at least 1000 ng/mL. In some embodiments, the administering produces a peak blood plasma level of the antibody or antigen binding fragment thereof of at least 1000 ng/ml. In some embodiments, the administering produces a peak blood plasma level of the antibody or antigen binding fragment thereof of at least 1500 ng/mL. In some embodiments, the administering produces a peak blood plasma level of the antibody or antigen binding fragment thereof of at least 2000 ng/mL. In some embodiments, the administering produces a peak blood plasma level of the antibody or antigen binding fragment thereof of at least 2500 ng/ml. In some embodiments, the administering produces a peak blood plasma level of the antibody or antigen binding fragment thereof of at least 3000 ng/mL. In some embodiments, the administering produces a peak blood plasma level of the antibody or antigen binding fragment thereof of at least 4000 ng/mL. In some embodiments, the administering produces a peak blood plasma level of the antibody or antigen binding fragment thereof of at least 5000 ng/ml. In some embodiments, the indicated peak blood plasma level of the antibody or antigen binding fragment is achieved after a single dose of the system provided herein. In some embodiments, the indicated peak blood plasma level of the antibody or antigen binding fragment is achieved after a single intramuscular dose of the system. In some embodiments, the indicated peak blood plasma level of the antibody or antigen binding fragment is achieved after two doses of the system provided herein. In some embodiments, the indicated peak blood plasma level of the antibody or antigen binding fragment is achieved after two intramuscular doses of the system. In some embodiments, the indicated peak blood plasma level of the antibody or antigen binding fragment is achieved after two intravenous doses of the system.

In some embodiments, the antibody or antigen binding fragment blood plasma concentration is maintained at a therapeutically or clinically relevant level (e.g., a level as provided herein, such as a level of at least about 50 ng/mL, 75 ng/mL, 100 ng/mL, 150 ng/ml, 200 ng/ml, 300 ng/mL, 400 ng/mL, 500 ng/mL, 600 ng/ml, 700 ng/ml, 800 ng/ml, 900 ng/mL, 1000 ng/mL, 2000 ng/mL, 3000 ng/mL, 4000 ng/mL, or 5000 ng/ml) for an extended period of time. In some embodiments, the blood plasma level of the antibody or antigen binding fragment is maintained for a period of 1 week to 206 weeks. In some embodiments, the blood plasma antibody or antigen binding fragment concentration is maintained for a period of at least 1 week to 2 weeks, 1 week to 4 weeks, 1 week to 8 weeks, 1 week to 13 weeks, 1 week to 26 weeks, 1 week to 52 weeks, 1 week to 104 weeks, 1 week to 206 weeks, 2 weeks to 4 weeks, 2 weeks to 8 weeks, 2 weeks to 13 weeks, 2 weeks to 26 weeks, 2 weeks to 52 weeks, 2 weeks to 104 weeks, 2 weeks to 206 weeks, 4 weeks to 8 weeks, 4 weeks to 13 weeks, 4 weeks to 26 weeks, 4 weeks to 52 weeks, 4 weeks to 104 weeks, 4 weeks to 206 weeks, 8 weeks to 13 weeks, 8 weeks to 26 weeks, 8 weeks to 52 weeks, 8 weeks to 104 weeks, 8 weeks to 206 weeks, 13 weeks to 26 weeks, 13 weeks to 52 weeks, 13 weeks to 104 weeks, 13 weeks to 206 weeks, 26 weeks to 52 weeks, 26 weeks to 104 weeks, 26 weeks to 206 weeks, 52 weeks to 104 weeks, 52 weeks to 206 weeks, or 104 weeks to 206 weeks. In some embodiments, the blood plasma antibody or antigen binding fragment concentration is maintained for a period of 1 week, 2 weeks, 4 weeks, 8 weeks, 13 weeks, 26 weeks, 52 weeks, 104 weeks, or 206 weeks. In some embodiments, the blood plasma level of the antibody or antigen binding fragment concentration is maintained for a period of at least 1 week, 2 weeks, 4 weeks, 8 weeks, 13 weeks, 26 weeks, 52 weeks, or 104 weeks.

In some embodiments, the antibody or antigen binding fragment blood plasma concentration remains above 50 ng/ml for at least 1 week, 2 weeks, 4 weeks, 8 weeks, 13 weeks, 26 weeks, 52 weeks, or 104 weeks. In some embodiments, the antibody or antigen binding fragment blood plasma concentration remains above 75 ng/ml for at least 1 week, 2 weeks, 4 weeks, 8 weeks, 13 weeks, 26 weeks, 52 weeks, or 104 weeks. In some embodiments, the antibody or antigen binding fragment blood plasma concentration remains above 100 ng/mL for at least 1 week, 2 weeks, 4 weeks, 8 weeks, 13 weeks, 26 weeks, 52 weeks, or 104 weeks. In some embodiments, the antibody or antigen binding fragment blood plasma concentration remains above 250 ng/ml for at least 1 week, 2 weeks, 4 weeks, 8 weeks, 13 weeks, 26 weeks, 52 weeks, or 104 weeks. In some embodiments, the antibody or antigen binding fragment blood plasma concentration remains above 500 ng/ml for at least 1 week, 2 weeks, 4 weeks, 8 weeks, 13 weeks, 26 weeks, 52 weeks, or 104 weeks. In some embodiments, the antibody or antigen binding fragment blood plasma concentration remains above 750 ng/ml for at least 1 week, 2 weeks, 4 weeks, 8 weeks, 13 weeks, 26 weeks, 52 weeks, or 104 weeks. In some embodiments, the antibody or antigen binding fragment blood plasma concentration remains above 1000 ng/mL for at least 1 week, 2 weeks, 4 weeks, 8 weeks, 13 weeks, 26 weeks, 52 weeks, or 104 weeks.

In some embodiments, the blood plasma level of the antibody or antigen binding fragment is sustained at a concentration of at least 50% of the peak blood plasma concentration achieved for a period of at least 4 weeks, at least 6 weeks, at least 8 weeks, at least 10 weeks, at least 20 weeks, at least 30 weeks, or at least 40 weeks after the administration. In some embodiments, the blood plasma level of the antibody or antigen binding fragment is sustained at a concentration of at least 25% of the peak blood plasma concentration achieved for a period of at least 4 weeks, at least 6 weeks, at least 8 weeks, at least 10 weeks, at least 20 weeks, at least 30 weeks, or at least 40 weeks after the administration. In some embodiments, the blood plasma level of the antibody or antigen binding fragment is sustained at a concentration of at least 10% of the peak blood plasma concentration achieved for a period of at least 4 weeks, at least 6 weeks, at least 8 weeks, at least 10 weeks, at least 20 weeks, at least 30 weeks, or at least 40 weeks after the administration.

In some embodiments, the indicated concentrations of antibody or antigen binding fragment is achieved and maintained after a single dose of the vector. In some embodiments, the indicated concentration of antibodies is achieved and maintained after multiple doses of the vector. In some embodiments, the indicated concentration of antibody or antigen binding fragment is achieved and maintained after two doses of the vector. In some embodiments, the antibody or antigen binding fragment concentration is maintained without any additional administration of the vector (e.g., after one or two doses of the vector, depending on the regimen described).

In some embodiments, the subject is administered 2 doses of the vector. In some embodiments, the second dose of the vector is administered from about 2 weeks to about 26 weeks after the first dose. In some embodiments, the 2 doses are administered from about 2 weeks to about 12 weeks apart. In some embodiments, the 2 doses are administered about 2 weeks, about 3 weeks, about 4 weeks, about 5 weeks, about 6 weeks, about 7 weeks, about 8 week, about 9 weeks, about 10 weeks, about 11 weeks, or to about 12 weeks apart. In some embodiments, the 2 doses are administered about 4 weeks to about 12 weeks apart, about 6 weeks to about 12 weeks about, about 8 weeks to about 12 weeks apart, about 4 weeks to about 10 weeks apart, about 6 weeks to about 10 weeks apart, or about 8 weeks to about 10 weeks apart. In some embodiments, the 2 doses are administered at least 2 weeks, at least 4 weeks, or at least 6 weeks apart. In some embodiments, the 2 doses are administered at most 26 weeks apart, at most 20 weeks apart, at most 16 weeks apart, at most 12 weeks apart, or at most 10 weeks apart. In some embodiments, the second dose is administered after a period of plateau of antibody or antigen binding fragment concentration is achieved.

In some embodiments, the 2 doses are the same. In some embodiments, the first dose is higher than the second dose.

In some embodiments, administration of the second dose achieves a peak blood plasma level of the antibody or antigen binding fragment which is higher than a predicted additive effect. In some embodiments, administration of the second dose results in peak blood plasma level of the antibody or antigen binding fragment which is greater than 2-fold higher than the peak blood plasma level achieved after the first dose. In some embodiments, administration of the second dose results in a peak blood plasma level of the antibody or antigen binding fragment which is at least 3-fold, at least 4-fold, or at least 5-fold higher than the peak blood plasma level achieved after the first dose. In some embodiments, administration of the second dose results in a peak blood plasma level of the antibody or antigen binding fragment which is at least 3-fold higher than the peak blood plasma level achieved after the first dose. In some embodiments, administration of the second dose results in a peak blood plasma level of the antibody or antigen binding fragment which is at least 4-fold higher than the peak blood plasma level achieved after the first dose. In some embodiments, administration of the second dose results in a peak blood plasma level of the antibody or antigen binding fragment which is r at least 5-fold higher than the peak blood plasma level achieved after the first dose. In some embodiments, each dose is administered via intravenous administration. In some embodiments, each dose is the same amount, or the second dose is a lower amount than the first dose.

### Subjects

In some embodiments, the subject is an animal. In some embodiments, the subject is a mammal. In some embodiments, the subject is a primate, a feline animal, a canine animal, a bovine animal, a porcine animal, an ovine animal, a caprine animal, or a rodent. In some embodiments, the subject is a human. In some embodiments, the subject is a child or an infant. In some embodiments, the subject is an adult.

### III. Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

Used herein are references to insertions and/or deletions of one or more nucleotides or amino acids from a sequence. As used herein in reference to a sequence, the term “ins” placed before a number followed by a nucleotide or amino acid sequence means that the listed nucleotide or amino acid sequence is inserted into the sequence after the indicated residue. For example, “ins214TDR” indicates that the sequence “TDR” is inserted after residue 214 of the referenced sequence. As used herein, the term “del” following a number or range of numbers indicates that the nucleotide(s) or amino acid(s) at the indicated position numbers of the reference sequence are deleted from the sequence. For example, 137-145del indicates that residues 137, 138, 139, 140, 141, 142, 143, 144, and 145 are deleted from the reference sequence.

The term “V_{H}H” as used herein indicates that the heavy chain variable domain is obtained from or originated or derived from a heavy chain antibody. Heavy chain antibodies are functional antibodies that have two heavy chains and no light chains. Heavy chain antibodies exist in and are obtainable from Camelids (e.g., camels and alpacas), members of the biological family Camelidae. V_{H}H antibodies have originally been described as the antigen binding immunoglobulin (variable) domain of “heavy chain antibodies” (i.e., of “antibodies devoid of light chains”; Hamers-Casterman et al., Nature 363:446-448 (1993). The term “VHH domain” has been chosen in order to distinguish these variable domains from the heavy chain variable domains that are present in conventional four-chain antibodies (which are referred to herein as “VH domains” or “VH”) and from the light chain variable domains that are present in conventional four-chain antibodies (which are referred to herein as “VL domains” or “VL”).

The term “camelized” VH refers to an immunoglobulin single-chain variable domain in which one or more amino acid residues in the amino acid sequence of a naturally occurring VH domain from a conventional four-chain antibody by one or more of the amino acid residues that occur at the corresponding position(s) in a VHH domain of a heavy chain antibody. Such “camelizing” substitutions may be inserted at amino acid positions that form and/or are present at the VH-VL interface, and/or at the so-called Camelidae hallmark residues, as defined herein (see also for example WO9404678 and Davies and Riechmann (1994 and 1996)). Reference is made to Davies and Riechmann (FEBS 339:285-290, 1994; Biotechnol. 13:475-479, 1995; Prot. Eng. 9:531-537, 1996) and Riechmann and Muyldermans (J. Immunol. Methods 231:2538, 1999).

### IV. Sequences

In some embodiments, an antibody or antigen binding fragment in a system provided herein comprises an amino acid sequence having at least 80%, at least 85%, at least 90%, at least 95%, or at least 98% sequence identity to an antibody set forth in the table below.

TABLE 3

| Antibody or | | | |
|---|---|---|---|
| Ag-binding | | | SEQ ID |
| fragment | Region | Sequence | NO |
| Casirivimab | HCVR | QVQLVESGGGLVKPGGSLRLSCAASGFTFSDYY | 1 |
| IMGT/mAb-DB | | MSWIRQAPGKGLEWVSYITYSGSTIYYADSVKG | |
| ID 1105 | | RFTISRDNAKSSLYLQMNSLRAEDTAVYYCARD | |
| Regeneron | | RGTTMVPFDYWGQGTLVTVSS | |
| mAb10933; | HCDR1 | GFTFSDYY | 2 |
| PUB | | | |
| US10787501; | HCDR2 | ITYSGSTI | 3 |
| AP | HCDR3 | ARDRGTTMVPFDY | 4 |
| US20200912678 | LCVR | DIQMTQSPSSLSASVGDRVTITCQASQDITNYLN | 5 |
| [16/912, 678] | | WYQQKPGKAPKLLIYAASNLETGVPSRFSGSGS | |
| (Ab1) | | GTDFTFTISGLQPEDIATYYCQQYDNLPLTFGGG | |
| | | TKVEIK | |
| | LCDR1 | QDITNY | 6 |
| | LCDR2 | AAS | 7 |
| | LCDR3 | QQYDNLPLT | 8 |
| Imdevimab | HCVR | QVQLVESGGGVVQPGRSLRLSCAASGFTFSNYA | 9 |
| IMGT/mAb-DB | | MYWVRQAPGKGLEWVAVISYDGSNKYYADSV | |
| ID 1105 | | KGRFTISRDNSKNTLYLQMNSLRTEDTAVYYCA | |
| Regeneron | | SGSDYGDYLLVYWGQGTLVTVSS | |
| mAb10987; | HCDR1 | GFTFSNYA | 10 |
| PUB | | | |
| US10787501; | HCDR2 | ISYDGSNK | 11 |
| AP | | | |
| US20200912678 | HCDR3 | ASGSDYGDYLLVY | 12 |
| [16/912, 678] | LCVR | QSALTQPASVSGSPGQSITISCTGTSSDVGGYNY | 13 |
| (Ab2) | | VSWYQQHPGKAPKLMIYDVSKRPSGVSNRFSGS | |
| | | KSGNTASLTISGLQSEDEADYYCNSLTSISTWVF | |
| | | GGGTKLTVL | |
| | LCDR1 | SSDVGGYNY | 14 |
| | LCDR2 | DVS | 15 |
| | LCDR3 | NSLTSISTWV | 16 |
| Tixagevimab | HCVR | QMQLVQSGPEVKKPGTSVKVSCKASGFTFM | 17 |
| IMGT/mAb-DB | | SSAVQWVRQARGQRLEWIGWIVIGSGNTNY | |
| ID 1111 | | AQKFQERVTITRDMSTSTAYMELSSLRSED | |
| (Ab3) | | TAVYYCAAPYCSSISCNDGFDIWGQGTMVTVSS | |
| | HCDR1 | GFTFMSSA | 18 |
| | HCDR2 | IVIGSGNT | 19 |
| | HCDR3 | AAPYCSSISCNDGFDI | 20 |
| | LCVR | EIVLTQSPGTLSLSPGERATLSCRASQSVS | 21 |
| | | SSYLAWYQQKPGQAPRLLIYGASSRATGIPDRES | |
| | | GSGSGTDFTLTISRLEPEDFAVYYCQHYGSSRG | |
| | | WT | |
| | LCDR1 | SQSVSSSY | 22 |
| | LCDR2 | GAS | 23 |
| | LCDR3 | QHYGSSRGWT | 24 |
| Cilgavimab | HCVR | EVQLVESGGGLVQPGGSLRLSCAASGFTVSSNY | 25 |
| IMGT/mAb-DB | | MSWVRQAPGKGLEWVSVIYSGGSTFYADSVKG | |
| ID 1106 | | RFTISRDNSMNTLFLQMNSLRAEDTAVYYCARV | |
| (Ab4) | | LPMYGDYLDYWGQGTLVTVSS | |
| | HCDR1 | GFTFRDVW | 26 |
| | HCDR2 | IKSKIDGGTT | 27 |
| | HCDR3 | TTAGSYYYDTVGPGLPEGKFDY | 28 |
| | LCVR | DIVMTQSPDSLAVSLGERAT | 29 |
| | | INCKSSQSVLYSSNNKNYLAWYQQKPGQPPKLL | |
| | | MYWASTRESGVPDRFSGSGSGAEFTLTISSLQAE | |
| | | DVAIYYCQQYYSTLT | |
| | LCDR1 | QSVLYSSNNKNY | 30 |
| | LCDR2 | WAS | 31 |
| | LCDR3 | QQYYSTLT | 32 |
| Ogalvibart | HCVR | QVQLVESGGGVVQPGRSLRLSCAASGFTFSSYA | 33 |
| BMS-986414 | | MHWVRQAPGKGLEWVAVIPFDGRNKYYADSV | |
| (Ab5) | | TGRFTISRDNSKNTLYLQMNSLRAEDTAVYYCA | |
| | | SSSGYLFHSDYWGQGTLVTVSS | |
| | HCDR1 | GFTFSSYA | 34 |
| | HCDR2 | IPFDGRNK | 35 |
| | HCDR3 | ASSSGYLFHSDY | 36 |
| | LCVR | DIQMTQSPSTLSASVGDRVTITCRASQSISNWLA | 37 |
| | | WFQQKPGKAPKLLIYEASSLESGVPSRFSGSGSG | |
| | | TEFTLTISSLQPDDFATYYCQQYNSYPWTFGQG | |
| | | TKVEIK | |
| | LCDR1 | QSISNW | 38 |
| | LCDR2 | EA | 39 |
| | LCDR3 | QQYNSYPWT | 40 |
| Crexavibart | HCVR | EVQLVESGGGLIQPGGSLRLSCAASGFTVSNNY | 41 |
| BMS-986413 | | MSWVRQAPGKGLEWVSVIYSGGSTYYADSVKG | |
| (Ab6) | | RFTISRDKSKNTLYLQMNRLRAEDTAVYYCARE | |
| | | GEVEGYNDFWSGYSRDRYYFDYWGQGTLVTV | |
| | | SS | |
| | HCDR1 | GFTVSNNY | 42 |
| | HCDR2 | IYSGGST | 43 |
| | HCDR3 | AREGEVEGYNDFWSGYSRDRYYFDY | 44 |
| | LCVR | QSALTQPASVSGSPGQSITISCTGTSSDVGGYNY | 45 |
| | | VSWYQQHPGKAPKLMIYDVSNRPSGVSNRFSGS | |
| | | KSGNTASLTISGLQAEDEADYYCSSYTSSSTRVF | |
| | | GTGTKVTVL | |
| | LCDR1 | SSDVGGYNY | 46 |
| | LCDR2 | DV | 47 |
| | LCDR3 | SSYTSSSTRV | 48 |
| Adintrevimab | HCVR | EVQLVESGGGLVKPGGSLRLSCAASGFTFSSYY | 49 |
| ADG20 | | MNWVRQAPGKGLEWVSSISEDGYSTYYPDSLK | |
| IMGT/mAb-DB | | GRFTISRDSAKNSLYLQMNSLRADDTAVYYCAR | |
| ID 1219 | | DFSGHTAWAGTGFEY | |
| (Ab7) | HCDR1 | GFTFSSYY | 50 |
| | HCDR2 | ISEDGYST | 51 |
| | HCDR3 | ARDFSGHTAWAGTGFEY | 52 |
| | LCVR | QSVLTQPPSVSGAPGQRITISCTGSSSNIGAGYDV | 53 |
| | | HWYQQLPGTAPKLLIYGSSSRNSGVPDRFSGSK | |
| | | SGTSASLAITGLQAEDEADYYCQSYDSSLSVLYT | |
| | LCDR1 | SSNIGAGY | 54 |
| | LCDR2 | GSS | 55 |
| | LCDR3 | QSYDSSLSVLYT | 56 |
| MAD0004J08 | HCVR | QVQLVQSGAEVKKPGSSVKVSCKASGGTFSSYT | 57 |
| (Ab8) | | ISWVRQAPGQGLEWMGRIIPILDRVMYAQKFQG | |
| | | RVTITADKSTSTAYMELSSLRSEDTAVYYCARR | |
| | | AIDSDTYVEQSHFDYWGQGTLVTVSS | |
| | HCDR1 | GGTFSSYT | 58 |
| | HCDR2 | IIPILDRV | 59 |
| | HCDR3 | ARRAIDSDTYVEQSHFDY | 60 |
| | LCVR | EIVMTQSPATLSLSPGERATLSCRASQSVSSYLA | 61 |
| | | WYQQKPGQAPSLLIYDASNRATGIPARFSGSGS | |
| | | GTDFTLTISSLEPEDFAVYYCQQPLTFGGGTKVE | |
| | | IK | |
| | LCDR1 | QSVSSY | 62 |
| | LCDR2 | DA | 63 |
| | LCDR3 | QQPLT | 64 |
| Romlusevimab | HCVR | QVQLVQSGSELKKPGASVKVSCKASGYTFTTYV | 65 |
| Brii-198 | | MNWVRQAPGQGLEWMGWINTNTGNPTYAQGF | |
| IMGT/mAb-DB | | TGRFVFSLDTSVSTASLQISSLKAEDTAVYYCSS | |
| ID 1216 | | EITTLGGMDV | |
| (AB9) | HCDR1 | GYTFTTYV | 66 |
| | HCDR2 | INTNTGNP | 67 |
| | HCDR3 | SSEITTLGGMDV | 68 |
| | LCVR | SYVLTQPPSVSVAPGKTARITCGGNNIGSKSVH | 69 |
| | | WYQQKPGQAPVLVIYYDSDRPSGIPERFSGSNS | |
| | | GNTATLTISGVEAGDEADYYCQVWDSISDHRVF | |
| | | GGGTKLTVL | |
| | LCDR1 | NIGSKS | 70 |
| | LCDR2 | YDS | 71 |
| | LCDR3 | QVWDSISDHRV | 72 |
| Amubarvimab | HCVR | EVQLVESGGGLVQPGGSLRLSCAASGITVSSNY | 73 |
| IMGT/mAb-DB | | MNWVRQAPGKGLEWVSLIYSGGSTYYADSVK | |
| ID 1215 | | GRFTISRDNSKNTLYLQMNSLRAEDTAVYHCAR | |
| Brii-196 | | DLVVYGMDVWGQGTTVTVSS | |
| (Ab10) | HCDR1 | GITVSSNY | 74 |
| | HCDR2 | IYSGGST | 75 |
| | HCDR3 | ARDLVVYGMDV | 76 |
| | LCVR | EIVLTQSPGTLSLSPGERATLSCRASQSVSSSYLA | 77 |
| | | WYQQKPGQAPRLLIYGASSRATGIPDRFSGSGS | |
| | | GTDFTLTISRLEPEDFAVYYCQQYGSSPTFGQGT | |
| | | KLEIK | |
| | LCDR1 | SQSVSSSY | 78 |
| | LCDR2 | GAS | 79 |
| | LCDR3 | QQYGSSPT | 80 |
| Enuzovimab | HCVR | EVQLVESGGGLIQPGGSLRLSCAASGFIVSSNYM | 81 |
| IMGT/mAb-DB | | SWVRQAPGKGLEWVSIIYSGGSTFYADSVKGRF | |
| ID 1218 | | TISRDNSKNTLYLQMNSLRVEDTAVYYCARDL | |
| (Ab11) | | QELGSLDYWGQGTLVTVSS | |
| | HCDR1 | GFIVSSNY | 82 |
| | HCDR2 | IYSGGST | 83 |
| | HCDR3 | ARDLQELGSLDY | 84 |
| | LCVR | DIQMTQSPSSVSASVGDRVTITCRASQGISSWLA | 85 |
| | | WYQQKPGKAPKLLIYAASSLQSGVPSRFSGSGS | |
| | | GTDFTLTISSLQPEDFATYYCQEANSFPYTFGQG | |
| | | TKLEIK | |
| | LCDR1 | QGISSWL | 86 |
| | LCDR2 | AAS | 87 |
| | LCDR3 | QEANSFPYT | 88 |
| Lomtegovimab | HCVR | QVQLVESGGGVVQPGRSLRLSCAATGFTFRRYG | 89 |
| DZIF-10c | | MHWVRQAPGKGLEWVAGILFDGSNKYYVDSV | |
| IMGT/mAb-DB | | KGRFTISRDSSRNTLYLQLNSLRREDTAVYYCA | |
| ID 1217 | | KGGDYEWELLESWGQGTLVTVSS | |
| (Ab12) | HCDR1 | GFTFRRYG | 90 |
| | HCDR2 | ILFDGSNK | 91 |
| | HCDR3 | AKGGDYEWELLES | 92 |
| | LCVR | DIQMTQSPSTVSASVGDRVTITCRASQSIDNWLA | 93 |
| | | WYQEKPGKAPKVLIYKASSLESGVPSRFSGRGS | |
| | | GTEFTLTISSLQPGDFATYYCQHYHSFPLTFGGG | |
| | | TKVDIK | |
| | LCDR1 | QSIDNWL | 94 |
| | LCDR2 | KAS | 95 |
| | LCDR3 | QHYHSFPLT | 96 |
| Ty1 (Ab15) | VHH | AQVQLVETGGGLVQPGGSLRLSCAASGFTFSSV | 97 |
| | Variable | YMNWVRQAPGKGPEWVSRISPNSGNIGYTDSV | |
| | | KGRFTISRDNAKNTLYLQMNNLKPEDTALYYC | |
| | | AIGLNLSSSSVRGQGTQVTVSS | |
| | CDR1 | GFTFSSVYM | 98 |
| | CDR2 | RISPNSGNIG | 99 |
| | CDR3 | AIGLNLSSSSV | 100 |

Although the present disclosure and its advantages have been described in detail, it should be understood that various changes, substitutions and alterations can be made herein without departing from the spirit and scope of the disclosure as defined in the appended claims. The present disclosure is further illustrated in the following Examples which are given for illustration purposes only and are not intended to limit the disclosure in any way.

### EXAMPLES

### Example 1—Antibody or Antigen Binding Fragment Expression System Design

Monoclonal antibody (mAb) sequences were constructed as either single-transcript (ST) or heavy chain/light chain (HC+LC) formats. Antibodies in the ST format were of one of two types: Furin T2A (T2A) linked heavy chain (HC) and light chain (LC) or V_{H}H format. Antibody encoding DNA sequences were codon optimized using the Integrated DNA Technologies (IDT) codon optimization web tool (strategy 1) or the ThermoFisher GeneOptimizer™ web tool (strategy 2).

The T2A format was designed by fusing nucleotide sequences encoding the following elements in order: Kozak sequence; HC signal peptide; immunoglobulin HC; furin cleavage site; T2A peptide derived from Thosea asigna virus; LC signal peptide; immunoglobulin LC; and stop codon. Antibody encoding DNA sequences were codon optimized using the Integrated DNA Technologies (IDT) codon optimization web tool (strategy 1) or the ThermoFisher GeneOptimizer™ web tool (strategy 2) to reduce rare codon usage, balance GC content, and minimize RNA secondary structures. All immunoglobulin HCs used the IGHG1*01 gene sequence, and LC used either the IGKC*01 (for mAb1) or IGLC2*01 (for mAb2) genes. The full open reading frame was preceded by a CAG promoter. FIG. 5A shows an exemplary vector map of such a sequence.

The HC+LC formats were constructed with two plasmids (one encoding the HC and one encoding the LC) using the following elements in order: Kozak sequence; signal peptide (for either HC or LC); immunoglobulin HC or LC; and stop codon. As in the ST format the open reading frame was preceded by a CAG promoter and followed by a BGH polyadenylation signal. In some heavy chain sequences the YTE mutation [M252Y, S254T, T256E (EU numbering)] was introduced to increase serum/plasma half-life. FIGS. 5B and 5C show exemplary vector maps of such sequences.

V_{H}H antibody constructs were designed using the following sequences in order: Kozak sequence; HC signal peptide; V_{H}H variable domain sequence; modified human hinge region; human CH2 and CH3 domains from IGHG1*01; and stop codon. The open reading frame was preceded by a CAG promoter and followed by a BGH poly-adenylation signal. The V_{H}H variable domain sequence used in this study is Ty1, an anti-SARS-COV-2 V_{H}H isolated and published in Hanke, et al., Nat. Comms. 2020 (doi: 10.1038/s41467-020-18174-5)

The V_{H}H, T2A and HC+LC formats were constructed as circular nanoplasmids (Nature Technology Corporation). The nanoplasmids include, in addition to the elements mentioned above, an RNA-OUT selectable marker plus R6K origin to allow propagation in bacterial hosts. The nanoplasmid is sold commercially by Nature Technology Corporation under the trade name Nanoplasmid™.

TABLE 4

| Ab | mAb | Fc | LC |
|---|---|---|---|
| Number | Name | Modification | type |
| Ab1 | Casirivimab | None | Kappa |
| Ab2 | Imdevimab | None | Lambda |
| Ab3 | Tixagevimab | YTE | Kappa |
| Ab4 | Cilgavimab | YTE | Kappa |
| Ab5 | BMS-986414 | YTE | Kappa |
| Ab6 | BMS-986413 | YTE | Lambda |
| Ab7 | ADG-20 | YTE | Lambda |
| Ab8 | MAD-0004J08 | YTE | Kappa |
| Ab9 | Brii-198 | YTE | Kappa |
| Ab10 | Brii-196 | YTE | Lambda |
| Ab11 | Enuzovimab | YTE | Kappa |
| Ab12 | DZIF-10c | YTE | Kappa |
| Ab15 | Ty 1 | YTE | N/A |

### Example 2—In Vitro Testing of DNA Encoded Antibodies

DNA encoded antibody candidates described in Example 2 were tested for expression in vitro to verify protein production. HEK293T cells were seeded at a density of 2×10⁵ cells per well in a 12 well plate in Dulbecco's Modified Eagle Media, supplemented with 10% fetal bovine serum and penicillin-streptomycin. One day after seeding, cells were transfected using 3.75 μl Lipofectamine 3000, 2 μl P3000, and 1 μg DNA per well. Plasmid DNA encoding GFP was transfected in parallel with each batch of mAb candidates, and GFP fluorescence was measured 24 hours post-transfection as a control. Supernatant for mAb transfections was collected after 48 or 72 hours to measure IgG titers, which were quantified using sandwich ELISA. Plates were coated overnight with goat anti-human Fc polyclonal antibody and human IgG in supernatant was detected using goat anti-human H+L polyclonal antibody coupled to horseradish peroxidase. Candidates were tested in the following configurations: 1) T2A nanoplasmids, 2) HC+LC nanoplasmids, and 3) T2A plasmids. IgG expression values are reported as the mean of two duplicates. Samples were diluted 1:10, 1:50, 1:250, and 1:1250 to accurately quantify titers compared to a standard curve of purified human IgG1, at concentrations ranging from 3 μg/ml-1.3 ng/ml. The data provided below was generated using a commercially available human IgG1 standard (IgG1 Human ELISA Standard (for Uncoated ELISA Kit) from ThermoFisher Scientific, Cat. No. 39-50560-65). All samples described herein as measured using a commercial standard refer to this same standard. Standard curves were fit using nonlinear regression to a four-parameter sigmoidal dose-response curve, and the dilution of cell supernatant which was in the linear dynamic range of the ELISA was used to interpolate the concentration. Reported IgG expression values are the mean of two biological replicates.

TABLE 5
In Vitro Expression Dosage, Codon Optimization, Antibody Format, and Results

| | | | | Heavy chain/ | | Codon | | IgG |
|---|---|---|---|---|---|---|---|---|
| Iteration | | mAb | Antibody | light chain | | Optimization | Dose | expression |
| # | Vector | candidate | format | format | Promoter | Strategy | (μg) | (μg/ml) |
| 1 | Nanoplasmid | Ab1 | Human | T2A | CAG | 1 | 4 | 8.9 |
| | | | IgG1 | | | | | |
| 2 | Nanoplasmid | Ab1 | Human | T2A | CAG | 1 | 10 | 6.5 |
| | | | IgG1 | | | | | |
| 3 | Nanoplasmid | Ab1 | Human | HC + LC | CAG | 1 | 4 | 143.5 |
| | | | IgG1 | | | | | |
| 4 | Nanoplasmid | Ab1 | Human | HC + LC | CAG | 1 | 10 | 183.8 |
| | | | IgG1 | | | | | |
| 5 | Nanoplasmid | Ab2 | Human | T2A | CAG | 1 | 4 | 8.9 |
| | | | IgG1 | | | | | |
| 6 | Nanoplasmid | Ab2 | Human | T2A | CAG | 1 | 10 | 11.1 |
| | | | IgG1 | | | | | |
| 7 | Nanoplasmid | Ab2 | Human | HC + LC | CAG | 1 | 4 | 226.9 |
| | | | IgG1 | | | | | |
| 8 | Nanoplasmid | Ab2 | Human | HC + LC | CAG | 1 | 10 | 34.6 |
| | | | IgG1 | | | | | |
| 9 | Plasmid | Ab1 | Human | T2A | CAG | 2 | 1 | 116.1 |
| | | | IgG1 | | | | | |
| 10 | Plasmid | Ab2 | Human | T2A | CAG | 2 | 1 | 127.4 |
| | | | IgG1 | | | | | |
| 11 | Plasmid | Ab1 | Human | T2A | CAG | 2 | 1 | 40.3 |
| | | | IgG1 | | | | | |
| 12 | Plasmid | Ab2 | Human | T2A | CAG | 2 | 1 | 49.9 |
| | | | IgG1 | | | | | |
| 13 | Plasmid | Ab3 | Human | T2A | CAG | 2 | 1 | 198.1 |
| | | | IgG1 | | | | | |
| 14 | Plasmid | Ab4 | Human | T2A | CAG | 2 | 1 | 127.2 |
| | | | IgG1 | | | | | |
| 15 | Plasmid | Ab5 | Human | T2A | CAG | 2 | 1 | 89.1 |
| | | | IgG1 | | | | | |
| 16 | Plasmid | Ab6 | Human | T2A | CAG | 2 | 1 | 173.8 |
| | | | IgG1 | | | | | |
| 17 | Plasmid | Ab7 | Human | T2A | CAG | 2 | 1 | 95.5 |
| | | | IgG1 | | | | | |
| 18 | Plasmid | Ab8 | Human | T2A | CAG | 2 | 1 | 36.0 |
| | | | IgG1 | | | | | |
| 19 | Plasmid | Ab9 | Human | T2A | CAG | 2 | 1 | 6.9 |
| | | | IgG1 | | | | | |
| 20 | Plasmid | Ab10 | Human | T2A | CAG | 2 | 1 | 59.3 |
| | | | IgG1 | | | | | |
| 21 | Plasmid | Ab11 | Human | T2A | CAG | 2 | 1 | 45.0 |
| | | | IgG1 | | | | | |
| 22 | Plasmid | Ab12 | Human | T2A | CAG | 2 | 1 | 69.8 |
| | | | IgG1 | | | | | |
| 23 | Plasmid | Ab15 | V_{H}H - Fc | ST | CAG | 2 | 1 | 687.9 |
| | | | fusion | | | | | |
| 24 | Plasmid | Ab2 | Human | T2A | CAG | 2 | 1 | 33.7 |
| | | | IgG1 | | | | | |
| 25 | Plasmid | Ab15 | V_{H}H - Fc | ST | CAG | 2 | 1 | 41.3 |
| | | | fusion | | | | | |
| 26 | mRNA | Ab2 | Human | T2A | β-globin | 2 | 1 | 0.3 |
| | | | IgG1 | | UTR | | | |
| 27 | mRNA | Ab15 | V_{H}H - Fc | ST | β-globin | 2 | 1 | 4.9 |
| | | | fusion | | UTR | | | |

### Example 3—In Vivo Expression Testing Study 1

Proteo-lipid vesicles (PLVs) containing the T2A nanoplasmids were formulated to concentrations of 2.5, 2, 1, 0.6, and 0.33 mg/ml. PLVs containing the co-formulated HC+LC nanoplasmids were generated at a total concentration of 1 mg/ml (0.5 mg/ml HC nanoplasmid and 0.5 mg/ml LC nanoplasmid).

An exemplary process to manufacture the PLVs is as follows: The plasmid DNA species is encapsulated within fusion-associated small transmembrane protein (FAST)-PLVs as payload. Plasmid DNA is diluted in 10 mM sodium acetate buffer (pH 4.0) containing 5 nM FAST protein (Fusogenix from Entos Pharmaceuticals, San Diego, CA). Separately, the PLV lipid components are dissolved in ethanol. Mixing the DNA-protein fraction with the lipid fraction is performed in the NanoAssemblr Benchtop microfluidics instrument (Precision Nanosystems Inc, Vancouver, BC) at a 3:1 ratio and a flow rate of 12 mL/min. Formulations are dialyzed in 8000 MWCO dialysis membranes (product code 12757486, BioDesign, Carmel, New York) against phosphate buffered saline (pH 7.4) for 3 hours with three buffer changes, then concentrated using Amicon ultracentrifuge filters (EMD Millipore, Burlington, Massachusetts) before passage through a 0.22 μm filter (GSWP04700, EMD Millipore). The resulting FAST-PLV DNA species are stored at 4° C. until used.

Rag2 knockout mice were used to study antibody expression and titers due to their inability to mount an immune response against human antibodies. To optimize the in vivo antibody expression, a comparison was made between different vector strategies (T2A vs. HC+LC), doses and routes of administration, Intravenous (IV) vs. Intramuscular (IM). and followed by a BGH poly-adenylation signal.

TABLE 6
Mouse Injections with PLVs including plasmid DNA Dosages, Vector
Format, Administration Routes, and Injection Volumes.

| No. | Cargo | Vector | Dose | Volume | Route |
|---|---|---|---|---|---|
| 1 | Vehicle only | NA | NA | 100 μl | IV |
| 2 | Ab1 | T2A | 250 μg | 100 μl | IV |
| 3 | Ab1 | T2A | 100 μg | 100 μl | IV |
| 4 | Ab1 | HC + LC | 100 μg total (50 μg | 100 μl | IV |
| | | | each nanoplasmid) | | |
| 5 | Ab2 | T2A | 250 μg | 100 μl | IV |
| 6 | Ab2 | T2A | 100 μg | 100 μl | IV |
| 7 | Ab2 | HC + LC | 100 μg total (50 μg | 100 μl | IV |
| | | | each nanoplasmid) | | |
| 8 | Vehicle only | NA | NA | 50 μl | IM |
| 9 | Ab1 | T2A | 100 μg | 50 μl | IM |
| 10 | Ab1 | T2A | 30 μg | 50 μl | IM |
| 11 | Ab2 | T2A | 100 μg | 50 μl | IM |
| 12 | Ab2 | T2A | 30 μg | 50 μl | IM |
| 13 | Ab1 + Ab2 | T2A | 100 μg total (50 μg | 50 μl Ab1 T2A in left | IM |
| | | | of each Ab in | flank, 50 μl Ab2 T2A | |
| | | | opposing flanks) | in right flank | |
| 14 | Ab1 + Ab2 | T2A | 30 μg total (15 μg | 50 μl Ab1 T2A in left | IM |
| | | | of each Ab in | flank, 50 μl Ab2 T2A | |
| | | | opposing flanks) | in right flank | |
| 15 | Ab1 | HC + LC | 500 μg total (250 μg | 100 μl | IV |
| | | | each nanoplasmid) | | |
| 16 | Ab1 | HC + LC | 500 μg total (250 μg | 100 μl (50 1 each | IM |
| | | | each nanoplasmid) | plasmid) | |
| 17 | Ab1 | HC + LC | 250 μg total (125 μg | 100 μl | IV |
| | | | each nanoplasmid) | | |
| 18 | Ab1 | HC + LC | 250 μg total (125 μg | 100 μl (50 1 each | IM |
| | | | each nanoplasmid) | plasmid) | |

Blood samples were collected and processed to plasma at: various timepoints as indicated herein. Human IgG titers are measured in mouse plasma by electro-chemiluminescence assay (ECLIA) using a Meso Scale Discovery instrument. Human IgG titers in mice are quantified by measuring ECLIA signal of plasma samples diluted 1:100 and interpolated based on a standard curve of purified human IgG1, at concentrations ranging from 3.2 μg/ml-0.78 ng/ml. The data provided in Table 7 below was generated using a commercially available human IgG1 standard. Standard curves are fit using nonlinear regression to a four-parameter sigmoidal dose-response curve. Human IgG expression values reported are the mean of each group at day 23 post-injection.

TABLE 7

| | | | Heavy chain/ | | Codon | | | Mean human |
|---|---|---|---|---|---|---|---|---|
| | | mAb | light chain | | Optimization | Dose | Administration | IgG expression |
| Iteration# | Vector | candidate | format | Promoter | Strategy | (μg) | route | (ng/ml) |
| 2 | Nanoplasmid | Ab1 | T2A | CAG | 1 | 250 | IV | 400.1 |
| | | | | | | | (Intravenous) | |
| 3 | Nanoplasmid | Ab1 | T2A | CAG | 1 | 100 | IV | 414.8 |
| 4 | Nanoplasmid | Ab1 | HC + LC | CAG | 1 | 100 | IV | 1023.9 |
| 5 | Nanoplasmid | Ab2 | T2A | CAG | 1 | 250 | IV | 63.6 |
| 6 | Nanoplasmid | Ab2 | T2A | CAG | 1 | 100 | IV | 0.0 |
| 7 | Nanoplasmid | Ab2 | HC + LC | CAG | 1 | 100 | IV | 1093.9 |
| 9 | Nanoplasmid | Ab1 | T2A | CAG | 1 | 100 | IM | 529.1 |
| | | | | | | | (Intramuscular) | |
| 10 | Nanoplasmid | Ab1 | T2A | CAG | 1 | 30 | IM | 226.3 |
| 11 | Nanoplasmid | Ab2 | T2A | CAG | 1 | 100 | IM | 46.6 |
| 12 | Nanoplasmid | Ab2 | T2A | CAG | 1 | 30 | IM | 0.0 |
| 13 | Nanoplasmid | Ab1 + Ab2 | T2A | CAG | 1 | 100 | IM | 376.6 |
| 14 | Nanoplasmid | Ab1 + Ab2 | T2A | CAG | 1 | 30 | IM | 0.0 |
| 15 | Nanoplasmid | Ab1 | HC + LC | CAG | 1 | 500 | IV | 4526.4 |
| 16 | Nanoplasmid | Ab1 | HC + LC | CAG | 1 | 500 | IM | |
| 17 | Nanoplasmid | Ab1 | HC + LC | CAG | 1 | 250 | IM | 2232.2 |
| 18 | Nanoplasmid | Ab1 | HC + LC | CAG | 1 | 250 | IM | 1551.2 |

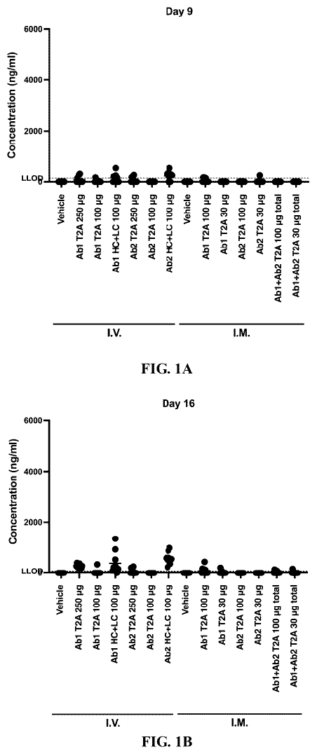

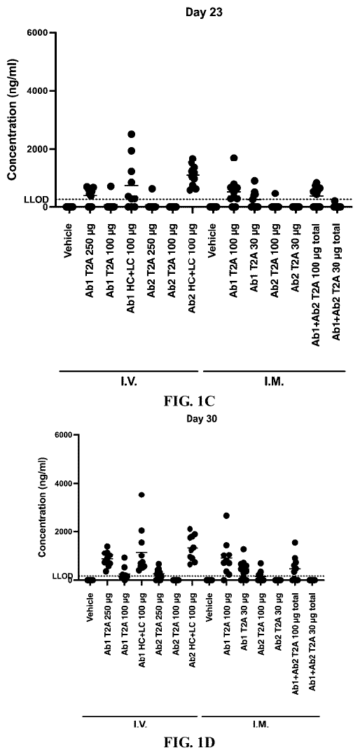

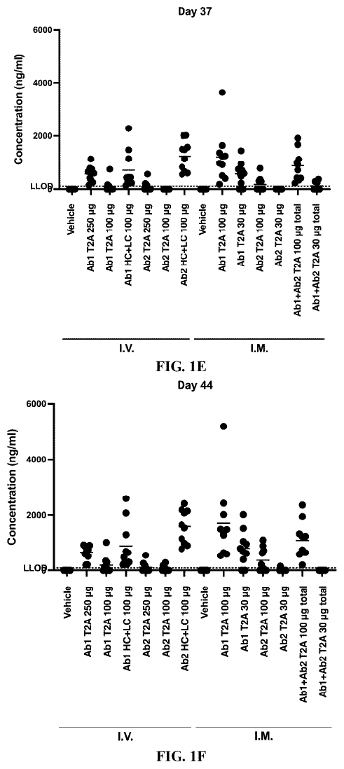

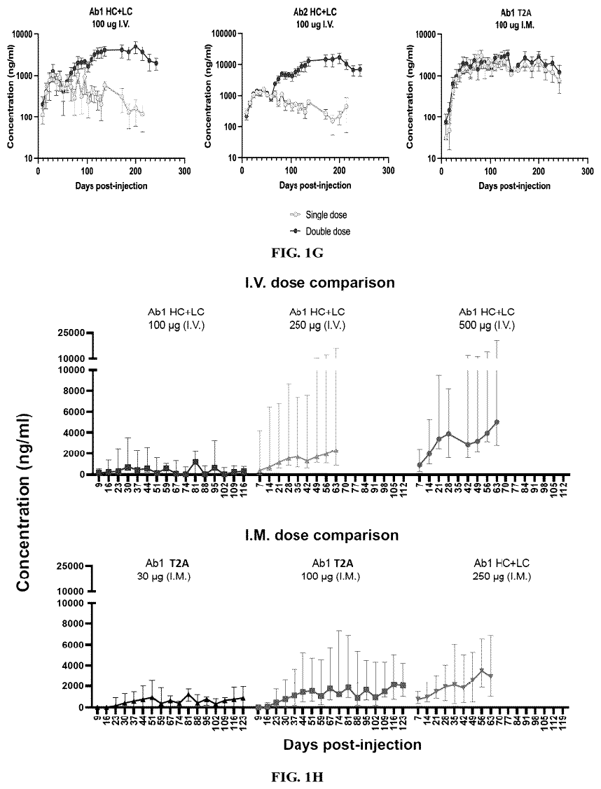

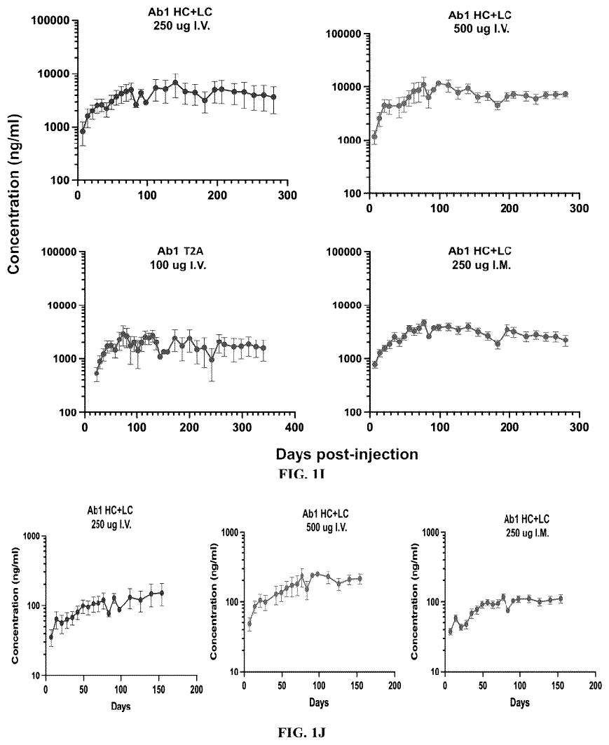

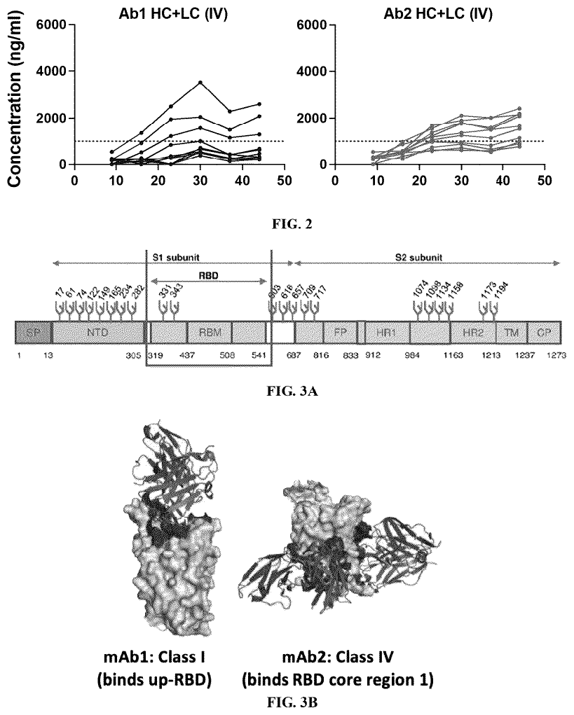

FIG. 1A shows IgG blood plasma levels in mice at day 9 after administration of the indicated construct. FIG. 1B shows IgG blood plasma levels in mice at day 16 after administration of the indicated construct. FIG. 1C shows IgG blood plasma levels in mice at day 23 after administration of the indicated construct. FIG. 1D shows IgG blood plasma levels in mice at day 30 after administration of the indicated construct. FIG. 1E shows IgG blood plasma levels in mice at day 37 after administration of the indicated construct. FIG. 1F shows IgG blood plasma levels in mice at day 44 after administration of the indicated construct. FIG. 2 shows IgG concentrations in blood plasma from mice for the Ab1 HC+LC and Ab2 HC+LC format administered via intravenous administration (entries 4 and 7 of Table 6) at various time points for individual animals. The data provided in each of FIGS. 1A-IF and FIG. 2 was generated using a commercially available human IgG1 standard.

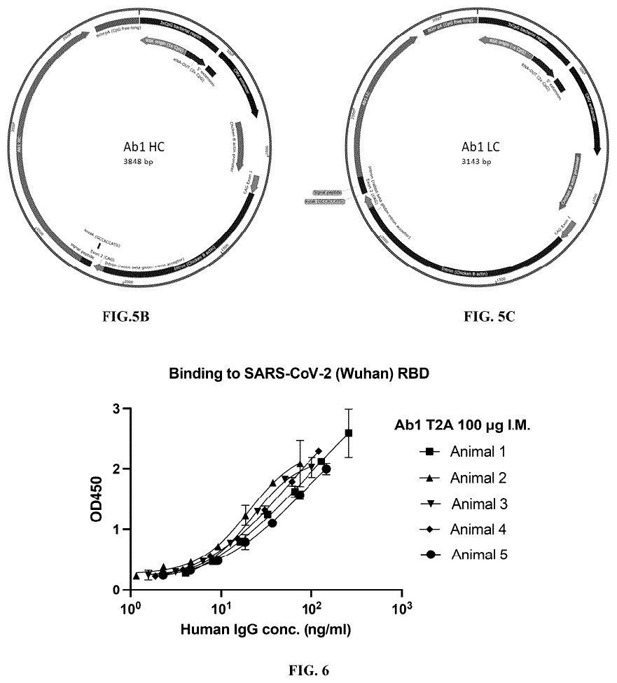

At the day 44 post-injection time point, mouse plasma as assessed for binding to SARS-CoV-2 (Wuhan) RBD protein. Binding was measured by sandwich ELISA, in which ELISA plates were coated overnight with commercially available SARS-COV-2 RBD protein (SinoBiological) at a concentration of 1 μg/ml. Plasma samples from five mice given 100 μg of Ab1 in T2A format administered via intramuscular route (No. 11 from tables 6 and 7) were assessed. Plasma samples were incubated with RBD-coated plates at dilution factors of 1:10, 1:20, 1:40, 1:80, 1:160, 1:320, and 1:640. Binding was detected using goat anti-human Fc polyclonal antibody coupled to horseradish peroxidase. Results of this experiment are shown in FIG. 6, with the concentrations of antibody on the x-axis determined as calculated from the dilution ratio based on initial IgG concentration using a commercially available human IgG1 standard.

Table 8 below shows human IgG concentrations in units of ng/ml in mice administered the system for antibody or antigen binding fragment described in experiment No. 15 in Table 6 above at various time points. The data provided below was generated using a commercially available human IgG1 standard.

TABLE 8

| Days post- | | | | | |
|---|---|---|---|---|---|
| injection | Ab1 HC + LC 500 ug IV (15) | | | | |
| 9 | 888.1371 | 1373.909 | 298.0508 | 2417.572 | 879.0049 |
| 16 | 1947.492 | 2512.397 | 1004.949 | 5211.666 | 1943.725 |
| 23 | 3179.766 | 4094.447 | 2446.367 | 9526.474 | 3385.108 |
| 30 | | 3730.58 | 1566.184 | 4012.906 | 8173.375 |

Table 9 below shows human IgG concentrations in units of ng/ml in mice administered the system for antibody or antigen binding fragment described in experiment No. 4 in Table 6 above at various time points. The data provided below was generated using a commercially available human IgG1 standard.

TABLE 9

| Days post- | | | | | | | | | | |
|---|---|---|---|---|---|---|---|---|---|---|
| injection | Ab1 HC + LC 100 ug IV (4) | | | | | | | | | |
| 9 | 163.614 | 0 | 0 | 0 | 0 | 224.2553 | 553.9434 | 212.2685 | 259.8615 | 188.0933 |
| 16 | 87.66045 | 163.614 | 283.3358 | 138.7752 | 531.9848 | 927.2451 | 1346.667 | 212.2685 | 0 | 224.2553 |
| 23 | 370.4406 | 849.967 | 0 | 0 | 1218.863 | 1942.315 | 2493.977 | 0 | 277.404 | 285.1099 |
| 30 | 553.3126 | 1000.199 | 553.3126 | 396.838 | 1556.184 | 2040.679 | 3522.322 | 724.1274 | 502.9296 | 668.7701 |
| 37 | 282.3219 | 428.4936 | 221.6768 | 131.6658 | 1147.02 | 1481.329 | 2283.451 | 445.6516 | 232.0992 | 437.0976 |
| 44 | 292.0497 | 682.9683 | 205.2003 | 215.2681 | 1286.019 | 2075.729 | 2589.418 | 328.4704 | 473.4672 | 638.032 |
| 51 | 9.06336 | 377.2366 | 34.41576 | 26.53277 | 950.6376 | 1146.747 | 1596.244 | 63.4282 | 0 | 263.5723 |
| 59 | 566.8306 | 796.7689 | 149.4784 | 129.4041 | 1029.339 | 986.4305 | | | | 342.1275 |
| 67 | 97.04587 | | | 89.02635 | 1332.867 | 1061.924 | | | | 45.38453 |
| 74 | 0 | | | 0 | 738.4998 | 704.8348 | | | | 0 |
| 81 | 0 | | | 1249.782 | 1202.234 | 457.9019 | | | | 2194.539 |
| 88 | 0 | | | 773.0208 | 0 | 0 | | | | 802.5818 |

Table 10 below shows human IgG concentrations in units of ng/ml in mice administered the system for antibody or antigen binding fragment described in experiment No. 7 in Table 6 above at various time points. The data provided below was generated using a commercially available human IgG1 standard.

TABLE 10

| Days post- | | | | | | | | | | |
|---|---|---|---|---|---|---|---|---|---|---|
| injection | Ab2 HC + LC 100 ug IV (7) | | | | | | | | | |
| 9 | 306.6241 | 224.2553 | 0 | 553.9434 | 318.2038 | 259.8615 | 271.6231 | 0 | 283.3358 | 329.743 |
| 16 | 520.9729 | 465.5644 | 979.2446 | 586.7257 | 874.9631 | 236.1807 | 542.9748 | 364.1341 | 586.7257 | 553.9434 |
| 23 | 590.0371 | 1054.628 | 1530.433 | 1219.053 | 1170.436 | 743.5217 | 1356.506 | 629.3956 | 1669.465 | 975.3535 |
| 30 | 720.2615 | 1247.745 | 2106.502 | 1752.119 | 1351.645 | 884.2224 | 1799.248 | 629.1979 | 1892.376 | 942.2168 |
| 37 | 549.9825 | 1139.967 | 2019.102 | 1575.436 | 1488.098 | 658.5494 | 1505.689 | 597.252 | 2008.398 | 825.9939 |
| 44 | 983.4027 | 1528.613 | 2144.248 | 2186.587 | 1641.054 | 898.9387 | 2077.226 | 779.913 | 2406.659 | 1139.218 |
| 51 | 825.9476 | 1091.618 | 1750.965 | 1176.453 | 1182.452 | 792.9587 | 1295.048 | 766.3164 | 1433.849 | 998.8316 |
| 59 | 630.4914 | 873.8886 | 1331.322 | 1084.534 | 941.6988 | 428.9667 | 947.2919 | 476.306 | 1492.678 | 580.3021 |
| 67 | 901.0645 | | | 1289.178 | 1459.559 | | | 317.7143 | 1827.528 | |
| 74 | 448.0485 | | | 804.7319 | 672.0679 | | | 179.6538 | 776.7798 | |
| 81 | 976.215 | | | 1319.345 | 1351.658 | | | 725.2426 | 1154.214 | |
| 88 | 395.4981 | | | 0 | 450.8104 | | | 674.3348 | 1087.032 | |

Table 11 below shows human IgG concentrations in units of ng/ml in mice administered the system for antibody or antigen binding fragment described in experiment No. 9 in Table 6 above at various time points. The data provided below was generated using a commercially available human IgG1 standard.

TABLE 11

| Days post- | | | | | | | | | | |
|---|---|---|---|---|---|---|---|---|---|---|
| injection | Ab1 SC 100 ug IM (9) | | | | | | | | | |
| 9 | 188.0933 | 0 | 188.0933 | 0 | 0 | 0 | 0 | 163.614 | 0 | 0 |
| 16 | 443.2258 | 175.8947 | 0 | 151.2438 | 0 | 0 | 0 | 0 | 87.66045 | 126.1976 |
| 23 | 1698.818 | 529.5211 | 668.0556 | 792.6162 | 0 | 281.0669 | 0 | 361.389 | 678.0895 | 281.0669 |
| 30 | 2665.516 | 1023.194 | 790.5839 | 1445.936 | 227.1308 | 693.3393 | 0 | 684.2929 | 1039.147 | 346.2292 |
| 37 | 3638.764 | 970.1374 | 1253.058 | 1638.544 | 399.5472 | 916.0572 | 185.2317 | 1369.117 | 1234.434 | 501.4261 |
| 44 | 5190.242 | 1481.627 | 2015.755 | 2418.387 | 638.318 | 1468.124 | 532.7421 | 1344.928 | 1268.037 | 586.2118 |
| 51 | 4690.396 | 1602.736 | 1970.232 | 2265.278 | 845.5816 | 1347.519 | 586.729 | 1518.886 | 1723.761 | 786.3196 |
| 59 | 4551.987 | 986.2095 | 1906.226 | 2359.663 | 489.6061 | 1068.311 | 238.2392 | 1019.254 | 1248.748 | 456.1736 |
| 67 | 5692.375 | | | | 702.773 | 1486.652 | | 1806.242 | 1763.457 | |
| 74 | 3899.179 | | | | 7334.743 | 1112.575 | | 1191.957 | 1061.692 | |
| 81 | 2027.857 | | | | 6886.101 | 1664.016 | | 874.6619 | 1858.288 | |
| 88 | 1576.567 | | | | 882.3472 | 197.8782 | | 5344.136 | 613.6832 | |

Table 12 below shows human IgG concentrations in units of ng/mL in mice administered the system for antibody or antigen binding fragment described in experiment No. 10 in Table 6 above at various time points. The data provided below was generated using a commercially available human IgG1 standard.

TABLE 12

| Days post- | | | | | | | | | | |
|---|---|---|---|---|---|---|---|---|---|---|
| injection | Ab1 SC 30 ug IM (10) | | | | | | | | | |
| 9 | 0 | 0 | 0 | 0 | 0 | 0 | 0 | 0 | 0 | 0 |
| 16 | 87.66045 | 0 | 0 | 0 | 0 | 0 | 0 | 212.2685 | 0 | 0 |
| 23 | 399.6711 | 523.055 | 0 | 0 | 0 | 0 | 255.7375 | 902.8 | 0 | 436.9504 |
| 30 | 693.3393 | 357.385 | 0 | 300.4073 | 130.4243 | 0 | 647.7244 | 1285.151 | 443.1446 | 572.5526 |
| 37 | 745.6519 | 557.3186 | 0 | 230.832 | 480.396 | 0 | 702.2773 | 1443.146 | 585.3153 | 944.2487 |
| 44 | 1511.929 | 669.4991 | 0 | 389.4866 | 608.7636 | 0 | 951.4967 | 2018.862 | 795.5632 | 1048.159 |
| 51 | 1085.496 | 759.6195 | 114.5755 | 1017.558 | 691.7934 | 0 | 822.3573 | 2580.48 | 1073.225 | 1224.237 |
| 59 | 730.5363 | 354.3574 | 0 | 345.0006 | 87.49763 | 0 | 522.459 | 1812.625 | 226.3515 | 644.5341 |
| 67 | 1084.965 | 612.3065 | | 630.0367 | | 0 | 800.5912 | | | |
| 74 | 570.4287 | 375.7465 | | 294.8209 | | 97.19909 | 381.8508 | | | |
| 81 | 1209.957 | 1340.911 | | 813.3544 | | 566.4843 | 1749.445 | | | |
| 88 | 294.914 | 1199.9 | | 0 | | 395.4981 | 738.0504 | | | |

Table 13 below shows human IgG concentrations in units of ng/ml in mice administered the system for antibody or antigen binding fragment described in experiment No. 17 in Table 6 above at various time points. The data provided below was generated using a commercially available human IgG1 standard.

TABLE 13

| Days post- | | | | | | | | | | |
|---|---|---|---|---|---|---|---|---|---|---|
| injection | Ab1 HC + LC 250 ug IV (17) | | | | | | | | | |
| 9 | 344.6724 | 170.3028 | 849.7412 | 0 | 0 | 227.1528 | 682.4233 | 4194.57 | 432.2618 | 1485.948 |
| 16 | 438.7013 | 596.343 | 808.6671 | 512.7186 | 387.8848 | 576.6677 | 1256.672 | 6496.643 | 1441.783 | 3417.753 |
| 23 | 995.4223 | 2080.348 | 1167.721 | 945.7953 | 614.9658 | 1023.489 | 1133.825 | 9026.614 | 1228.094 | 4105.403 |
| 30 | 1940.701 | 1096.967 | 1244.56 | 1393.248 | 855.5201 | 2448.58 | 1055.654 | 1700.916 | 5576.625 | 8639.646 |

Table 14 below shows human IgG concentrations in units of ng/ml in mice administered the system for antibody or antigen binding fragment described in experiment No. 18 in Table 6 above at various time points. The data provided below was generated using a commercially available human IgG1 standard.

TABLE 14

| Days post- | | | | | | | | | | |
|---|---|---|---|---|---|---|---|---|---|---|
| injection | Ab1 HC + LC 250 ug IM (18) | | | | | | | | | |
| 9 | 520.1523 | 756.2823 | 382.1388 | 710.3919 | 858.5976 | 1095.042 | 1087.336 | 577.6254 | 1504.558 | 329.092 |
| 16 | 910.3836 | 797.6141 | 536.8851 | 942.5659 | 899.6035 | 1535.679 | 1656.933 | 856.2068 | 2083.06 | 2453.683 |
| 23 | 1569.58 | 851.7427 | 1910.224 | 1494.121 | 1333.555 | 1411.226 | 1656.428 | 3068.977 | 1379.003 | 837.035 |
| 30 | 2437.906 | 1859.914 | 1211.515 | 734.4495 | 4021.896 | 1969.299 | 615.001 | 2093.644 | 2020.454 | 1399.598 |

### Example 4—In Vitro Expression-Effect of Multiple Doses

On Day 60 of the study described in Example 3 above, 5 out of the 10 mice from study Experiment No. 4 (Ab1 HC+LC 100 μg IV), Experiment No. 7 (Ab2 HC+LC 100 μg IV) and Experiment No. 9 (Ab1 SC 100 μg IM) received a second boost dose of the same cargo previously delivered (i.e., same vector, same route of administration, same dose, etc.). Time course of antibody levels for Ab1 HC+LC 100 μg IV (Exp. No. 4), Ab2 HC+LC 100 μg IV (Exp. No. 7), and Ab1 SC 100 μg IM (Exp. No. 9) formats from this experiment in single dose and redose formats (2nd dose received on day 60 of the study) is shown in FIG. 1G. The data provided in FIG. 1G was generated using a commercially available human IgG1 standard. Both IV formats displayed greatly enhanced IgG levels following boost compared to non-boost control, though no substantial effects were observed for the boost in the IM format. Surprisingly, for both IV re-doses, the effect of the second dose produced an antibody level that was greater than the expected additive effect.

### Example 5—High Dose Sub-Study

Additional groups of mice were added to the study described in Example 3 in order to ascertain the effect of higher doses administered in IM format (Experiment Nos. 15, 17, and 18 in Table 6 above). FIG. 1H shows time course antibody levels of single dose format for the Ab1 HC+LC 100 μg IV (Exp. No. 4), Ab1 HC+LC 250 μg IV (Exp. No. 17), Ab1 HC+LC 500 μg IV (Exp. No. 15), Ab1 T2A 30 μg IM (Exp. No. 10), Ab1 T2A 100 μg IM (Exp. No. 9), and Ab1 HC+LC 250 μg IM (Exp. No. 18) formats in mice. The data provided in FIG. 1H was generated using a commercially available human IgG1 standard. The results show a clear dose response and improved kinetic response of higher doses (i.e., faster rate of antibody generation). Antibody levels also remained relatively constant until the end point of the study (˜300 days or greater).

### Example 6—Commercial Standard IgG1 vs Internally Generated IgG1 Standard

All results reporting IgG or antibody concentrations above were made using the same commercially available human IgG1 standard (ThermoFisher IgG1 Human ELISA Standard (for Uncoated ELISA kit), cat. No. 39-50560-65). This commercially available standard was then compared against an internally generated IgG1 standard. The internal IgG1 standard was prepared according to the following protocol: Purified Ab1 and Ab2 proteins were produced by transient transfection of Expi293 cells (ThermoFisher). Heavy chain and light chain nanoplasmids were co-transfected at a ratio of 25 μg each plasmid into 50 ml suspension cell culture, using the manufacturer's recommended protocol. Supernatant was harvested on day 7 post-transfection and filter-sterilized with a 0.2 μm filter. IgG was purified from the supernatant using 2 ml Protein A resin (ThermoFisher). Supernatant was diluted with IgG binding buffer (ThermoFisher) before applying to resin. The resin was then washed with 5 column-volumes (CV) binding buffer, eluted with 2.5 CV IgG elution buffer (ThermoFisher), and neutralized with 1 M Tris, pH 8.0. Samples were buffer-exchanged into PBS. Sample concentration was measured using A280, purity was measured using SDS-PAGE, and functional activity was verified by antigen-binding ELISA.

FIG. 1J shows data from Exp. Nos. 15, 17, and 18 analyzed with the internally generated IgG1 standard (which contains overlapping samples with those shown in FIG. 1I measured with the commercial standard IgG1). The data indicates that antibody concentration values calculated with the internal standard are ˜25-fold lower than that of the commercial standard used in the experiments described above. This internal standard was used to calculate antibody concentrations in the experiments provided below, so this ˜25-fold correlation should be considered in comparisons between data generated by the two different standards (commercial vs. internal).

### Example 7—In Vivo Expression Testing Study 2

An additional in vivo mouse study to that described in Example 3 was carried out using the following experimental groups shown in Table 15 in order to further optimize in vivo antibody expression.

TABLE 15

| Experiment | | | | | | |
|---|---|---|---|---|---|---|
| Number | Payload | Dose | Route | Mice | n | Notes |
| 21 | Ab2 | 100 ug | IV | Rag2 | 8 | Positive |
| | HC + LC | | | | | Control |
| 22 | Ab2 | 100 ug | IV | B6 | 8 | |
| | HC + LC | | | | | |
| 23 | Ab2 | 100 ug | IV | Rag2 | 8 | 67.5 ug |
| | HC + LC | (1.7:1 | | | | HC + |
| | | HC:LC | | | | 32.5 ug |
| | | molar ratio) | | | | LC |
| 24 | Ab2 | 100 ug | IM | Rag2 | 8 | |
| | HC + LC | | | | | |
| 25 | Ab2 | 100 ug | IM | Rag2 | 8 | |
| | HC + LC | | | | | |
| 26 | Ab2 | 100 ug | IV | Rag2 | 8 | Liver |
| | HC + LC | | | | | formulation |

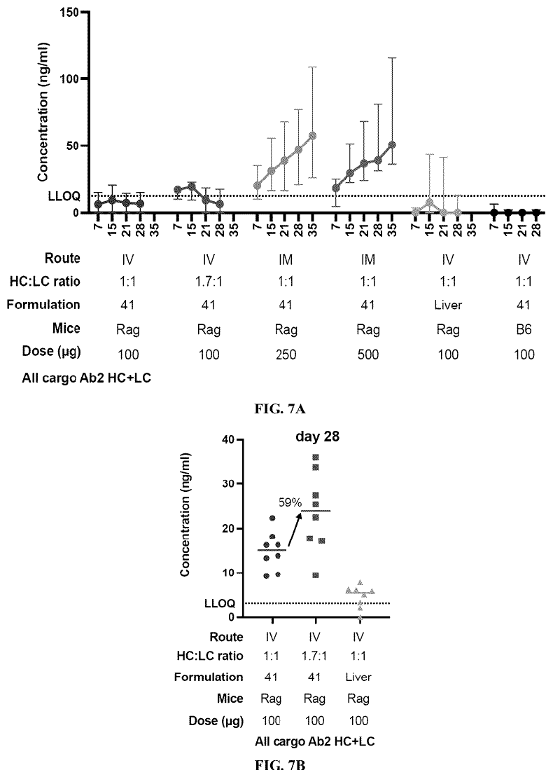

Blood samples were collected and processed to plasma every 7 days post-injection. Human IgG titers in mouse plasma were measured by electro-chemiluminescence assay (ECLIA) using a Meso Scale Discovery instrument. Human IgG titers in mice were quantified by measuring ECLIA signal of plasma samples diluted 1:25-1:100 and interpolated based on a standard curve of human IgG1 purified in-house, at concentrations ranging from 200 ng/ml-0.048 ng/ml. Standard curves were fit using nonlinear regression to a four-parameter sigmoidal dose-response curve. Results from initial time points of this experiment are shown in FIG. 7A. These results showed that the 250 microgram and 500 microgram IM doses behaved similarly, suggesting limited benefit in increasing dose beyond 250 micrograms. The liver formulation (high provided no apparent benefit over standard formulation.

Initial time points of experiments Exp. Nos. 21, 22, and 26 were below the lower limit of quantitation of the assay described above, though it is expected that the levels would rise in later time points. In order to better assess these earlier time points, a more sensitive assay was performed by coating the assay plate (Meso Scale Discovery) with the SARS-COV-2 Wuhan strain receptor binding domain (RBD) to enable better quantitation. The results of this experiment are shown in FIG. 7B. This experiment revealed a 60% increase in antibody levels at day 28 for the 1.7:1 HC: LC molar ratio group (Exp. No. 23) compared to the 1:1 HC: LC mas/mas ratio group (Exp. No. 21). It is expected that this trend will substantially continue at later time points as antibody levels continue to rise. This experiment also showed that the liver formulation performed worse than the standard formulation at this time point.

### Example 8—Assessment of SV40e Nuclear Localization Signal

Additional attempts to further raise the antibody level were attempted by using an SV50 enhancer (SV40e) into the Nanoplasmid expression vector (see, e.g., Hai-shan Li et al., “Enhancement of DNA Vaccine-Induced Immune Responses by a 72-Bp Element from SV40 Enhancer:,” Chinese Medical Journal 120, no. 6 (March 2007): 496-502, https://doi.org/10.1097/00029330-200703020-00012; i et al., “Muscle-Specific Enhancement of Gene Expression by Incorporation of SV40 Enhancer in the Expression Plasmid,” Gene Therapy 8, no. 6 (Mar. 1, 2001): 494-97, https://doi.org/10.1038/sj.gt.3301419; and Pontus Blomberg et al., “Electroporation in Combination with a Plasmid Vector Containing SV40 Enhancer Elements Results in Increased and Persistent Gene Expression in Mouse Muscle,” Biochemical and Biophysical Research Communications 298, no. 4 (November 2002): 505-10, https://doi.org/10.1016/S0006-291X (02) 02486-5). The sequence used in these experiments was TGGTTGCTGACTAATTGAGATGCATGCTTTGCATACTTCTGCCTGCTGGGGAGCC TGGGGACTTTCCACACC (SEQ ID NO: 102). This element is proposed to increase transgene expression in DNA gene therapy by providing a nuclear localization signal to target plasmid DNA to the nucleus of the cell. The SV40e element was constructed by incorporating the SV40e cassette directly upstream of the CAG promoter. This cassette was incorporated into both the heavy chain and light chain vectors in the split-vector configuration.

Initial attempts using the SV40e element in in vitro experiments in HEK293 cells did not show substantial difference in expression compared to those without SV40e. Plasmids incorporating the SV40e element were then administered in vivo to Rag 2 mice (e.g., as described in the Examples above) in the groups indicated in Table 16 below.

TABLE 16

| Experiment | | | | | |
|---|---|---|---|---|---|
| Number | Payload | Dose | Route | Mice | n |
| 31 | Ab1 WT HC + LC | 250 ug | IM | Rag2 | 5 |
| 32 | Ab1 SV40e HC + LC | 250 ug | IM | Rag2 | 5 |

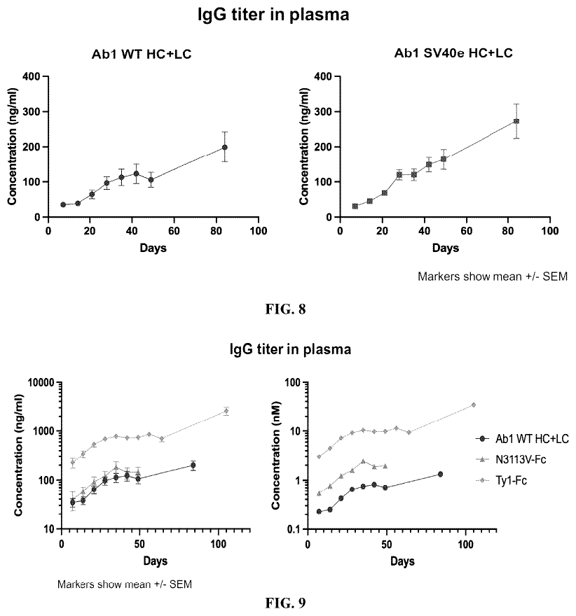

Results from this experiment are shown in FIG. 8. Results were measured using the inhouse generated IgG1 standard. Exp. No. 31 performed similarly to previous experiments testing the same payload at this dose. The SV40e vector showed ˜40% increase in expression overall. It is expected that this trend would continue at later time points and in other dose formats.

### Example 9—Assessment of V_{H}H-Fc Fusion Formats

Three V_{H}H-Fc fusion format antibodies derived from Camelid species were also tested (see Table 17 below). The V_{H}H fragments were fused to an Fc domain from human IgG1 (called V_{H}H-Fc) to increase neutralization potency and in vivo half-life. These constructs are N3113V-Fc and N3130V-Fc (both described in Li, et al 2022; doi 10.1016/j.cell.2022.03.009), and Ty1-Fc (described in Hanke, et al 2022; doi: 10.1038/s41467-020-18174-5). All three V_{H}H-Fc antibodies were designed using the following sequences in order: Kozak sequence; HC signal peptide; V_{H}H variable domain sequence; modified human hinge region; human CH2 and CH3 domains from IGHG1*01; and stop codon. The open reading frame was preceded by a CAG promoter and followed by a BGH poly-adenylation signal. All open reading frames were codon-optimized using commercially available software from ThermoFisher to reduce rare codon usage, balance GC content, and minimize RNA secondary structures.

TABLE 17

| | | | V_{H}H | | | |
|---|---|---|---|---|---|---|
| | | Signal | variable | Hinge | | |
| Name | Full Sequence | peptide | Domain | Region | CH2 | CH3 |
| N3113V- | MEFGLSWLFLVAILKGVQ | MEFG | EVQLVES | SDKT | PCPAPEL | GQPREP |
| Fc | C | L | GGGLVQP | HTCP | LGGPSVF | Q |
| | EVQLVESGGGLVQPGGSL | SWLFL | GGSLRLS | (SEQ | LFPPKPK | VYTLPPS |
| | R | VAILK | C | ID | DTLMISR | RDELTK |
| | LSCAASDSSFYDYEMSW | GVQC | AASDSSF | NO: | TPEVTCV | N |
| | VR | (SEQ | Y | 105) | VVDVSH | QVSLTCL |
| | QVPGKTPEWIGSMYPSGR | ID | DYEMSW | | EDPEVKF | VKGFYP |
| | T | NO: 111) | V | | NWYVD | S |
| | YINPSLKSLVTISRDNSEN | | RQVPGKT | | G | DIAVEWE |
| | M | | PEWIGSM | | VEVHNA | SNGQPE |
| | LYLQMNSLRAEDTAMYY | | YPSGRTYI | | KTKPREE | N |
| | C | | NPSLKSL | | QYNSTY | NYKTTP |
| | VSNWASGSTGDYWGQGT | | V | | R | P |
| | L | | TISRDNSE | | VVSVLTV | VLDSDG |
| | VTVSSSDKTHTCPPCPAPE | | NMLYLQ | | LHQDWL | S |
| | L | | M | | NGKEYK | FFLYSKL |
| | LGGPSVFLFPPKPKDTLMI | | NSLRAED | | CKVSNK | TVDKSR |
| | S | | TAMYYC | | ALPAPIE | WQQGN |
| | RTPEVTCVVVDVSHEDPE | | V | | KTISKAK | V |
| | V | | SNWASGS | | (SEQ ID | FSCSVM |
| | KFNWYVDGVEVHNAKT | | TGDYWG | | NO: 114) | H |
| | KP | | Q | | | EALHNH |
| | REEQYNSTYRVVSVLTVL | | GTLVTVS | | | YTQKSLSL |
| | H | | S | | | SPGK |
| | QDWLNGKEYKCKVSNK | | (SEQ ID | | | (SEQ ID |
| | AL | | NO: 112) | | | NO: 115) |
| | PAPIEKTISKAKGQPREPQ | | | | | |
| | V | | | | | |
| | YTLPPSRDELTKNQVSLTC | | | | | |
| | L | | | | | |
| | VKGFYPSDIAVEWESNGQ | | | | | |
| | P | | | | | |
| | ENNYKTTPPVLDSDGSFF | | | | | |
| | L | | | | | |
| | YSKLTVDKSRWQQGNVF | | | | | |
| | S | | | | | |
| | CSVMHEALHNHYTQKSL | | | | | |
| | SL | | | | | |
| | SPGK | | | | | |
| | (SEQ ID NO: 107) | | | | | |
| N3130V- | MEFGLSWLFLVAILKGVQ | MEFG | EVQLVES | SDKT | PCPAPEL | GQPREP |
| Fc | C | L | GGGLVQP | HTCP | LGGPSVF | Q |
| | EVQLVESGGGLVQPGGSL | SWLFL | GGSLRLS | (SEQ | LFPPKPK | VYTLPPS |
| | R | VAILK | C | ID | DTLMISR | RDELTK |
| | LSCAASDFYFDYYEMSW | GVQC | AASDFYF | NO: | TPEVTCV | N |
| | V | (SEQ | DYYEMS | 105) | VVDVSH | QVSLTCL |
| | RQAPGQGLEWVSTISGLG | ID | W | | EDPEVKF | VKGFYP |
| | G | NO: 111) | VRQAPGQ | | NWYVD | S |
| | ATYYADSVKGRFTISRDN | | GLEWVST | | G | DIAVEWE |
| | S | | I | | VEVHNA | SNGQPE |
| | KNTLYLQMNSLRAEDTA | | SGLGGAT | | KTKPREE | N |
| | L | | YYADSVK | | QYNSTY | NYKTTP |
| | YYCATRSPFGDYAFSYW | | GRFTISRD | | R | P |
| | G | | NSKNTLY | | VVSVLTV | VLDSDG |
| | QGTLVTVSSSDKTHTCPP | | LQMNSLR | | LHQDWL | S |
| | CP | | AEDTALY | | NGKEYK | FFLYSKL |
| | APELLGGPSVFLFPPKPKD | | YCATRSP | | CKVSNK | TVDKSR |
| | T | | F | | ALPAPIE | WQQGN |
| | LMISRTPEVTCVVVDVSH | | GDYAFSY | | KTISKAK | V |
| | E | | WGQGTL | | (SEQ ID | FSCSVM |
| | DPEVKFNWYVDGVEVHN | | V | | NO: 114) | H |
| | A | | TVSS | | | EALHNH |
| | KTKPREEQYNSTYRVVSV | | (SEQ | | | Y |
| | L | | ID NO: | | | TQKSLSL |
| | TVLHQDWLNGKEYKCKV | | 113) | | | SPGK |
| | S | | | | | (SEQ ID |
| | NKALPAPIEKTISKAKGQP | | | | | NO: 115) |
| | R | | | | | |
| | EPQVYTLPPSRDELTKNQ | | | | | |
| | V | | | | | |
| | SLTCLVKGFYPSDIAVEWE | | | | | |
| | S | | | | | |
| | NGQPENNYKTTPPVLDSD | | | | | |
| | G | | | | | |
| | SFFLYSKLTVDKSRWQQG | | | | | |
| | N | | | | | |
| | VFSCSVMHEALHNHYTQ | | | | | |
| | KS | | | | | |
| | LSLSPGK (SEQ ID NO: | | | | | |
| | 108) | | | | | |
| Ty 1-Fc | MEFGLSWLFLVAILKGVQ | MEFG | AQVQLVE | SDK | PCPAPEL | GQPREP |
| | C | L | TGGGLVQ | T | LGGPSVF | Q |
| | AQVQLVETGGGLVQPGGS | SWLF | PGGSLRL | HTCP | LFPPKPK | VYTLPPS |
| | L | L | S | (SEQ | DTLMISR | RDELTK |
| | RLSCAASGFTFSSVYMNW | VAILK | CAASGFT | ID | TPEVTCV | N |
| | V | GVQC | F | NO: | VVDVSH | QVSLTCL |
| | RQAPGKGPEWVSRISPNS | (SEQ | SSVYMN | 105) | EDPEVKF | VKGFYP |
| | G | ID | W | | NWYVD | S |
| | NIGYTDSVKGRFTISRDN | NO: 111) | VRQAPGK | | G | DIAVEW |
| | AK | | GPEWVSR | | VEVHNA | E |
| | NTLYLQMNNLKPEDTAL | | I | | KTKPREE | SNGQPE |
| | Y | | SPNSGNI | | QYNSTY | N |
| | YCAIGLNLSSSSVRGQGT | | G | | R | NYKTTP |
| | Q | | YTDSVKG | | VVSVLTV | P |
| | VTVSSSDKTHTCPPCPAPE | | RFTISRDN | | LHQDWL | VLDSDG |
| | L | | AKNTLYL | | NGKEYK | S |
| | LGGPSVFLFPPKPKDTLMI | | QMNNLK | | CKVSNK | FFLYSKL |
| | S | | P | | ALPAPIE | TVDKSR |
| | RTPEVTCVVVDVSHEDPE | | EDTALYY | | KTISKAK | WQQGN |
| | V | | CAIGLNL | | (SEQ ID | V |
| | KFNWYVDGVEVHNAKT | | S | | NO: 114) | FSCSVM |
| | KP | | SSSVRGQ | | | H |
| | REEQYNSTYRVVSVLTVL | | GTQVTVS | | | EALHNH |
| | H | | S (SEQ ID | | | Y |
| | QDWLNGKEYKCKVSNK | | NO: 97) | | | TQKSLSL |
| | AL | | | | | SPGK |
| | PAPIEKTISKAKGQPREPQ | | | | | (SEQ ID |
| | V | | | | | NO: 115) |
| | YTLPPSRDELTKNQVSLTC | | | | | |
| | L | | | | | |
| | VKGFYPSDIAVEWESNGQ | | | | | |
| | P | | | | | |
| | ENNYKTTPPVLDSDGSFF | | | | | |
| | L | | | | | |
| | YSKLTVDKSRWQQGNVF | | | | | |
| | S | | | | | |
| | CSVMHEALHNHYTQKSL | | | | | |
| | SL | | | | | |
| | SPGK | | | | | |
| | (SEQ ID NO: 109) | | | | | |

All three VHH-Fc constructs were found to express better than AB1 HC+LC format in vitro in HEK293 cells. Purified N3113V-Fc and N3130V-Fc were found to bind both SARSCOV-2 Wuhan and Omicron RBD with high affinity, whereas Ty-Fc did not substantial binding to Omicron RBD.

250 μg payloads of nanoplasmid vector encoding the VHH-Fc constructs (1 construct/vector) were administered via IM injection to three separate groups of Rag2 mice (n=4 or 5) similarly to the protocols described above in Example 3. Results from this experiment are shown in FIG. 9. N3130V-Fc did not yield any detectable level of antibody at any time point. Both N3113V-Fc and Ty1-Fc variants expressed better than Ab1 HC+LC format, with N3113V-Fc expressing ˜3-fold better than Ab1 on molar basis and Ty1-Fc expressing ˜10-15-fold better than Ab1 on molar basis.

### Example 10—WPRE Vector Assessment of VHH-Fc Fusion Format

The woodchuck hepatitis virus post-transcriptional regulatory element (WPRE) into the nanoplasmid expression vectors. This element has been previously reported to increase transgene expression in nonviral and viral vectors by improving transcription, stability, export, and translation of mRNA transcripts (e.g., Reinhard Klein et al., “WPRE-Mediated Enhancement of Gene Expression Is Promoter and Cell Line Specific,” Gene 372 (May 2006): 153-61, https://doi.org/10.1016/j.gene.2005.12.018; Lizheng Wang et al., “Enhancing Transgene Expression from Recombinant AAV8 Vectors in Different Tissues Using Woodchuck Hepatitis Virus Post-Transcriptional Regulatory Element,” International Journal of Medical Sciences 13, no. 4 (2016): 286-91, https://doi.org/10.7150/ijms. 14152) The WPRE vector was constructed by incorporating the WPRE cassette downstream of the antibody open reading frame, before BGH poly-adenylation signal. This cassette was incorporated into both the heavy chain and light chain vectors in the split-vector configuration, as well as into the Ty1-Fc VHH construct. When 100 μg payload of the Ty1-Fc VHH construct was administered to Rag2 mice as described above, the WPRE vector showed a ˜2-fold reduction in expression at day 7.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A system for expressing an antibody or an antigen binding fragment thereof in a subject, comprising:
a plasmid comprising a polynucleotide sequence encoding a heavy chain variable domain of the antibody or an antigen binding fragment thereof;
wherein the plasmid is encapsulated in a lipid vesicle; and
wherein when the plasmid encapsulated in the lipid vesicle is administered, the subject produces a peak blood plasma level of the antibody or antigen binding fragment thereof of at least 50 ng/mL.

2. A system for expressing an antibody or an antigen binding fragment thereof in a subject, comprising:
a plasmid comprising a polynucleotide sequence encoding a heavy chain variable domain of the antibody or an antigen binding fragment thereof;
wherein the plasmid is encapsulated in a lipid vesicle.

3. The system of claim 1 or 2, wherein the antibody or antigen binding fragment thereof is a single-domain antibody.

4. The system of claim any one of claims 1-3, wherein the antibody or antigen binding fragment thereof is a V_{H}H antibody.

5. The system of any one of claims 1-4, wherein the heavy chain variable domain is fused to an Fc domain, optionally through a peptide linker.

6. The system of claim any one of claims 1-3, wherein the plasmid encodes a full-length heavy chain of the antibody.

7. The system of any one of claims 1-6, wherein the plasmid further comprises a polynucleotide sequence encoding a light chain or an antigen binding fragment of the antibody.

8. The system of claim 7, wherein the plasmid encodes a full length light chain of the antibody.

9. The system of claim 7 or 8, wherein the polynucleotide sequence encoding the heavy chain variable domain and the polynucleotide sequence encoding the light chain are operably coupled such that the sequences are transcribed as a single transcript.

10. The system of claim 9, wherein the polynucleotide sequence encoding the heavy chain and the polynucleotides sequence encoding the light chain are separated by a self-cleavage peptide encoding sequence.

11. The system of any one of claim 1, 2, or 6, further comprising a second plasmid comprising a second polynucleotide sequence encoding a light chain of the antibody.

12. The system of claim 11, wherein the plasmid and the second plasmid are present in a ratio of about 1.7:1.

13. The system of claim 11 or 12, wherein the light chain of the antibody is a kappa chain or a lambda chain.

14. The system of any one of claims 11-13, wherein the second plasmid is also encapsulated in a lipid vesicle.

15. The system of any one of claims 1-14, wherein the lipid vesicle comprises a fusion-associated small transmembrane (FAST) protein.

16. The system of claim 15, wherein the FAST protein comprises domains from one or more FAST proteins selected from p10, p14, p15, and p22.

17. The system of claim 15 or 16, wherein the FAST protein comprises an amino acid sequence having at least 80% sequence identity to the sequence:
(SEQ ID NO: 203)
MGSGPSNFVNHAPGEAIVTGLEKGADKVAGTISHTIFVEIVSSSTGIII
AVGIFAFIFSFL
YKLLQWYNRKSKNKKRKEQIREQIELGLLSYGAGVASLPLLNVIAHNPG
SVISATPIY
KGPCTGVPNSRLLQITSGTAEENTRILNHDGRNPDGSINV

18. The system of any one of the preceding claims, wherein the plasmid comprises a promoter operably linked to the polynucleotide sequence selected from CAG, CMV, EFIA, CBh, CBA, and SFFV.

19. The system of claim 18, wherein the plasmid comprises the CAG promoter.

20. The system of any one of the preceding claims, wherein the antibody or antigen binding fragment thereof comprises an IgG1, IgG2a, IgG2b, IgG3, IgG4, IgD, IgM, IgA1, IgA2 or IgE heavy chain.

21. The system of claim 20, wherein the antibody or antigen binding fragment thereof comprises the IgG1, IgG2a, IgG2b, IgG3, or IgG4 heavy chain.

22. The system of claim 20, wherein the antibody comprises the IgG1 heavy chain.

23. The system of any one of the preceding claims, wherein the heavy chain variable domain comprises a sequence that is at least 80% sequence identity to
(SEQ ID NO: 101)
AQVQLVETGGGLVQPGGSLRLSCAASXXXXXXXXXMNWVRQAPGKGPEW
VSXXXXXXX
XXXYTDSVKGRFTISRDNAKNTLYLQMNNLKPEDTALYYCXXXXXXX
XXXXRGQGTQVTVSS, wherein each X is independently
absent or any amino acid.

24. The system of any one of the preceding claims, wherein the antibody or antigen binding fragment comprises an Fc domain having one or more mutations or combinations of mutations selected from Arg435His (His435), Asn434Ala (A), Met428Leu/Asn434Ser (LS), Thr252Leu/Thr253Ser/Thr254Phe (LSF), Glu294delta/Thr307Pro/Asn434Tyr (C6A-66), Thr256Asn/Ala378Val/Ser383Asn/Asn434Tyr (C6A-78), and Glu294delta (Del), wherein residue position number is based on EU numbering convention.

25. The system of any one of the preceding claims, wherein the antibody or antigen binding fragment thereof comprises an Fc domain having one or more mutations selected from M252Y, S254T, T256E, and any combination thereof, wherein residue position numbering is based on EU numbering convention.

26. The system of any one of the preceding claims, wherein the plasmid comprises a SV40e element.

27. The system of any one of the preceding claims, wherein the antibody or antigen binding fragment thereof binds specifically to a viral protein.

28. The system of claim 27, wherein the viral protein from a virus selected from a group consisting of a parvovirus, a picornavirus, a rhabdovirus, a paramyxovirus, an orthomyxovirus, a bunyavirus, a calicivirus, an arenavirus, a polyomavirus, a reovirus, a togavirus, a bunyavirus, a herpes simplex virus, a poxvirus, an adenovirus, a coxsackievirus, a flavivirus, a coronavirus, an astrovirus, an enterovirus, a rotavirus, a norovirus, a retrovirus, a papilloma virus, a parvovirus, an influenza virus, a hemorrhagic fever virus, and a rhinovirus.

29. The system of claim 28, wherein the viral protein is from a virus select from a group consisting of Hantavirus, Rabies, Nipah, Hendra, Rift Valley Fever, Lassa, Marburg, Crimean Congo Fever, hMPV, RSV, Hepatitis A, Hepatitis B, Hepatitis C, Hepatitis D, Hepatitis E, Norovirus, Monkeypox, Coxpox, Japanese Encephalitis, Yellow Fever, HSV-1, HSV-2, MERS, ChickenPox, Hand, Foot and Mouth, CMV (HHV-5), Equine Encephalitis, EBV (HHV-4), Human Metapneumo virus, Norovirus, Enterovirus, Smallpox, West Nile Virus, Paramyxoviridae, Rhino virus, Mononucleosis, coxsackievirus B, Influenza, polio, Measles, Rubella, HPV, Zika, Mumps, Herpes viridae, Chikungunya, H. influenzae, and SARS-COV2 viruses.

30. The system of claim 27, wherein the viral protein is from SARS-COV-2.

31. The system of claim 30, wherein the viral protein is a SARS-COV-2 spike protein.

32. The system of any one of the preceding claims, wherein the antibody or antigen binding fragment thereof comprises an amino acid sequence having at least 80%, at least 85%, at least 90%, at least 95%, or at least 98% sequence identity to an antibody set forth in Table 3.

33. The system of any one of claims 1-26, wherein the antibody or antigen binding fragment binds specifically to a cancer antigen.

34. The system of claim 33, wherein the cancer antigen is selected from the group consisting of programmed cell death 1 (PD1) programmed cell death ligand 1 (PDL1), CD5, CD20, CD19, CD22, CD30, CD33, CD40, CD44, CD52, CD74, CD103, CD137, CD123, CD152, a carcinoembryonic antigen (CEA), an integrin, an epidermal growth factor (EGF) receptor family member, a vascular epidermal growth factor (VEGF), a proteoglycan, a disialoganglioside, B7-H3, cancer antigen 125 (CA-125), epithelial cell adhesion molecule (EpCAM), vascular endothelial growth factor receptor 1, vascular endothelial growth factor receptor 2, a tumor associated glycoprotein, mucin 1 (MUC1), a tumor necrosis factor receptor, an insulin-like growth factor receptor, folate receptor a, transmembrane glycoprotein NMB, a C-C chemokine receptor, prostate specific membrane antigen (PSMA), recepteur d'origine nantais (RON) receptor, cytotoxic T-lymphocyte antigen 4 (CTLA4), Colon cancer antigen 19.9, gastric cancer mucin antigen 4.2, colorectal carcinoma antigen A33, ADAM-9, AFP oncofetal antigen-alpha-fetoprotein, ALCAM, BAGE, beta-catenin, Carboxypeptidase M, B1, CD23, CD25, CD27, CD28, CD36, CD45, CD46, CD52, CD56,CD79a/CD79b, CD317, CDK4, CO-43 (blood group Leb), CO-514 (blood group Lea), CTLA-1, Cytokeratin 8, DR5, El series (blood group B), Ephrin receptor A2 (EphA2), Erb (ErbB1, ErbB3, ErbB4), lung adenocarcinoma antigen F3, antigen FC10.2, GAGE-1, GAGE-2, GD2/GD3/GD49/GM2/GM3, GICA 19-9, gp37, gp75, gp100, HER-2/neu, human milk fat globule antigen, human papillomavirus-E6/human papillomavirus-E7, high molecular weight melanoma antigen (HMW-MAA), differentiation antigen (I antigen), I (Ma) as found in gastric adenocarcinomas, Integrin Alpha-V-Beta-6, Integrinβ6 (ITGB6), Interleukin-13 Receptor a2 (IL13Ra2), JAM-3, KID3, KID31, KS 1/4 pan-carcinoma antigen, KSA (17-1A), human lung carcinoma antigen L6, human lung carcinoma antigen L20, LEA, LUCA-2, M1: 22:25:8, M18, M39, MAGE-1, MAGE-3, MART, Myl, MUM-1, N-acetylglucosaminyltransferase, neoglycoprotein, NS-10, OFA-1 and OFA-2, Oncostatin M (Oncostatin Receptor Beta), rho15, prostate specific antigen (PSA), PSMA, polymorphic epithelial mucin antigen (PEMA), PIPA, prostatic acid phosphate, R24, ROR1, SSEA-1, SSEA-3, SSEA-4, sTn, T cell receptor derived peptide, T5A7, Tissue Antigen 37, TAG-72, TL5 (blood group A), a TNF-α receptor (TNFαR), TNFβR, TNFγR, TRA-1-85 (blood group H), Transferrin Receptor, TSTA tumor-specific transplantation antigen, VEGF-R, Y hapten, Ley, and 5T4.

35. The system of any one of claims 1-26, wherein the antibody or antigen binding fragment thereof binds specifically to a protein or component of a bacteria.

36. The system of claim 35, wherein the bacteria is Bacillus anthracis, Corynebacterium diphtheria, Bordetella pertussis, Streptococcus pneumonia, Haemophilus influenza, Salmonella typhimurium, a Shigella species, a Streptococcus species, Chlamydia trachomatis, Yersinia pestis, Methicillin-resistant Staphylococcus aureus (MRSA), Staphylococcus aureus, Clostridium tetani, Vibrio cholera, Escherichia coli, Klebsiella pneumonia, Borrelia burgdorferi, Borrelia mayonii, Clostridioides difficile, Pseudomonas aeruginosa, Helicobacter pylori, Streptococcus pyogenes, Francisella tularensis, an Acinetobacter species, Neisseria gonorrhoeae, a Leptospira species, Coxiella burnetii, Clostridium botulinum, Burkholderia pseudomallei, a gram-negative bacteria, Salmonella paratyphi, Mycobacterium leprae, a Brucella species, a Campylobacter species, Listeria monocytogenes, Mycobacterium avium, Mycoplasma pneumonia, a Rickettsia species, a Anaplasma species, an Ehrlichia species, a Neorickettsia species, a Neoehrlichia species, a Orientia species, Mycobacterium tuberculosis, Anaplasam phagocytophilum, Orientia tsutsugamushi, or a Bartonella species.

37. The system of any one of claims 1-26, wherein the antibody or antigen binding fragment thereof binds specifically to a protein or component of a parasite.

38. The system of claim 37, wherein the parasite is a Babesia species, Ancylostoma duodenale, Necator americanus, Sarcoptes scabiei, Ascaris lumbricoides, Schistosoma mansoni, Taenia solium, Enterobius vermicularis, Wuchereria bancrofti, Toxoplasma gondii, Giardia lamblia, Entamoeba histolytica, a Plasmodium species, a Leishmania species, Trypanosoma cruzi, a Schistosoma species, a Cryptosporidium species, Trypanosoma brucei, Wuchereria bancrofti, Brugia malayi, Brugia timori, Entamoeba histolytica, or Onchocerca volvulus.

39. The system of any one of claims 1-26, wherein the antibody or antigen binding fragment thereof binds specifically to an allergen.

40. The system of claim 39, wherein the allergen is derived from a mite, an insect, a pollen, an animal epithelium, a mold, meat, a fish, a crustacean, a fruit, a nut, a vegetable, a flour or bran, a milk, an egg, a spice, hay, silk, cotton, latex, a yeast, a grass, a tree, a cereal, or an animal hair.

41. The system of any one of claims 1-26, wherein the antibody or antigen binding fragment thereof binds specifically to an immune checkpoint molecule.

42. The system of claim 41, wherein the immune checkpoint molecule is PD-1, PD-L1, CTLA-4, TIM-3, TIGIT, 4-1BB (CD137), GITR (CD357), or a killer IgG-like receptor (KIR).

43. The system of any one of claims 1-26, wherein the antibody or antigen binding fragment thereof binds specifically to an antigen implicated in an inflammatory disease.

44. The system of claim 43, wherein the inflammatory disease selected from allergy, asthma, coeliac disease, glomerulonephritis, hepatitis, and inflammatory bowel disease.

45. The system of claim 43, wherein the inflammatory disease is Mast Cell Activation Syndrome (MCAS).

46. The system of claim 43, wherein the inflammatory disease is an autoimmune disease selected from rheumatoid arthritis, psoriasis, Guillain-Barre syndrome, Graves' disease, Mysathenia gravis, vasculitis, lupus, Type 1 diabetes, Hashimoto's disease, inflammatory bowel disease, Celiac disease, or multiple sclerosis (MS).

47. The system of claim 43, wherein the inflammatory disease is an autoinflammatory disease selected from Familial Mediterranean fever (FMF), Cryopyrin-associated periodic syndromes (CAPS), TNF receptor-associated periodic syndrome (TRAPS), Deficiency of IL-1-receptor antagonist (DIRA), or Hyper IgD syndrome (HIDS).

48. The system of any one of the preceding claims, the administering produces a peak blood plasma level of the antibody or antigen binding fragment thereof of at least 75 ng/mL, at least 100 ng/mL, at least 150 ng/ml, at least 200 ng/mL, at least 250 ng/ml, at least 300 ng/mL, at least 400 ng/mL, at least 500 ng/mL, at least 600 ng/ml, at least 700 ng/ml, at least 800 ng/mL, at least 900 ng/mL, or at least 1000 ng/mL.

49. The system of any one of the preceding claims, wherein the administering occurs without electroporation or hydroporation.

50. The system of any one of the preceding claims, wherein the plasmid is a DNA plasmid.

51. A method of inducing antibody production in the subject, comprising administering to the subject the system of any one of the preceding claims.

52. A method of inducing antibody production in a subject, comprising administering to the subject:
a plasmid comprising a polynucleotide sequence encoding a heavy chain variable domain of the antibody or an antigen binding fragment thereof;
wherein the plasmid is encapsulated in a lipid vesicle; and
wherein administration of the plasmid encapsulated in the lipid vesicle to the subject produces a blood plasma level of the antibody or antigen binding fragment thereof of at least 50 ng/mL.

53. The method of claim 51 or 52, wherein the administering is performed intramuscularly, subcutaneously, intradermally, intranasally, orally, intrathecally, or intravenously.

54. The method of any one of claims 51-53, wherein the administering is performed intramuscularly.

55. The method of any one of claims 51-53, wherein the administering is performed intravenously.

56. The method of any one of claims 51-55, wherein the administering is performed without electroporation or hydroporation.

57. The method of any one of claims 51-56, wherein the administering produces a peak blood plasma level of the antibody or antigen binding fragment thereof of at least 75 ng/ml, at least 100 ng/ml, at least 150 ng/ml, at least 200 ng/mL, at least 250 ng/ml, at least 300 ng/mL, at least 400 ng/mL, at least 500 ng/mL, at least 600 ng/ml, at least 700 ng/ml, at least 800 ng/ml, at least 900 ng/ml, or at least 1000 ng/mL.

58. The method of any one of claims 51-57, wherein the administering occurs 1 or 2 times.

59. The method of any one of claims 51-57, wherein the method comprises administering 2 doses of the plasmid to the subject.

60. The method of claim 59, wherein the 2 doses are administered from about 2 weeks to about 12 weeks apart.

61. The method of claim 59 or 60, wherein administration of the second dose results in peak blood plasma level of the antibody or antigen binding fragment which is greater than 2fold higher than the peak blood plasma level achieved after the first dose.

62. The method of claim 59 or 60, wherein administration of the second dose results in a peak blood plasma level of the antibody or antigen binding fragment which is at least 3-fold, at least 4-fold, or at least 5-fold higher than the peak blood plasma level achieved after the first dose.

63. The method of any one of claims 51-62, wherein the administering comprises delivery of from about 0.1 mg/kg to about 10 mg/kg of the plasmid to the subject per dose.

64. The method of any one of claims 51-63, wherein the blood plasma level of the antibody or antigen binding fragment is sustained at a concentration of at least 50 ng/mL, at least 100 ng/mL, at least 200 ng/mL, at least 300 ng/ml, at least 400 ng/ml, at least 500 ng/mL, at least 500 ng/mL, at least 600 ng/mL, at least 700 ng/ml, at least 800 ng/mL, at least 900 ng/ml, or at least 1000 ng/ml for a period of at least 4 weeks, at least 6 weeks, at least 8 weeks, at least 10 weeks, or at least 20 weeks after the administration.

65. The method of any one of claims 51-64, wherein the blood plasma level of the antibody or antigen binding fragment is sustained at a concentration of at least 50% of the peak blood plasma concentration achieved for a period of at least 4 weeks, at least 6 weeks, at least 8 weeks, at least 10 weeks, at least 20 weeks, at least 30 weeks, or at least 40 weeks after the administration.

66. The method of any one of claims 51-64, wherein the blood plasma level of the antibody or antigen binding fragment is sustained at a concentration of at least 25% of the peak blood plasma concentration achieved for a period of at least 4 weeks, at least 6 weeks, at least 8 weeks, at least 10 weeks, at least 20 weeks, at least 30 weeks, or at least 40 weeks after the administration.

67. The method of any one of claims 51-64, wherein the blood plasma level of the antibody or antigen binding fragment is sustained at a concentration of at least 10% of the peak blood plasma concentration achieved for a period of at least 4 weeks, at least 6 weeks, at least 8 weeks, at least 10 weeks, at least 20 weeks, at least 30 weeks, or at least 40 weeks after the administration.

68. The method of any one of claims 65-67, wherein the sustained concentration of antibody is achieved after a single administration.

69. The method of any one of claims 65-67, wherein the sustained concentration of the antibody or antigen binding fragment is achieved after two administrations.


SEQUENCE LISTING (208 sequences)

SEQ ID NO: 1 - protein, 120 aa, synthetic construct
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSS

SEQ ID NO: 2 - protein, 8 aa, synthetic construct
GFTFSDYY

SEQ ID NO: 3 - protein, 8 aa, synthetic construct
ITYSGSTI

SEQ ID NO: 4 - protein, 13 aa, synthetic construct
ARDRGTTMVPFDY

SEQ ID NO: 5 - protein, 107 aa, synthetic construct
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIK

SEQ ID NO: 6 - protein, 6 aa, synthetic construct
QDITNY

SEQ ID NO: 7 - skipped

SEQ ID NO: 8 - protein, 9 aa, synthetic construct
QQYDNLPLT

SEQ ID NO: 9 - protein, 120 aa, synthetic construct
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSS

SEQ ID NO: 10 - protein, 8 aa, synthetic construct
GFTFSNYA

SEQ ID NO: 11 - protein, 8 aa, synthetic construct
ISYDGSNK

SEQ ID NO: 12 - protein, 13 aa, synthetic construct
ASGSDYGDYLLVY

SEQ ID NO: 13 - protein, 110 aa, synthetic construct
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVL

SEQ ID NO: 14 - protein, 9 aa, synthetic construct
SSDVGGYNY

SEQ ID NO: 15 - skipped

SEQ ID NO: 16 - protein, 10 aa, synthetic construct
NSLTSISTWV

SEQ ID NO: 17 - protein, 123 aa, synthetic construct
QMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSS

SEQ ID NO: 18 - protein, 8 aa, synthetic construct
GFTFMSSA

SEQ ID NO: 19 - protein, 8 aa, synthetic construct
IVIGSGNT

SEQ ID NO: 20 - protein, 16 aa, synthetic construct
AAPYCSSISCNDGFDI

SEQ ID NO: 21 - protein, 99 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWT

SEQ ID NO: 22 - protein, 8 aa, synthetic construct
SQSVSSSY

SEQ ID NO: 23 - skipped

SEQ ID NO: 24 - protein, 10 aa, synthetic construct
QHYGSSRGWT

SEQ ID NO: 25 - protein, 119 aa, synthetic construct
EVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSS

SEQ ID NO: 26 - protein, 8 aa, synthetic construct
GFTFRDVW

SEQ ID NO: 27 - protein, 10 aa, synthetic construct
IKSKIDGGTT

SEQ ID NO: 28 - protein, 22 aa, synthetic construct
TTAGSYYYDTVGPGLPEGKFDY

SEQ ID NO: 29 - protein, 102 aa, synthetic construct
DIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLT

SEQ ID NO: 30 - protein, 12 aa, synthetic construct
QSVLYSSNNKNY

SEQ ID NO: 31 - skipped

SEQ ID NO: 32 - protein, 8 aa, synthetic construct
QQYYSTLT

SEQ ID NO: 33 - protein, 119 aa, synthetic construct
QVQLVESGGGVVQPGRSLRLSCAASGFTFSSYAMHWVRQAPGKGLEWVAVIPFDGRNKYYADSVTGRFTISRDNSKNTLYLQMNSLRAEDTAVYYCASSSGYLFHSDYWGQGTLVTVSS

SEQ ID NO: 34 - protein, 8 aa, synthetic construct
GFTFSSYA

SEQ ID NO: 35 - protein, 8 aa, synthetic construct
IPFDGRNK

SEQ ID NO: 36 - protein, 12 aa, synthetic construct
ASSSGYLFHSDY

SEQ ID NO: 37 - protein, 107 aa, synthetic construct
DIQMTQSPSTLSASVGDRVTITCRASQSISNWLAWFQQKPGKAPKLLIYEASSLESGVPSRFSGSGSGTEFTLTISSLQPDDFATYYCQQYNSYPWTFGQGTKVEIK

SEQ ID NO: 38 - protein, 6 aa, synthetic construct
QSISNW

SEQ ID NO: 39 - skipped

SEQ ID NO: 40 - protein, 9 aa, synthetic construct
QQYNSYPWT

SEQ ID NO: 41 - protein, 131 aa, synthetic construct
EVQLVESGGGLIQPGGSLRLSCAASGFTVSNNYMSWVRQAPGKGLEWVSVIYSGGSTYYADSVKGRFTISRDKSKNTLYLQMNRLRAEDTAVYYCAREGEVEGYNDFWSGYSRDRYYFDYWGQGTLVTVSS

SEQ ID NO: 42 - protein, 8 aa, synthetic construct
GFTVSNNY

SEQ ID NO: 43 - protein, 7 aa, synthetic construct
IYSGGST

SEQ ID NO: 44 - protein, 25 aa, synthetic construct
AREGEVEGYNDFWSGYSRDRYYFDY

SEQ ID NO: 45 - protein, 110 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 46 - protein, 9 aa, synthetic construct
SSDVGGYNY

SEQ ID NO: 47 - skipped

SEQ ID NO: 48 - protein, 10 aa, synthetic construct
SSYTSSSTRV

SEQ ID NO: 49 - protein, 113 aa, synthetic construct
EVQLVESGGGLVKPGGSLRLSCAASGFTFSSYYMNWVRQAPGKGLEWVSSISEDGYSTYYPDSLKGRFTISRDSAKNSLYLQMNSLRADDTAVYYCARDFSGHTAWAGTGFEY

SEQ ID NO: 50 - protein, 8 aa, synthetic construct
GFTFSSYY

SEQ ID NO: 51 - protein, 8 aa, synthetic construct
ISEDGYST

SEQ ID NO: 52 - protein, 17 aa, synthetic construct
ARDFSGHTAWAGTGFEY

SEQ ID NO: 53 - protein, 102 aa, synthetic construct
QSVLTQPPSVSGAPGQRITISCTGSSSNIGAGYDVHWYQQLPGTAPKLLIYGSSSRNSGVPDRFSGSKSGTSASLAITGLQAEDEADYYCQSYDSSLSVLYT

SEQ ID NO: 54 - protein, 8 aa, synthetic construct
SSNIGAGY

SEQ ID NO: 55 - skipped

SEQ ID NO: 56 - protein, 12 aa, synthetic construct
QSYDSSLSVLYT

SEQ ID NO: 57 - protein, 125 aa, synthetic construct
QVQLVQSGAEVKKPGSSVKVSCKASGGTFSSYTISWVRQAPGQGLEWMGRIIPILDRVMYAQKFQGRVTITADKSTSTAYMELSSLRSEDTAVYYCARRAIDSDTYVEQSHFDYWGQGTLVTVSS

SEQ ID NO: 58 - protein, 8 aa, synthetic construct
GGTFSSYT

SEQ ID NO: 59 - protein, 8 aa, synthetic construct
IIPILDRV

SEQ ID NO: 60 - protein, 18 aa, synthetic construct
ARRAIDSDTYVEQSHFDY

SEQ ID NO: 61 - protein, 103 aa, synthetic construct
EIVMTQSPATLSLSPGERATLSCRASQSVSSYLAWYQQKPGQAPSLLIYDASNRATGIPARFSGSGSGTDFTLTISSLEPEDFAVYYCQQPLTFGGGTKVEIK

SEQ ID NO: 62 - protein, 6 aa, synthetic construct
QSVSSY

SEQ ID NO: 63 - skipped

SEQ ID NO: 64 - protein, 5 aa, synthetic construct
QQPLT

SEQ ID NO: 65 - protein, 108 aa, synthetic construct
QVQLVQSGSELKKPGASVKVSCKASGYTFTTYVMNWVRQAPGQGLEWMGWINTNTGNPTYAQGFTGRFVFSLDTSVSTASLQISSLKAEDTAVYYCSSEITTLGGMDV

SEQ ID NO: 66 - protein, 8 aa, synthetic construct
GYTFTTYV

SEQ ID NO: 67 - protein, 8 aa, synthetic construct
INTNTGNP

SEQ ID NO: 68 - protein, 12 aa, synthetic construct
SSEITTLGGMDV

SEQ ID NO: 69 - protein, 108 aa, synthetic construct
SYVLTQPPSVSVAPGKTARITCGGNNIGSKSVHWYQQKPGQAPVLVIYYDSDRPSGIPERFSGSNSGNTATLTISGVEAGDEADYYCQVWDSISDHRVFGGGTKLTVL

SEQ ID NO: 70 - protein, 6 aa, synthetic construct
NIGSKS

SEQ ID NO: 71 - skipped

SEQ ID NO: 72 - protein, 11 aa, synthetic construct
QVWDSISDHRV

SEQ ID NO: 73 - protein, 117 aa, synthetic construct
EVQLVESGGGLVQPGGSLRLSCAASGITVSSNYMNWVRQAPGKGLEWVSLIYSGGSTYYADSVKGRFTISRDNSKNTLYLQMNSLRAEDTAVYHCARDLVVYGMDVWGQGTTVTVSS

SEQ ID NO: 74 - protein, 8 aa, synthetic construct
GITVSSNY

SEQ ID NO: 75 - protein, 7 aa, synthetic construct
IYSGGST

SEQ ID NO: 76 - protein, 11 aa, synthetic construct
ARDLVVYGMDV

SEQ ID NO: 77 - protein, 107 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPTFGQGTKLEIK

SEQ ID NO: 78 - protein, 8 aa, synthetic construct
SQSVSSSY

SEQ ID NO: 79 - skipped

SEQ ID NO: 80 - protein, 8 aa, synthetic construct
QQYGSSPT

SEQ ID NO: 81 - protein, 118 aa, synthetic construct
EVQLVESGGGLIQPGGSLRLSCAASGFIVSSNYMSWVRQAPGKGLEWVSIIYSGGSTFYADSVKGRFTISRDNSKNTLYLQMNSLRVEDTAVYYCARDLQELGSLDYWGQGTLVTVSS

SEQ ID NO: 82 - protein, 8 aa, synthetic construct
GFIVSSNY

SEQ ID NO: 83 - protein, 7 aa, synthetic construct
IYSGGST

SEQ ID NO: 84 - protein, 12 aa, synthetic construct
ARDLQELGSLDY

SEQ ID NO: 85 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQEANSFPYTFGQGTKLEIK

SEQ ID NO: 86 - protein, 7 aa, synthetic construct
QGISSWL

SEQ ID NO: 87 - skipped

SEQ ID NO: 88 - protein, 9 aa, synthetic construct
QEANSFPYT

SEQ ID NO: 89 - protein, 120 aa, synthetic construct
QVQLVESGGGVVQPGRSLRLSCAATGFTFRRYGMHWVRQAPGKGLEWVAGILFDGSNKYYVDSVKGRFTISRDSSRNTLYLQLNSLRREDTAVYYCAKGGDYEWELLESWGQGTLVTVSS

SEQ ID NO: 90 - protein, 8 aa, synthetic construct
GFTFRRYG

SEQ ID NO: 91 - protein, 8 aa, synthetic construct
ILFDGSNK

SEQ ID NO: 92 - protein, 13 aa, synthetic construct
AKGGDYEWELLES

SEQ ID NO: 93 - protein, 107 aa, synthetic construct
DIQMTQSPSTVSASVGDRVTITCRASQSIDNWLAWYQEKPGKAPKVLIYKASSLESGVPSRFSGRGSGTEFTLTISSLQPGDFATYYCQHYHSFPLTFGGGTKVDIK

SEQ ID NO: 94 - protein, 7 aa, synthetic construct
QSIDNWL

SEQ ID NO: 95 - skipped

SEQ ID NO: 96 - protein, 9 aa, synthetic construct
QHYHSFPLT

SEQ ID NO: 97 - protein, 119 aa, synthetic construct
AQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSS

SEQ ID NO: 98 - protein, 9 aa, synthetic construct
GFTFSSVYM

SEQ ID NO: 99 - protein, 10 aa, synthetic construct
RISPNSGNIG

SEQ ID NO: 100 - protein, 11 aa, synthetic construct
AIGLNLSSSSV

SEQ ID NO: 101 - protein, 120 aa, synthetic construct
  VARIANT 27..35: note = X can be any amino acid
  VARIANT 54..61: note = X can be any amino acid
  VARIANT 99..109: note = X can be any amino acid
  REGION 27..35: note = Up to 9 "X" residues may be deleted
  REGION 54..61: note = Up to 10 "X" residues may be deleted
  REGION 99..109: note = Up to 11 "X" residues may be deleted
AQVQLVETGGGLVQPGGSLRLSCAASXXXXXXXXXMNWVRQAPGKGPEWVSXXXXXXXXXXYTDSVKGRFTISRDNAKNTLYLQMNNLKPEDTALYYCXXXXXXXXXXXRGQGTQVTVSS

SEQ ID NO: 102 - DNA, 72 bp, synthetic construct
  source 1..72: mol_type = other DNA
tggttgctgactaattgagatgcatgctttgcatacttctgcctgctggggagcctggggactttccacacc

SEQ ID NO: 103 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 104 - protein, 25 aa, synthetic construct
RRKRGSGEGRGSLLTCGDVEENPGP

SEQ ID NO: 105 - protein, 8 aa, synthetic construct
SDKTHTCP

SEQ ID NO: 106 - protein, 220 aa, synthetic construct
PCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 365 aa, synthetic construct
MEFGLSWLFLVAILKGVQCEVQLVESGGGLVQPGGSLRLSCAASDSSFYDYEMSWVRQVPGKTPEWIGSMYPSGRTYINPSLKSLVTISRDNSENMLYLQMNSLRAEDTAMYYCVSNWASGSTGDYWGQGTLVTVSSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 367 aa, synthetic construct
MEFGLSWLFLVAILKGVQCEVQLVESGGGLVQPGGSLRLSCAASDFYFDYYEMSWVRQAPGQGLEWVSTISGLGGATYYADSVKGRFTISRDNSKNTLYLQMNSLRAEDTALYYCATRSPFGDYAFSYWGQGTLVTVSSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 366 aa, synthetic construct
MEFGLSWLFLVAILKGVQCAQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - skipped

SEQ ID NO: 111 - protein, 19 aa, synthetic construct
MEFGLSWLFLVAILKGVQC

SEQ ID NO: 112 - protein, 118 aa, synthetic construct
EVQLVESGGGLVQPGGSLRLSCAASDSSFYDYEMSWVRQVPGKTPEWIGSMYPSGRTYINPSLKSLVTISRDNSENMLYLQMNSLRAEDTAMYYCVSNWASGSTGDYWGQGTLVTVSS

SEQ ID NO: 113 - protein, 120 aa, synthetic construct
EVQLVESGGGLVQPGGSLRLSCAASDFYFDYYEMSWVRQAPGQGLEWVSTISGLGGATYYADSVKGRFTISRDNSKNTLYLQMNSLRAEDTALYYCATRSPFGDYAFSYWGQGTLVTVSS

SEQ ID NO: 114 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 115 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116-199 - skipped (84 entries)

SEQ ID NO: 200 - protein, 1272 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 201 - protein, 154 aa, synthetic construct
MGSGPSNFVNHAPGEAIVTGLEKGADKVAGTISHTIWEVIAGLVALLTFLAFGFWLFKYLQWYNRKSKNKKRKEQIREQIELGLLSYGAGVASLPLLNVIAHNPGSVISATPIYKGPCTGVPNSRLLQITSGTAEENTRILNHDGRNPDGSINV

SEQ ID NO: 202 - protein, 125 aa, synthetic construct
MGSGPSNFVNHAPGEAIVTGLEKGADKVAGTISHTIWEVIAGLVALLTFLAFGFWLFKYLQKRRERRRQLTEFQKRYLRNSYRLSEIQRPISQHEYEDPYEPPSRRKPPPPPYSTYVNIDNVSAI

SEQ ID NO: 203 - protein, 159 aa, synthetic construct
MGSGPSNFVNHAPGEAIVTGLEKGADKVAGTISHTIFVEIVSSSTGIIIAVGIFAFIFSFLYKLLQWYNRKSKNKKRKEQIREQIELGLLSYGAGVASLPLLNVIAHNPGSVISATPIYKGPCTGVPNSRLLQITSGTAEENTRILNHDGRNPDGSINV

SEQ ID NO: 204 - protein, 23 aa, synthetic construct
IVSSSTGIIIAVGIFAFIFSFLY

SEQ ID NO: 205 - protein, 38 aa, synthetic construct
MGSGPSNFVNHAPGEAIVTGLEKGADKVAGTISHTIWE

SEQ ID NO: 206 - protein, 49 aa, synthetic construct
KLLQWYNRKSKNKKRKEQIREQIELGLLSYGAGVASLPLLNVIAHNPGS

SEQ ID NO: 207 - protein, 48 aa, synthetic construct
VISATPIYKGPCTGVPNSRLLQITSGTAEENTRILNHDGRNPDGSINV

SEQ ID NO: 208 - protein, 228 aa, synthetic construct
SDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK